(19) United States
(12) Patent Application Publication
(10) Pub. No.: US 2022/0257793 A1
(43) Pub. Date: Aug. 18, 2022

(54) COMPOSITIONS AND USE OF ENGINEERED MYOGENIC CELLS

(71) Applicant: Institute of Zoology, Chinese Academy of Sciences, Beijing (CN)

(72) Inventor: Shyh Chang Ng, Beijing (CN)

(21) Appl. No.: 17/614,303

(22) PCT Filed: May 27, 2020
(86) PCT No.: PCT/CN2020/092615

§ 371 Date: Nov. 24, 2021

(30) Foreign Application Priority Data
May 29, 2019 (CN) PCT/CN2019/088977

(51) Int. Cl. A61K 48/00; A61K 9/00; A61L 27/38; A61L 27/50; A61L 27/52; A01K 67/027; C12N 5/077; C07K 14/62; C12N 9/64

(52) U.S. Cl. CPC A61K 48/005; A61K 48/0075; A61K 9/0019; A61L 27/3826; A61L 27/50; A61K 35/34; A61L 27/3895; A01K 67/0275; C12N 5/0658; C07K 14/62; C12N 9/644; A61L 27/52

(57) ABSTRACT

Provided are compositions and methods of using engineered myogenic cells for delivery of an agent to an individual. Also provided are methods of producing reprogrammed myogenic cells from adult myogenic cells, and use of the reprogrammed myogenic cells for therapy and agent delivery.

### CROSS-REFERENCE TO RELATED APPLICATIONS

This application claims priority benefit of International Patent Application No. PCT/CN2019/088977 filed May 29, 2019, the contents of which are incorporated herein by reference in their entirety.

### FIELD OF THE INVENTION

The present application relates to engineered myogenic cells, including reprogrammed myogenic cells. The present application also relates to use of engineered myogenic cells for therapy and for agent delivery.

### BACKGROUND OF THE INVENTION

Drug delivery systems are integral and enabling components of many therapeutic products. However, conventional drug delivery systems fail to address significant unmet needs in affordable, personalized and long-term delivery of therapeutic agents, especially among socio-economically disadvantaged patient populations. For example, repeated injections or infusions of biologics are costly and inconvenient for patients with chronic diseases or conditions. Lack of access to basic vitamins and essential nutrients has led to high infant mortality in many developing countries. Vaccines, which require cold chain logistics for distribution, remain unavailable for many parts of the world today.

Cell-based systems have recently been employed for drug delivery. Erythrocytes, platelets, immune cells (e.g., monocytes, neutrophils and T cells), bacterial cells, and neural stem cells have been explored as promising drug carriers (Li T. et al., 2018). However, upon administration, cells used in such systems do not stay localized at the site of administration. Also, due to immunogenicity, exogenous cells typically do not persist in the body over a long period of time. Immunoisolated capsules of cells have been designed to deliver recombinant proteins in animal models of genetic diseases (Stockley T L et al., 2000; van Raamsdonk J M et al., 2002). Yet, a safe, versatile and long-lasting cell-based drug delivery system is in urgent need.

The regenerative properties of muscle stem cells decline with ageing as they enter an irreversibly senescent state, thereby failing to proliferate or differentiate, with important implications for transplantation and regenerative medicine (Lau et al., 2019). The regenerative capacity of skeletal muscles depends on muscle stem cells and muscle progenitors, which proliferate in response to tissue damage, and which either fuse and differentiate to regenerate myofibers or self-renew to restore the pool of stem cells (Blau et al., 2015; Buckingham and Relaix, 2015; Cheung and Rando, 2013; Yin et al., 2013). In contrast to aged animals, whose pro-senescent stem cells fail to proliferate and differentiate properly in response to injury, juvenile animals are able to manifest a more robust regenerative response in general. For instance, the larval tissues of many insects and amphibians can regenerate tissues after injuries, but their adult tissues cannot (Shah et al., 2011; Smith-Bolton et al., 2009). Young fish can regenerate their tissues more robustly than older ones (Anchelin et al., 2011), while fetal and perinatal mammals regenerate multiple tissues more robustly than adult mammals (Deuchar, 1976; Porrello et al., 2011; Sadek et al., 2014). This positive correlation between juvenility and tissue regeneration has long been discussed by Charles Darwin and others (Darwin et al., 1887; Pearson, 1984), but the precise mechanisms that underlie juvenility and rejuvenation had remained unclear.

Skeletal muscles constitute ˜40% of the young human body mass. Sarcopenia is the gradual decline of skeletal muscle mass and function with ageing. With ageing, muscles manifest a profound regenerative defect that contributes to elderly frailty in sarcopenia and cachexia. Both changes in the extrinsic microenvironment and stem cell-intrinsic mechanisms may contribute to this regenerative decline (Blau et al., 2015; Grounds, 2014; Lee et al., 2012). Recent studies have demonstrated that both the numbers and the functionality of adult muscle stem cells decline with ageing, especially after geroconversion and senescence (Bernet et al., 2014; Brack and Rando, 2012; Cosgrove et al., 2014; Price et al., 2014; Sousa-Victor et al., 2014; Tierney et al., 2014). In contrast, embryonic, fetal and perinatal muscle progenitors are widely known to possess extended proliferative capacities, compared to adult muscle progenitors, which in turn possess higher proliferative capacities than aged adult muscle progenitors (Blau et al., 1983; Decary et al., 1996, 1997; Tierney et al., 2016). This is despite the fact that embryonic and perinatal progenitors also sustained numerous rounds of mitosis during embryogenesis and fetal development. For instance, it is known that Pax3+ muscle progenitors give rise to all embryonic, fetal, and adult myoblasts and myofibers (Buckingham and Relaix, 2015; Engleka et al., 2005; Hutcheson et al., 2009; Schienda et al., 2006). While it is widely known that muscle progenitors' lifespan inexorably decline with development and ageing, the molecular principles responsible for this phenomenon have remained incompletely understood.

The disclosures of all publications, patents, patent applications and published patent applications referred to herein are hereby incorporated herein by reference in their entirety.

### BRIEF SUMMARY OF THE INVENTION

The present application provides compositions and methods of using engineered myogenic cells (e.g., muscle stem cells, myoblasts, and/or differentiated cells thereof) for agent delivery. Also provided are methods of producing reprogrammed myogenic cells (e.g., rejuvenated and/or de-differentiated myogenic cells) from adult myogenic cells, and use of the reprogrammed myogenic cells in cell-based therapy or agent delivery.

One aspect of the present application provides a method of delivering an agent to an individual in need thereof, comprising locally administering to the individual a composition comprising engineered myogenic cells, wherein the engineered myogenic cells comprise muscle stem cells, myoblasts and/or differentiated cells thereof, wherein the engineered myogenic cells engraft at the site of administration in the individual, and wherein the engineered myogenic cells are genetically modified to allow delivery of the agent to the individual. In some embodiments, the composition is administered intramuscularly. In some embodiments, the composition is administered subcutaneously.

In some embodiments according to any one of the methods described above, the engineered myogenic cells comprise muscle stem cells. In some embodiments, the muscle stem cells are reprogrammed (e.g., rejuvenated and/or de-differentiated) muscle stem cells produced from adult myogenic cells (e.g., myoblasts). In some embodiments, the muscle stem cells are transdifferentiated muscle stem cells produced from adult somatic cells. In some embodiments, the muscle stem cells are Pax7⁺ Pax3⁺ MyoD⁻ myogenin⁻, Pax7⁺ Pax3⁻ MyoD⁻ myogenin⁻, and/or Pax7⁻ Pax3⁺ MyoD⁻ myogenin⁻ cells.

In some embodiments according to any one of the methods described above, the engineered myogenic cells comprise myoblasts. In some embodiments, the myoblasts are produced from embryonic stem cells (ESC) or induced pluripotent stem cells (iPSC). In some embodiments, the myoblasts are reprogrammed (e.g., rejuvenated and/or de-differentiated) myoblasts produced from adult myogenic cells (e.g., myoblasts). In some embodiments, the myoblasts are transdifferentiated myoblasts produced from adult somatic cells. In some embodiments, the myoblasts are Pax7⁻ Pax3⁺ MyoD⁺ myogenin⁻ and/or Pax7⁺ Pax3⁻ MyoD⁺ myogenin⁻ cells.

In some embodiments according to any one of the methods described above, the engineered myogenic cells comprise myocytes produced from muscle stem cells or myoblasts. In some embodiments, the myocytes are differentiated from myoblasts, wherein the myoblasts are produced from ESC or iPSC. In some embodiments, the myocytes are differentiated from reprogrammed (e.g., rejuvenated and/or de-differentiated) muscle stem cells or myoblasts produced from adult myogenic cells. In some embodiments, the myocytes are differentiated from reprogrammed (e.g., transdifferentiated) muscle stem cells or myoblasts produced from adult somatic cells. In some embodiments, the myocytes are Pax3⁻ Pax7⁻ MyoD⁺ myogenin⁺ cells.

In some embodiments according to any one of the methods described above, the engineered myogenic cells comprise reprogrammed myogenic cells. In some embodiments, the engineered myogenic cells comprise reprogrammed myogenic cells produced from adult myogenic cells. In some embodiments, the reprogrammed myogenic cells are rejuvenated myogenic cells produced from adult myogenic cells. In some embodiments, the reprogrammed myogenic cells are de-differentiated myogenic cells produced from adult myogenic cells. In some embodiments, the reprogrammed myogenic cells are transdifferentiated myogenic cells from adult somatic cells. In some embodiments, the reprogrammed myogenic cells comprise muscle stem cells, myoblasts, and/or myocytes. In some embodiments, the engineered myogenic cells comprise myotubes or myofibers differentiated from the reprogrammed myogenic cells.

In some embodiments according to any one of the methods described above, the composition is a suspension of engineered myogenic cells. In some embodiments, the composition is administered by injection.

In some embodiments according to any one of the methods described above, the composition is a muscle construct comprising the engineered myogenic cells. In some embodiments, the composition is administered as a local implant. In some embodiments, the muscle construct comprise myotubes. In some embodiments, the muscle construct comprise myofibers. In some embodiments, the muscle construct comprises PAX7⁺ myogenic cells.

In some embodiments according to any one of the methods described above, the composition further comprises a carrier. In some embodiments, the engineered myogenic cells are intermixed with the carrier. In some embodiments, the carrier comprises extracellular matrix (ECM) molecules. In some embodiments, the carrier comprises MATRIGEL®. In some embodiments, the carrier comprises a cell adhesion molecule. In some embodiments, the cell adhesion molecule is fibrin. In some embodiments, the amount of the carrier in the composition is no more than about 95% (weight/weight).

In some embodiments according to any one of the methods described above, the engineered myogenic cells are not produced from an immortalized cell line. In some embodiments, the engineered myogenic cells are non-tumorigenic.

In some embodiments according to any one of the methods described above, the engrafted engineered myogenic cells form a muscle tissue in the individual, wherein the muscle tissue allows delivery of the agent to the individual. In some embodiments, the muscle tissue produces the agent. In some embodiments, production of the agent is inducible. In some embodiments, production of the agent is constitutive. In some embodiments, the muscle tissue allows local delivery of the agent to the individual. In some embodiments, the muscle tissue allows systemic delivery of the agent to the individual. In some embodiments, the muscle tissue recruits blood vessels from surrounding tissues in the individual. In some embodiments, the muscle tissue is fused to surrounding tissues in the individual. In some embodiments, the muscle tissue is removed from the individual after delivery of the agent to the individual for at least about 2 months (e.g., at least about any one of 3 months, 6 months, 1 year, 5 years, 10 years or more).

In some embodiments according to any one of the methods described above, the composition is administered once to the individual. In some embodiments, the agent is delivered to the individual for at least about 2 months upon administration of the engineered myogenic cells (e.g., at least about any one of 3 months, 6 months, 1 year, 5 years, 10 years or more).

In some embodiments according to any one of the methods described above, the engineered myogenic cells comprise a heterologous nucleic acid sequence encoding the agent. In some embodiments, the agent is selected from the group consisting of a metabolite, a nutrient, a small molecule drug, a biologic, a virus, an extracellular vesicle, a vaccine and a reporter. In some embodiments, the agent is secreted by the engineered myogenic cells. In some embodiments, the agent is expressed on the cell surface of the engineered myogenic cells. In some embodiments, the agent diffuses from the engrafted myogenic cells to the surrounding tissues in the individual. In some embodiments, the agent is actively transported from the engrafted myogenic cells to the surrounding tissues. In some embodiments, the agent is delivered from the engrafted myogenic cells to the surrounding tissues via cell-cell bridges and/or nanotubes. In some embodiments, the agent is delivered via fusion of the engrafted myogenic cells to the surrounding tissue. In some embodiments, the agent is delivered from the engrafted myogenic cells to the surrounding tissues via cell-cell junctions.

In some embodiments according to any one of the methods described above, the engineered myogenic cells allow delivery of a plurality of agents.

In some embodiments according to any one of the methods described above, the composition comprises at least about 10⁵ of the engineered myogenic cells.

In some embodiments according to any one of the methods described above, the engineered myogenic cells are autologous. In some embodiments, the engineered myogenic cells are allogeneic. In some embodiments, the engineered myogenic cells are non-immunogenic to the individual.

In some embodiments according to any one of the methods described above, the individual is a human individual. In some embodiments, the individual is no more than about 3 years old. In some embodiments, the individual is about 18 years old or older.

One aspect of the present application provides a method of treating or diagnosing a disease or condition in an individual, comprising delivering an agent to the individual using the method according to any one of the methods described above, wherein the agent treats or diagnoses the disease or condition. In some embodiments, the disease or condition is a chronic disease or condition. In some embodiments, the disease or condition is diabetes or hemophilia.

One aspect of the present application provides a method of preparing a non-human animal model of a disease or condition, comprising delivering an agent to a non-human animal using the method according to any one of the methods described above, wherein the agent is associated with the disease or condition. In some embodiments, the agent is delivered to the bloodstream of the non-human animal. In some embodiments, the agent is a human protein, RNA or metabolite. In some embodiment, the non-human animal is selected from the group consisting of mammals (e.g., pig, cow, sheep, goat, horse, etc.), birds (e.g., chicken, duck, turkey, etc.), fish, invertebrates, reptiles and amphibians. In some embodiments, the non-human animal is a rodent, such as a mouse or a rat. In some embodiments, the non-human animal is a non-human primate.

One aspect of the present application provides a composition comprising engineered myogenic cells, a hydrogel carrier comprising extracellular matrix molecules, and fibrin, wherein the engineered myogenic cells comprise muscle stem cells, myoblasts and/or differentiated cells thereof, and wherein the engineered myogenic cells are intermixed with the carrier. In some embodiments, the carrier comprises MATRIGEL®. In some embodiments, the amount of the carrier in the composition is no more than about 95% (weight/weight). In some embodiments, the composition is a muscle construct. In some embodiments, the composition is anon-human meat product for consumption.

One aspect of the present application provides a kit for delivery of an agent to an individual, comprising a composition comprising engineered myogenic cells, wherein the engineered myogenic cells comprise muscle stem cells, myoblasts and/or differentiated cells thereof, and wherein the engineered myogenic cells are genetically modified to allow delivery of the agent; and a device for local administration of the composition to the individual.

In some embodiments according to any one of the compositions or kits described above, the engineered myogenic cells comprise muscle stem cells. In some embodiments, the muscle stem cells are reprogrammed (e.g., rejuvenated and/or de-differentiated) muscle stem cells produced from adult myogenic cells (e.g., myoblasts). In some embodiments, the muscle stem cells are transdifferentiated muscle stem cells produced from adult somatic cells. In some embodiments, the muscle stem cells are Pax7⁺ Pax3⁺ MyoD⁻ myogenin⁻, Pax7⁺ Pax3⁻ MyoD⁻ myogenin⁻, and/or Pax7⁻ Pax3⁺ MyoD⁻ myogenin⁻ cells.

In some embodiments according to any one of the compositions or kits described above, the engineered myogenic cells comprise myoblasts. In some embodiments, the myoblasts are produced from embryonic stem cells (ESC) or induced pluripotent stem cells (iPSC). In some embodiments, the myoblasts are reprogrammed (e.g., rejuvenated and/or de-differentiated) myoblasts produced from adult myogenic cells (e.g., myoblasts). In some embodiments, the myoblasts are transdifferentiated myoblasts produced from adult somatic cells. In some embodiments, the myoblasts are Pax7⁻ Pax3⁺ MyoD⁺ myogenin⁻ and/or Pax7⁺ Pax3⁻ MyoD⁺ myogenin⁻ cells.

In some embodiments according to any one of the compositions or kits described above, the engineered myogenic cells comprise myocytes produced from muscle stem cells or myoblasts. In some embodiments, the myocytes are differentiated from myoblasts, wherein the myoblasts are produced from ESC or iPSC. In some embodiments, the myocytes are differentiated from reprogrammed (e.g., rejuvenated and/or de-differentiated) muscle stem cells or myoblasts produced from adult myogenic cells. In some embodiments, the myocytes are differentiated from reprogrammed (e.g., transdifferentiated) muscle stem cells or myoblasts produced from adult somatic cells. In some embodiments, the myocytes are Pax3⁻ Pax7⁻ MyoD⁺ myogenin⁺ cells.

In some embodiments according to any one of the compositions or kits described above, the engineered myogenic cells comprise reprogrammed myogenic cells. In some embodiments, the engineered myogenic cells comprise reprogrammed myogenic cells produced from adult myogenic cells. In some embodiments, the reprogrammed myogenic cells are rejuvenated myogenic cells produced from adult myogenic cells. In some embodiments, the reprogrammed myogenic cells are de-differentiated myogenic cells produced from adult myogenic cells. In some embodiments, the reprogrammed myogenic cells are transdifferentiated myogenic cells from adult somatic cells.

In some embodiments according to any one of the compositions or kits described above, the composition is a suspension of the engineered myogenic cells. In some embodiments, the composition is a muscle construct comprising the engineered myogenic cells. In some embodiments, the composition is administered as a local implant. In some embodiments, the muscle construct comprise myotubes. In some embodiments, the muscle construct comprise myofibers. In some embodiments, the muscle construct comprises PAX7⁺ myogenic cells.

In some embodiments according to any one of the compositions or kits described above, the engineered myogenic cells are not produced from an immortalized cell line. In some embodiments, the engineered myogenic cells are non-tumorigenic.

Also provided are compositions comprising engineered myogenic cells for use in delivering an agent to an individual and use of a composition comprising engineered myogenic cells in the preparation of a medicament for delivery of an agent to an individual in need thereof, wherein the composition is according to any one of the compositions described above.

Another aspect of the present application provides a method of producing a reprogrammed myogenic cell from an adult myogenic cell, comprising: (a) introducing into the adult myogenic cell an embryonic regulator, a telomere-associated regulator, and a cell cycle regulator to provide a transduced myogenic cell; and (b) culturing the transduced myogenic cell under conditions to obtain a reprogrammed myogenic cell. In some embodiments, the reprogrammed myogenic cell is a muscle stem cell. In some embodiments, the reprogrammed myogenic cell is a myoblast. In some embodiments, the reprogrammed myogenic cell is a myocyte.

In some embodiments according to any one of the methods of producing reprogrammed myogenic cells described above, the reprogrammed myogenic cell is rejuvenated. In some embodiments, the adult myogenic cells express high levels of p21^{WAF1}, p16^{INK4a} and let-7 microRNAs. In some embodiments, the reprogrammed myogenic cell expresses low levels of p21^{WAF1}, p16^{INK4a}, and let-7 microRNAs. In some embodiments, the adult myogenic cell can undergo no more than about 30 population doublings. In some embodiments, the reprogrammed myogenic cell can undergo at least about 90 population doublings. In some embodiments, the reprogrammed myogenic cell is de-differentiated. In some embodiments, the adult myogenic cells do not express PAX3 or express a low level of PAX3. In some embodiments, the reprogrammed myogenic cell is PAX3⁺.

In some embodiments according to any one of the methods of producing reprogrammed myogenic cells described above, the embryonic regulator is LIN28A. In some embodiments, the telomere-associated regulator is TERT (e.g., hTERT). In some embodiments, the cell cycle regulator is a p53 inhibitor. In some embodiments, the p53 inhibitor is a short hairpin RNA (shRNA) targeting p53. In some embodiments, step (a) comprises introducing into the adult myogenic cell a first nucleic acid encoding a LIN28A protein, a second nucleic acid encoding a PERT protein, and a third nucleic acid encoding a shRNA targeting p53. In some embodiments, the first nucleic acid, the second nucleic acid and/or the third nucleic acid are present in one or more vectors. In some embodiments, the one or more vectors are viral vectors.

In some embodiments according to any one of the methods of producing reprogrammed myogenic cells described above, the adult myogenic cell is a human cell. In some embodiments, the transduced myogenic cell is cultured in a medium comprising Dulbecco's Modified Eagle Medium (DMEM) with about 20% fetal bovine serum (FBS).

One aspect of the present application provides a reprogrammed myogenic cell prepared by the method according to any one of the methods of producing reprogrammed myogenic cells described above.

Another aspect of the present application provides a method of delivering an agent to an individual in need thereof, comprising locally administering to the individual a composition comprising an effective amount of the reprogrammed myogenic cells according to any one of the reprogrammed myogenic cells described above or differentiated cells thereof, wherein the reprogrammed myogenic cell or differentiated cells thereof are genetically modified to allow delivery of the agent to the individual.

Also provided is a method of treating a muscle disease or condition in an individual in need thereof, comprising administering to the individual an effective amount of reprogrammed myogenic cells according to any one of the reprogrammed myogenic cells described above or differentiated cells thereof. In some embodiments, the adult myogenic cell is obtained from the individual. In some embodiments, the disease or condition is sarcopenia or cachexia.

Further provided are compositions comprising reprogrammed myogenic cells for use in agent delivery or treatment and use of a composition comprising reprogrammed myogenic cells in the preparation of a medicament for delivery of an agent to an individual or treatment of a disease in an individual.

Yet another aspect of the present application provides a kit for producing reprogrammed myogenic cells, comprising: (a) a first nucleic acid encoding a LIN28A protein, (b) a second nucleic acid encoding a TERT protein, and (c) a p53 inhibitor (e.g., a third nucleic acid encoding a shRNA targeting p53).

Further provided are pharmaceutical compositions, kits, and articles of manufacture for use in any one of the methods described above.

These and other aspects and advantages of the present invention will become apparent from the subsequent detailed description and the appended claims. It is to be understood that one, some, or all of the properties of the various embodiments described herein may be combined to form other embodiments of the present invention.

### BRIEF DESCRIPTION OF THE DRAWINGS

FIG. 1 shows intramuscular engraftment of engineered myogenic (“iMusc”) and human skeletal muscle (“hSkM”) cells one year after orthotopic transplantation into the tibialis anterior muscles of a NOD-Scid gamma mouse.

FIG. 2 shows hematoxylin and eosin (H&E) histology staining of cross-sections of the intramuscular (tibialis anterior) injection sites (black arrows) in the mouse of FIG. 1. Left panel shows the injection site of iMusc cells, and right panel shows the injection site of hSkM cells.

FIG. 3 shows immunofluorescence staining for the skeletal muscle marker pan-myosin heavy chain and the human mitochondrial antigen in cross-sections of the intramuscular iMusc grafts, 1 year after transplantation.

FIG. 4 shows immunofluorescence staining for the muscle intermediate filament marker desmin and the skeletal muscle sarcomeric Z-disc striation marker α-actinin, in cross-sections of the intramuscular iMusc grafts, 1 year after transplantation.

FIG. 5 shows immunofluorescence staining for the skeletal muscle marker pan-myosin heavy chain (MHC), the adult fast-twitch muscle MHC isoform, and the myotube specification factor myogenin, in cross-sections of the intramuscular iMusc grafts, 1 year after transplantation.

FIG. 6 shows culture of human iMusc construct in vitro, by seeding iMusc cells into a MATRIGEL®-fibrin scaffold, anchored down by steel pins and silk sutures, and allowed to grow and mature for 6 weeks. Electric wires were connected to the steel pins to stimulate muscle contractions of the iMusc construct.

FIG. 7 shows immunofluorescence staining for the muscle stem cell marker PAX7, the myotube marker myogenin, and the skeletal muscle marker pan-myosin heavy chain (MHC), in cross-sections of iMusc constructs, demonstrating the co-culture of PAX7⁺ iMusc cells and myogenin/pan-MHC⁺ iMusc-myotubes in the MATRIGEL®-fibrin scaffold.

FIG. 8 shows a photomicrograph of a representative iMusc construct, anastomosed with mouse blood vessels, 8 weeks after subcutaneous transplantation under the dorsal skin of a NOD-Scid gamma mouse.

FIG. 9 shows immunofluorescence staining for PAX7 and pan-myosin heavy chain (MHC) in cross-sections of the subcutaneous iMusc construct, depicting the persistence of both PAX7⁺ iMusc cells and pan-MHC⁺ iMusc-myotubes in the subcutaneous environment 1 year after implantation.

FIG. 10 shows immunofluorescence staining for the adult fast-twitch muscle MHC isoform, and pan-myosin heavy chain (MHC), in cross-sections of the subcutaneous space harboring an iMusc construct, depicting the persistence of adult fast MHC⁺ iMusc-myotubes in the subcutaneous environment 1 year after implantation.

FIG. 11 shows hematoxylin and eosin (H&E) histology staining of cross-sections of the subcutaneous iMusc construct. The iMusc cells, anastomosed capillaries, and mouse skin can be observed 1 year after implantation.

FIG. 12 shows gene expression of myogenesis markers, as determined by qRT-PCR, for the iMusc myofiber (MF) construct in vivo and in vitro, relative to primary human skeletal muscle (hSkM) myoblasts (MB), myocytes (MC), or myotubes (MT).

FIG. 13 shows a photomicrograph of the result of implanting iMusc-derived myoblasts, encapsulated in a hemispheroid of MATRIGEL®-fibrin matrix (inset), 1 year after implantation into the quadriceps muscles of a NOD-Scid gamma mouse.

FIG. 14 shows H&E histology of the iMusc hemispheroid implant into the quadriceps muscles (dotted outline), 1 year after implantation into NOD-Scid gamma mice.

FIG. 15 shows immunofluorescence staining for the sarcomeric Z-disc striation marker α-actinin in cross-sections of the iMusc hemispheroid graft, 1 year after implantation.

FIG. 16 shows immunofluorescence staining for CD31⁺ (red) vascular capillary recruitment into the iMusc (purple) hemispheroid graft.

FIG. 17 shows immunofluorescence staining for the GFP transgene (purple), skeletal muscle marker pan-myosin heavy chain (MHC; green) and the human lamin A/C antigen (red) in cross-sections of the GFP-iMusc-derived myocyte graft, 1 year after transplantation into uninjured tibialis anterior muscles of NOD-Scid gamma mice.

FIG. 18 shows immunofluorescence staining for the GFP transgene (purple), skeletal muscle marker pan-myosin heavy chain (MHC; green) and the human lamin A/C antigen (red) in cross-sections of the GFP-iMusc-derived myoblast graft, 1 year after transplantation into uninjured tibialis anterior muscles of NOD-Scid gamma mice.

FIG. 19 shows immunofluorescence staining for the GFP transgene (purple), skeletal muscle marker pan-myosin heavy chain (MHC; green) and the human lamin A/C antigen (red) in transverse sections of the GFP-iMusc-derived myoblast graft, 1 year after transplantation into cardiotoxin-injured tibialis anterior muscles of NOD-Scid gamma mice.

FIG. 20 shows immunofluorescence staining for the GFP transgene (purple), skeletal muscle marker pan-myosin heavy chain (MHC; green) and the human lamin A/C antigen (red) in longitudinal sections of the GFP-iMusc-derived myoblast graft, 1 year after transplantation into cardiotoxin-injured tibialis anterior muscles of NOD-Scid gamma mice.

FIG. 21 shows immunofluorescence staining for the GFP+ (purple); human lamin A/C+ (red) iMusc cells that fused with mouse myofibers in vivo, 1 year after transplantation into cryoinjured tibialis anterior muscles of NOD-Scid gamma mice.

FIG. 22A-G show results of a mini-screen for factors that extend the self-renewal of old adult primary human skeletal muscle (HSKM) progenitors. FIG. 22A shows quantitative RT-PCR for mRNAs of cell cycle inhibitors in old adult HSKM myoblasts, relative to young adult HSKM myoblasts. FIG. 22B shows quantitative RT-PCR for let-7 microRNAs in old adult HSKM myoblasts, relative to young adult HSKM myoblasts. FIG. 22C shows quantitative RT-PCR for telomere length in old adult HSKM myoblasts, relative to young adult HSKM myoblasts. FIG. 22D shows population doubling curves for young HSKM myoblasts (black), and young adult HSKM myoblasts transduced with viral sh-p53 (S, pink), and LIN28A (LS, green), or hTERT (TS, purple), or LIN28A and hTERT (LTS, red). While young adult HSKM and other transgenic myoblasts started to undergo senescence by 60 days, before the 30^{th} population doubling, the LTS myoblasts (red) continued to proliferate steadily beyond 120 days and beyond the 90^{th} population doubling. FIG. 22E shows quantification of senescence-associated β-galactosidase positive (SA-β-gal+) cells in 100-day-old adult HSKM myoblasts, relative to 100-day-old LTS myoblasts. FIG. 22F shows quantitative RT-PCR for p27^{KIP1}, p21^{WAF1}, p16^{INK4a}, and let-7 microRNAs in LTS myoblasts, relative to young adult HSKM myoblasts. FIG. 22G shows immunofluorescence staining for the myotube protein marker myosin heavy chain (MHC) in young HSKM and LTS myoblasts, relative to old HSKM myoblasts and immortalized (hTERT-CyclinD1-CDK4^{K24C}) myoblasts, which were subjected to myogenic differentiation in myotube media. Cells were counterstained with DAPI to visualize the myonuclei. Scale bars 100 μm. * P<0.05, ** P<0.01, *** P<0.001.

FIG. 23 shows expression of p53 protein during muscle progenitor ageing and differentiation. Western blot for p53 protein, relative to tubulin protein, in young and old adult HSKM and LTS myoblasts and myotubes.

FIG. 24 shows population doubling curves for young HSKM and other transgenic myoblasts. Young HSKM myoblasts (black), and young adult HSKM myoblasts transduced with hTERT and shp53 (TS, orange), or LIN28A hTERT and shp53 (LTS, red) showed an increasing order of proliferation rate. Lentiviral shRNA knockdowns of p16^{INK4a} (sh-p16, purple), p14^{Arf} (sh-p14, blue), and RB1 (sh-RB1, green) were also attempted, but were not followed up upon as they appeared to be prematurely senescent.

FIG. 25 shows expression levels of the LTS factors. Quantitative RT-PCR for mRNAs of Lin28, hTERT, and p53 in LTS myoblasts, relative to young adult HSKM myoblasts. ** P<0.01, *** P<0.001

FIG. 26 shows effects of the LTS factors on telomere length. Quantitative RT-PCR for telomere length in young LTS myoblasts, relative to young adult HSKM myoblasts. ** P<0.01.

FIG. 27 shows effects of the LTS factors on muscle progenitor differentiation. Immunofluorescence staining for the myotube protein markers α-actinin (green) and nuclear myogenin (red) in young HSKM myotubes, relative to LTS myotubes. Cells were counterstained with DAPI (blue) to visualize the myonuclei. Scale bars 100 μm.

FIG. 28 shows effects of the LTS factors on myogenic differentiation markers. Quantitative RT-PCR for mRNAs of myogenic markers in LTS myotubes, relative to young adult HSKM myotubes.

FIGS. 29A-29K show that LTS rejuvenates aged muscle progenitors' regenerative properties in vivo. FIG. 29A shows aschematic for derivation and treatment of aged patients' myoblasts for orthotopic transplantation into NOD SCID gamma (NSG) mice. FIG. 29B shows population doubling curves for aged cachexia patient #1's HSKM myoblasts (cHSKM-1, light green), aged cachexia patient #14's HSKM myoblasts (cHSKM-14, light blue), and their corresponding LTS-treated lines (cLTS-1 and cLTS-14, green and blue). FIG. 29C shows quantitative RT-PCR for mRNAs of Lin28, Tert, p53 and p21 in cLTS myoblasts (green and blue), relative to cachectic patients' cHSKM myoblasts (light green and light blue). FIG. 29D shows immunofluorescence staining for GFP⁺ cLTS1 myoblasts and myotubes after orthotopic transplantation into NSG mice' muscles (20× magnification). FIG. 29E shows immunofluorescence staining for GFP⁺ cLTS1 myofibers after orthotopic transplantation into NSG mice' muscles (40× magnification). FIG. 29F shows immunofluorescence staining for GFP⁺ myofiber domains after orthotopic transplantation of GFP⁺ cLTS1 myoblasts into NSG mice' muscles (40× magnification). FIG. 29G shows immunofluorescence staining for GFP in transverse sections of NSG mice' muscles, after orthotopic transplantation of GFP⁺ cLTS1 myoblasts (40× magnification). FIG. 29H shows quantification of engraftment efficiency after transplantation of GFP⁺ cLTS1 myoblasts. FIG. 29I shows representative NSG mouse carcass with orthotopic graft of LTS myoblasts into NSG mice' muscles. FIGS. 29J-29K shows representative karyotype analysis of old TS and LTS myoblasts, which are [41,X,−X,dic(8;17)(q24.3;p13),−16,−18,−20,+mar] (FIG. 29J), and [46,XX] (FIG. 29K) respectively. * P<0.05, ** P<0.01, *** P<0.001.

FIGS. 30A-30C show that LTS muscle progenitors are less aged and less differentiated. FIG. 30A shows a plot of Principal Components Analysis (PCA) of transcriptomic data for LTS myoblasts and myotubes, relative to HSKM young, intermediate and old myoblasts, and young HSKM myotubes. FIG. 30B shows a Western blot for PAX3 and TWIST2 protein, relative to GAPDH protein, in young HSKM, TS and LTS myoblasts and myotubes. FIG. 30C shows quantitative RT-PCR for PAX3, MYOD, MYF5, MYOG, ACTA1, MYH1, MYH3, MYH8 and MYH7 in LTS myoblasts, relative to young adult HSKM myoblasts.

FIGS. 31A-31E show that Let-7 repression is insufficient to explain LTS rejuvenation of muscle progenitors. FIG. 31A show quantitative RT-PCR for let-7 microRNAs in TS and LTS myoblasts, relative to young adult HSKM myoblasts, after control (Ctrl), let-7a, let-7b, or combined let-7a and let-7b (let-7a+b) mimic transfection. FIG. 31B show cell counts after control RNAi (Ctrl), let-7a, let-7b, or combined let-7a and let-7b (let-7a+b) mimic transfection in TS and LTS myoblasts, relative to young adult HSKM myoblasts. FIG. 31C show quantification of SA-β-gal+ cells in TS and LTS myoblasts, relative to young adult HSKM myoblasts, after control RNAi (Ctrl RNAi), let-7a, let-7b, or combined let-7a and let-7b (let-7a+b) mimic transfection. FIG. 31D show a Western blot for IMP1/2/3 and HMGA2 protein, relative to GAPDH protein, in young adult HSKM, TS and LTS myoblasts, after control (Ctr), let-7a (a), let-7b (b), or combined let-7a and let-7b (a & b) mimic transfection. FIG. 31E shows gene Set Enrichment Analysis (GSEA) of transcriptomic profiles of LTS myoblasts.

FIGS. 31A-31L show metabolic effects of LIN28A in LTS muscle progenitors. FIG. 31A shows Seahorse analysis of basal OxPhos in LTS myoblasts, relative to young adult HSKM myoblasts. FIG. 31B shows Seahorse analysis of maximal OxPhos in LTS myoblasts, relative to young adult HSKM myoblasts. FIG. 31C shows Seahorse analysis of glycolysis in LTS myoblasts, relative to young adult HSKM myoblasts. FIG. 31D shows quantification of the mitochondrial membrane potential (Δψₘ) in LTS myoblasts, relative to young adult HSKM myoblasts, according to the JC-1 dye red:green fluorescence ratio. FIG. 31E shows quantification of mitochondrial ROS in LTS myoblasts, relative to young adult HSKM myoblasts, according to the mean percentage oxidation of mito-Grx1-roGFP2 probe. FIG. 31F shows LC-MS/MS quantification of glycolysis metabolites in LTS myoblasts (red) and adult HSKM myoblasts (black). G6P, glucose-6-phosphate. F6P, fructose-6-phosphate. G3P, glyceraldehyde-3-phosphate. FIG. 31G shows LC-MS/MS quantification of Krebs Cycle metabolites in LTS myoblasts (red) and adult HSKM myoblasts (black). aKG, α-ketoglutarate. FIG. 31H shows LC-MS/MS quantification of redox and bioenergetics-related metabolites in LTS myoblasts (red) and adult HSKM myoblasts (black). NADH, reduced NAD. NAD, nicotinamide adenine dinucleotide. GSH, glutathione. GSSG, glutathione disulfide. AMP, adenine monophosphate. ADP, adenine diphosphate. FIG. 31I shows quantification of mitochondrial ROS in young adult HSKM, TS and LTS myoblasts, after treatment with the ROS-modulating drugs paraquat (PQ), hydrogen peroxide (H₂O₂), dithiothreitol (DTT), or vehicle (Veh), according to the mean percentage oxidation of mito-Grx1-roGFP2 probe. FIG. 31J shows quantitative RT-PCR for hypoxia/HIF1A target mRNAs in LTS myoblasts, relative to TS myoblasts, after treatment with vehicle control (CTRL), hydrogen peroxide (H₂O₂), or dithiothreitol (DTT). FIG. 31K shows quantitative RT-PCR for glycolysis-related mRNAs in LTS myoblasts, relative to TS myoblasts, after treatment with vehicle control (CTRL), hydrogen peroxide (H₂O₂), or dithiothreitol (DTT). FIG. 31L shows quantitative RT-PCR for OxPhos-related mRNAs in LTS myoblasts, relative to TS myoblasts, after treatment with vehicle control (CTRL), hydrogen peroxide (H₂O₂), or dithiothreitol (DTT).

FIG. 32 shows effects of the LTS factors on mitochondrial DNA (mtDNA) abundance. Quantitative RT-PCR for mitochondrial NADH-ubiquinone oxidoreductase chain 1 (MT-ND1) and 4 (MT-ND4), relative to the nuclear reference gene B2M, to assess mtDNA copy number in adult HSKM myoblasts, relative to LTS myoblasts. * P<0.05.

FIG. 33 shows effects of the LTS factors on mitochondrial biogenesis. Western blot for mitochondrial citrate synthase protein, relative to GAPDH protein, in TS and LTS myoblasts, relative to adult HSKM myoblasts.

FIGS. 34A-34E show that LTS progenitors are dependent on mtROS-driven HIF1A for proliferation. FIG. 34A shows quantification of the hypoxia-response element (HRE)-luciferase reporter in young adult HSKM, TS and LTS myoblasts, after treatment with dithiothreitol (DTT) or vehicle control. FIG. 34B shows cell counts after treatment with vehicle control (Veh), hydrogen peroxide (H₂O₂), or dithiothreitol (DTT) titrations in TS and LTS myoblasts, relative to young adult HSKM myoblasts. FIG. 34C shows cell counts after treatment with vehicle control (Veh) or the HIF-1a inhibitor KC7F2 in TS and LTS myoblasts, relative to young adult HSKM myoblasts. FIG. 34D shows cell counts after treatment with vehicle control or the glycolysis inhibitor 2-deoxyglycose (2DG) in TS and LTS myoblasts, relative to young adult HSKM myoblasts. FIG. 34E shows a model of how LIN28A prevents muscle progenitor senescence and promotes self-renewal. * P<0.05, ** P<0.01, *** P<0.001.

FIGS. 35A-35B show effects of the LTS factors on 8-oxo-guanine. FIG. 35A shows immunofluorescence staining for the oxidative damage biomarker 8-oxo-guanine (green) in adult HSKM and LTS myoblasts. Cells were counterstained with DAPI (blue) to visualize the myonuclei. FIG. 35B shows quantification of the immunofluorescence signal from 8-oxo-guanine in adult HSKM and LTS myoblasts. * P<0.05.

FIG. 36 shows insulin mRNA levels in differentiated iMusc-Insm cells (i.e., iMusc cells transduced with human proinsulin cDNA) and iMusc-neo cells (i.e., control iMusc cells without transgene).

FIG. 37 shows levels of insulin protein secreted by differentiated iMusc-Insm cells and iMusc-neo cells.

FIG. 38 shows ¹³C-incorporation into fructose-6-phosphate (F6P) by hSkM cells exposed to 48-hour-conditioned media from iMusc-Insm cells and iMusc-neo cells.

FIG. 39 shows plasma insulin levels in diabetic nude mice transplanted with iMusc-Insm cells or iMusc-neo cells.

FIG. 40 shows blood glucose levels in diabetic nude mice transplanted with iMusc-Insm cells or iMusc-neo cells.

FIG. 41 shows human Factor IX (hFIX) mRNA levels in differentiated iMusc-FIX cells (i.e., iMusc cells transduced with hFIX cDNA) and iMusc-neo cells (i.e., control iMusc cells without transgene).

FIG. 42 shows myogenin (Myog) mRNA levels in differentiated iMusc-FIX cells and iMusc-neo cells.

FIG. 43 shows levels of hFIX protein secreted by differentiated iMusc-FIX cells and iMusc-neo cells.

FIG. 44 shows plasma hFIX protein levels in hemophilic nude mice transplanted with iMusc-FIX cells and iMusc-neo cells.

FIG. 45 shows activated partial thromboplastin time (aPTT) of hemophilic nude mice transplanted with iMusc-FIX cells and iMusc-neo cells.

### DETAILED DESCRIPTION OF THE INVENTION

The present application provides compositions and methods of using engineered myogenic cells as delivery vehicle in vivo. In some embodiments, the engineered myogenic cells are myoblasts produced from embryonic stem cells or induced pluripotent stem cells, reprogrammed (e.g., rejuvenated and/or de-differentiated, or transdifferentiated) myogenic cells, or differentiated cells thereof (e.g., myocytes, myotubes and/or myofibers). The compositions are administered locally to the individual to allow engraftment of the engineered myogenic cells in the individual and long-term delivery of exogenous agents to the individual. The engrafted myogenic cells can give rise to a muscle tissue that persists at the site of administration over an extended period of time (e.g., at least about 2 months, 3 months, 6 months, 1 year, 2 years, 5 years, 10 years or more), and has low risk of immunogenicity or tumorigenicity. Surprisingly, the engrafted muscle tissue can recruit vasculature from the surrounding tissues of the individual, which enables systemic delivery of secreted agents via the individual's circulation system. The methods and compositions described herein provide a versatile and cost-effective delivery system for a variety of agents, including nutrients and biologic drugs.

Additionally, the present application provides methods of producing reprogrammed myogenic cells (e.g., rejuvenated myoblasts) from adult myogenic cells by introducing a combination of three factors, such as LIN28A, TERT and a p53 inhibitor, into the adult myogenic cells. The reprogrammed myogenic cells have extended proliferative capacity, maintain a normal karyotype, undergo myogenesis normally in late-passages, and do not manifest tumorigenesis or aberrant lineage differentiation. The reprogrammed myogenic cells also demonstrate significantly enhanced engraftment efficiency compared to primary adult myogenic cells, and thus can be used in the methods of agent delivery described herein, and in other cell-based regenerative therapies.

Accordingly, one aspect of the present application provides a method of delivering an agent to an individual in need thereof, comprising locally (e.g., intramuscularly or subcutaneously) administering to the individual a composition comprising engineered myogenic cells, wherein the engineered myogenic cells comprise muscle stem cells, myoblasts and/or differentiated cells thereof, wherein the engineered myogenic cells engraft at the site of administration in the individual, and wherein the engineered myogenic cells are genetically modified to allow delivery (e.g., local or systemic delivery) of the agent to the individual. In some embodiments, the engineered myogenic cells comprise a heterologous nucleic acid sequence encoding the agent. In some embodiments, the engineered myogenic cells comprise myoblasts produced from ESC or iPSC, or differentiated cells thereof such as myocytes, myotubes and/or myofibers. In some embodiments, the engineered myogenic cells comprise reprogrammed myoblasts, or differentiated cells thereof such as myocytes, myotubes and/or myofibers. In some embodiments, the composition is a muscle construct comprising the engineered myogenic cells or differentiated cells thereof and a hydrogel carrier (e.g., MATRIGEL® and fibrin).

Another aspect of the present application provides a method of producing a reprogrammed (e.g., rejuvenated and/or de-differentiated) myogenic cell from an adult myogenic cell, comprising: (a) introducing into the adult myogenic cell an embryonic regulator (e.g., LIN28A), a telomere-associated regulator (e.g., TERT), and a cell cycle regulator (e.g., p53 inhibitor such as a shRNA targeting p53) to provide a transduced myogenic cell; and (b) culturing the transduced myogenic cell under conditions to obtain a reprogrammed myogenic cell.

Methods of treatment, compositions, kits, and articles of manufacture are also provided.

### I. Definitions

Terms are used herein as generally used in the art, unless otherwise defined as follows.

As used herein, “myogenic cells” refer to cells that can proliferate and/or differentiate to give rise to a muscle tissue. Myogenic cells include, but are not limited to, muscle stem cells, myoblasts, myocytes, myotubes, and myofibers. The myogenic cells contemplated herein may give rise to skeletal muscle, smooth muscle, and/or cardiac muscle.

As used herein, a “stem cell” is an undifferentiated cell characterized by the ability of self-renewal through mitotic cell division and the potential to differentiate into progeny cells, including self-renewing progenitors, non-renewing progenitors, and terminally differentiated cells. Among mammalian stem cells, embryonic and somatic stem cells can be distinguished. “Embryonic stem cells” or “ESC” reside in the blastocyst and give rise to embryonic tissues, whereas somatic stem cells reside in adult tissues for the purpose of tissue regeneration and repair. “Muscle stem cells” refer to stem cells found in adult muscle tissues, including for example, satellite cells. Stem cells can be naturally occurring stem cells, or “induced” by reprogramming differentiated cells (e.g., somatic cells) into stem cells using nuclear reprogramming factors. “Induced pluripotent stem cells” or “iPSC” refer to pluripotent stem cells that are prepared artificially from a non-pluripotent cell. The term “pluripotent” or “pluripotency” refers to cells with the ability to give rise to progeny that can undergo differentiation, under appropriate conditions, into cell types that collectively exhibit characteristics associated with cell lineages from the three germ layers (endoderm, mesoderm, and ectoderm). Pluripotent stem cells can contribute to tissues of a prenatal, postnatal or adult organism. A standard art-accepted test, such as the ability to form a teratoma in 8-12 week old SCID mice, can be used to establish the pluripotency of a cell population. However, identification of various pluripotent stem cell characteristics can also be used to identify pluripotent cells. iPSC and methods for preparing iPSC have been described in the art. See, for example, U.S. Pat. No. 8,058,065B2, U.S. Pat. No. 8,129,187B2, U.S. Pat. No. 8,278,104B2 and U.S. Pat. No. 9,499,797B2, which are incorporated herein by reference in their entirety.

As used herein, “muscle progenitor cells” refer to undifferentiated cells that have the potential to differentiate into muscle cells. Muscle progenitor cells include, but are not limited to, muscle stem cells and myoblasts. Primary adult muscle progenitor cells have limited proliferative capacities, upon which they enter a senescent state and lose both proliferative and differentiation capacities. In contrast, embryonic and fetal muscle progenitor cells have heightened proliferative capacities despite many rounds of mitosis, and manifest robust regenerative response upon injury and transplantation.

As used herein, “reprogrammed myogenic cells” refer to artificially manipulated adult myogenic cells that have enhanced proliferative and differentiation capacities, and delayed senescence. Reprogrammed myogenic cells can be developmentally reprogrammed and/or age-reprogrammed. “Developmentally reprogrammed myogenic cells” refer to artificially manipulated adult myogenic cells that are at a less committed developmental stage than the adult myogenic cells from which the reprogrammed myogenic cells are derived from (a process known known as “de-differentiation”), or artificially manipulated adult cells that are in a different developmental lineage than the adult somatic cells from which the reprogrammed myogenic cells are derived from (a process known as “transdifferentiation”). “Age-reprogrammed myogenic cells” and “rejuvenated myogenic cells” are used interchangeably to refer to artificially manipulated adult myogenic cells that can undergo more cycles of proliferation than the adult myogenic cells from which the reprogrammed myogenic cells are derived from.

As used herein, “myoblasts” refer to mononuclear muscle progenitor cells that can differentiate to give rise to muscle cells.

As used herein, “myocytes” refer to mononuclear muscle cells that result from differentiation of muscle progenitor cells.

As used herein, “myotubes” refer to multi-nucleated muscle cells that result from the fusion of myocytes.

As used herein, “myofibers” refer to terminally differentiated, multi-nucleated, and striated muscle cells that develop from myotubes.

As used herein, “engraftment” or “engraft” refers to survival of transplanted cells, construct, or tissue in a host for at least about 7 weeks (such as at least about any one of 2 months, 3 months, 6 months, 7 months, 8 months, 9 months, 10 months, 11 months, 1 year, 2 years, 3 years, 4 years, 5 years, 6 years, 7 years, 8 years, 9 years, 10 years or more). Engrafted cells carry out normal cellular functions, including, for example, proliferation, differentiation, metabolism, gene expression, etc., in the host. The engrafted cells, construct, or tissue may or may not fuse with the surrounding tissue in the host.

As used herein, “treatment” or “treating” is an approach for obtaining beneficial or desired results including clinical results. For purposes of this invention, beneficial or desired clinical results include, but are not limited to, one or more of the following: decreasing one or more symptoms resulting from the disease, diminishing the extent of the disease, stabilizing the disease or condition (e.g., preventing or delaying the worsening of the disease or condition), preventing or delaying the spread of the disease or condition, preventing or delaying the occurrence or recurrence of the disease or condition, delaying or slowing the progression of the disease or condition, ameliorating the disease state, providing a remission (whether partial or total) of the disease or condition, decreasing the dose of one or more other medications required to treat the disease or condition, delaying the progression of the disease or condition, increasing the quality of life, and/or prolonging survival. Also encompassed by “treatment” is a reduction of pathological consequence of the disease or condition. The methods of the invention contemplate any one or more of these aspects of treatment.

The terms “individual,” “subject” and “patient” are used interchangeably herein to describe a mammal, including humans. An individual includes, but is not limited to, human, bovine, ovine, porcine, equine, feline, canine, rodent, or primate. In some embodiments, the individual is human. In some embodiments, an individual suffers from a disease or condition. In some embodiments, the individual is in need of treatment.

As is understood in the art, an “effective amount” refers to an amount of a composition (e.g., engineered myogenic cells) sufficient to produce a desired therapeutic outcome. For therapeutic use, beneficial or desired results include, e.g., decreasing one or more symptoms resulting from the disease or condition (biochemical, histologic and/or behavioral), including its complications and intermediate pathological phenotypes presented during development of the disease or condition, increasing the quality of life of those suffering from the disease or condition, decreasing the dose of other medications required to treat the disease or condition, enhancing effect of another medication, delaying the progression of the disease or condition, and/or prolonging survival of patients.

As used herein, the terms “cell” and “cell culture” are used interchangeably and all such designations include progeny. It is understood that all progeny may not be precisely identical in DNA content, due to deliberate or inadvertent mutations. Variant progeny that have the same function or biological activity as the original cells are included.

It is understood that aspect and embodiments of the invention described herein include “consisting” and/or “consisting essentially of” aspects and embodiments.

Reference to “about” a value or parameter herein includes (and describes) variations that are directed to that value or parameter per se. For example, description referring to “about X” includes description of “X”.

The term “about X-Y” used herein has the same meaning as “about X to about Y.”

As used herein, reference to “not” a value or parameter generally means and describes “other than” a value or parameter. For example, the method is not used to treat cancer of type X means the method is used to treat cancer of types other than X.

As used herein and in the appended claims, the singular forms “a,” “an,” and “the” include plural referents unless the context clearly dictates otherwise.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. Methods of Delivery

The present application provides methods of using engineered myogenic cells or constructs thereof for delivering an agent to an individual in need.

In some embodiments, there is provided a method of delivering an agent to an individual in need thereof, comprising locally administering to the individual a composition comprising engineered myogenic cells, wherein the engineered myogenic cells comprise muscle stem cells, myoblasts and/or differentiated cells thereof, wherein the engineered myogenic cells engraft at the site of administration in the individual, and wherein the engineered myogenic cells are genetically modified to allow delivery of the agent to the individual. In some embodiments, the engineered myogenic cells comprise a heterologous nucleic acid sequence encoding the agent. In some embodiments, the agent is delivered locally to the individual. In some embodiments, the agent is delivered systemically to the individual. In some embodiments, the engineered myogenic cells are not produced from an immortalized cell line. In some embodiments, the engineered myogenic cells comprise muscle stem cells, e.g., Pax7⁺ Pax3⁺ MyoD⁻ myogenin⁻, Pax7⁺ Pax3⁻ MyoD⁻ myogenin⁻, and/or Pax7⁻ Pax3⁺ MyoD⁻ myogenin⁻ cells. In some embodiments, the engineered myogenic cells comprise myoblasts, e.g., Pax7⁻ Pax3⁺ MyoD⁺ myogenin⁻ and/or Pax7⁺ Pax3⁻ MyoD⁺ myogenin⁻ cells. In some embodiments, the engineered myogenic cells comprise cells produced by differentiating muscle stem cells or myoblasts, such as myocytes, myotubes, and/or myofibers. In some embodiments, the engrafted engineered myogenic cells form a muscle tissue in the individual, wherein the muscle tissue allows delivery of the agent to the individual. In some embodiments, the muscle tissue is removed from the individual after delivery of the agent to the individual for at least about 2 months (e.g., at least about any one of 3 months, 6 months, 1 year, 5 years, 10 years or more). In some embodiments, the agent is secreted by the engineered myogenic cells. In some embodiments, the engineered myogenic cells allow delivery of a plurality of agents.

In some embodiments, there is provided a method of delivering an agent to an individual in need thereof, comprising intramuscularly administering to the individual a composition comprising engineered myogenic cells, wherein the engineered myogenic cells comprise muscle stem cells, myoblasts and/or differentiated cells thereof, wherein the engineered myogenic cells engraft at the site of administration in the individual, and wherein the engineered myogenic cells are genetically modified to allow delivery of the agent to the individual. In some embodiments, the engineered myogenic cells comprise a heterologous nucleic acid sequence encoding the agent. In some embodiments, the agent is delivered locally to the individual. In some embodiments, the agent is delivered systemically to the individual. In some embodiments, the engineered myogenic cells are not produced from an immortalized cell line. In some embodiments, the engineered myogenic cells comprise muscle stem cells, e.g., Pax7⁺ Pax3⁺ MyoD⁻ myogenin⁻, Pax7⁺ Pax3⁻ MyoD⁻ myogenin⁻, and/or Pax7⁻ Pax3⁺ MyoD⁻ myogenin⁻ cells. In some embodiments, the engineered myogenic cells comprise myoblasts, e.g., Pax7⁻ Pax3⁺ MyoD⁺ myogenin⁻ and/or Pax7⁺ Pax3⁻ MyoD⁺ myogenin⁻ cells. In some embodiments, the engineered myogenic cells comprise cells produced by differentiating muscle stem cells or myoblasts, such as myocytes, myotubes, and/or myofibers. In some embodiments, the engrafted engineered myogenic cells form a muscle tissue in the individual, wherein the muscle tissue allows delivery of the agent to the individual. In some embodiments, the muscle tissue is removed from the individual after delivery of the agent to the individual for at least about 2 months (e.g., at least about any one of 3 months, 6 months, 1 year, 5 years, 10 years or more). In some embodiments, the agent is secreted by the engineered myogenic cells. In some embodiments, the engineered myogenic cells allow delivery of a plurality of agents.

In some embodiments, there is provided a method of delivering an agent to an individual in need thereof, comprising subcutaneously administering to the individual a composition comprising engineered myogenic cells, wherein the engineered myogenic cells comprise muscle stem cells, myoblasts and/or differentiated cells thereof, wherein the engineered myogenic cells engraft at the site of administration in the individual, and wherein the engineered myogenic cells are genetically modified to allow delivery of the agent to the individual. In some embodiments, the engineered myogenic cells comprise a heterologous nucleic acid sequence encoding the agent. In some embodiments, the agent is delivered locally to the individual. In some embodiments, the agent is delivered systemically to the individual. In some embodiments, the engineered myogenic cells are not produced from an immortalized cell line. In some embodiments, the engineered myogenic cells comprise muscle stem cells, e.g., Pax7⁺ Pax3⁺ MyoD⁻ myogenin⁻, Pax7⁺ Pax3⁻ MyoD⁻ myogenin⁻, and/or Pax7⁻ Pax3⁺ MyoD⁻ myogenin⁻ cells. In some embodiments, the engineered myogenic cells comprise myoblasts, e.g., Pax7⁻ Pax3⁺ MyoD⁺ myogenin⁻ and/or Pax7⁺ Pax3⁻ MyoD⁺ myogenin⁻ cells. In some embodiments, the engineered myogenic cells comprise cells produced by differentiating muscle stem cells or myoblasts, such as myocytes, myotubes, and/or myofibers. In some embodiments, the engrafted engineered myogenic cells form a muscle tissue in the individual, wherein the muscle tissue allows delivery of the agent to the individual. In some embodiments, the muscle tissue is removed from the individual after delivery of the agent to the individual for at least about 2 months (e.g., at least about any one of 3 months, 6 months, 1 year, 5 years, 10 years or more). In some embodiments, the agent is secreted by the engineered myogenic cells. In some embodiments, the engineered myogenic cells allow delivery of a plurality of agents.

In some embodiments, there is provided a method of delivering an agent to an individual in need thereof, comprising locally (e.g., intramuscularly or subcutaneously) administering to the individual by injection a composition comprising a suspension of engineered myogenic cells, wherein the engineered myogenic cells comprise muscle stem cells, myoblasts, and/or myocytes differentiated from the muscle stem cells or myoblasts, wherein the engineered myogenic cells engraft at the site of administration in the individual, and wherein the engineered myogenic cells are genetically modified to allow delivery of the agent to the individual. In some embodiments, the composition further comprises a carrier, such as a carrier comprising extracellular matrix (ECM) molecules. In some embodiments, the engineered myogenic cells comprise a heterologous nucleic acid sequence encoding the agent. In some embodiments, the agent is delivered locally to the individual. In some embodiments, the agent is delivered systemically to the individual. In some embodiments, the engineered myogenic cells are not produced from an immortalized cell line. In some embodiments, the engineered myogenic cells comprise muscle stem cells, e.g., Pax7⁺ Pax3⁺ MyoD⁻ myogenin⁻, Pax7⁺ Pax3⁻ MyoD⁻ myogenin⁻, and/or Pax7⁻ Pax3⁺ myogenin⁻ cells. In some embodiments, the engineered myogenic cells comprise myoblasts, e.g., Pax7⁻ Pax3⁺ MyoD⁺ myogenin⁻ and/or Pax7⁺ Pax3⁻ MyoD⁺ myogenin⁻ cells. In some embodiments, the engrafted engineered myogenic cells form a muscle tissue in the individual, wherein the muscle tissue allows delivery of the agent to the individual. In some embodiments, the muscle tissue is removed from the individual after delivery of the agent to the individual for at least about 2 months (e.g., at least about any one of 3 months, 6 months, 1 year, 5 years, 10 years or more). In some embodiments, the agent is secreted by the engineered myogenic cells. In some embodiments, the engineered myogenic cells allow delivery of a plurality of agents.

In some embodiments, there is provided a method of delivering an agent to an individual in need thereof, comprising locally (e.g., intramuscularly or subcutaneously) administering to the individual by injection a composition comprising a suspension of engineered myogenic cells, wherein the engineered myogenic cells comprise myoblasts produced from embryonic stem cells (ESC) or induced pluripotent stem cells (iPSC), and/or myocytes differentiated thereof, wherein the engineered myogenic cells engraft at the site of administration in the individual, and wherein the engineered myogenic cells are genetically modified to allow delivery of the agent to the individual. In some embodiments, the composition further comprises a carrier, such as a carrier comprising extracellular matrix (ECM) molecules. In some embodiments, the engineered myogenic cells comprise a heterologous nucleic acid sequence encoding the agent. In some embodiments, the agent is delivered locally to the individual. In some embodiments, the agent is delivered systemically to the individual. In some embodiments, the engineered myogenic cells are not produced from an immortalized cell line. In some embodiments, the myoblasts are Pax7⁻ Pax3⁺ MyoD⁺ myogenin⁻ and/or Pax7⁺ Pax3⁻ MyoD⁺ myogenin⁻ cells. In some embodiments, the engrafted engineered myogenic cells form a muscle tissue in the individual, wherein the muscle tissue allows delivery of the agent to the individual. In some embodiments, the muscle tissue is removed from the individual after delivery of the agent to the individual for at least about 2 months (e.g., at least about any one of 3 months, 6 months, 1 year, 5 years, 10 years or more). In some embodiments, the agent is secreted by the engineered myogenic cells. In some embodiments, the engineered myogenic cells allow delivery of a plurality of agents.

In some embodiments, there is provided a method of delivering an agent to an individual in need thereof, comprising locally (e.g., intramuscularly or subcutaneously) administering to the individual by injection a composition comprising a suspension of engineered myogenic cells, wherein the engineered myogenic cells comprise reprogrammed myogenic cells (e.g., muscle stem cells, myoblasts, or myocytes), wherein the engineered myogenic cells engraft at the site of administration in the individual, and wherein the engineered myogenic cells are genetically modified to allow delivery of the agent to the individual. In some embodiments, the engineered myogenic cells comprise a heterologous nucleic acid sequence encoding the agent. In some embodiments, the composition further comprises a carrier, such as a carrier comprising extracellular matrix (ECM) molecules. In some embodiments, the agent is delivered locally to the individual. In some embodiments, the agent is delivered systemically to the individual. In some embodiments, the engineered myogenic cells are not produced from an immortalized cell line. In some embodiments, the reprogrammed myogenic cells comprise Pax7⁻ Pax3⁺ MyoD⁺ myogenin⁻ and/or Pax7⁺ Pax3⁻ MyoD⁺ myogenin⁻ cells. In some embodiments, the reprogrammed myogenic cells are rejuvenated and/or de-differentiated myogenic cells produced from adult myogenic cells. In some embodiments, the reprogrammed myogenic cells are produced by introducing into the adult myogenic cell a first nucleic acid encoding a LIN28A protein, a second nucleic acid encoding a TERT protein, and a p53 inhibitor (e.g., a third nucleic acid encoding a shRNA targeting p53). In some embodiments, the reprogrammed myogenic cells are transdifferentiated myogenic cells produced from adult somatic cells (e.g., fibroblasts). In some embodiments, the engrafted engineered myogenic cells form a muscle tissue in the individual, wherein the muscle tissue allows delivery of the agent to the individual. In some embodiments, the muscle tissue is removed from the individual after delivery of the agent to the individual for at least about 2 months (e.g., at least about any one of 3 months, 6 months, 1 year, 5 years, 10 years or more). In some embodiments, the agent is secreted by the engineered myogenic cells. In some embodiments, the engineered myogenic cells allow delivery of a plurality of agents.

In some embodiments, there is provided a method of delivering an agent to an individual in need thereof, comprising locally (e.g., intramuscularly or subcutaneously) implanting in the individual a muscle construct comprising engineered myogenic cells, wherein the engineered myogenic cells comprise muscle stem cells, myoblasts, or differentiated cells thereof, wherein the engineered myogenic cells engraft at the site of implantation in the individual and allow delivery of the agent to the individual. In some embodiments, the muscle construct comprises PAX7⁺ myogenic cells. In some embodiments, the muscle construct comprises engineered myogenic cells dispersed in a carrier, such as a carrier comprising ECM molecules and a cell adhesion molecule, e.g., MATRIGEL® and fibrin. In some embodiments, the muscle construct comprises at least about 10⁵ (e.g., at least about 10⁶) of the engineered myogenic cells. In some embodiments, the agent is delivered locally to the individual. In some embodiments, the agent is delivered systemically to the individual. In some embodiments, the engineered myogenic cells are not produced from an immortalized cell line. In some embodiments, the engineered myogenic cells comprise muscle stem cells, e.g., Pax7⁺ Pax3⁺ MyoD⁻ myogenin⁻, Pax7⁺ Pax3⁻ MyoD⁻ myogenin⁻, and/or Pax7⁻ Pax3⁺ MyoD⁻ myogenin⁻ cells. In some embodiments, the engineered myogenic cells comprise myoblasts, e.g., Pax7⁻ Pax3⁺ MyoD⁺ myogenin⁻ and/or Pax7⁺ Pax3⁻ MyoD⁺ myogenin⁻ cells. In some embodiments, the engineered myogenic cells comprise cells produced by differentiating muscle stem cells or myoblasts, such as myocytes, myotubes, and/or myofibers. In some embodiments, the engrafted engineered myogenic cells form a muscle tissue in the individual, wherein the muscle tissue allows delivery of the agent to the individual. In some embodiments, the muscle tissue is removed from the individual after delivery of the agent to the individual for at least about 2 months (e.g., at least about any one of 3 months, 6 months, 1 year, 5 years, 10 years or more). In some embodiments, the agent is secreted by the engineered myogenic cells. In some embodiments, the agent is expressed on the cell surface of the engineered myogenic cells. In some embodiments, the engineered myogenic cells allow delivery of a plurality of agents.

In some embodiments, there is provided a method of delivering an agent to an individual in need thereof, comprising locally (e.g., intramuscularly or subcutaneously) implanting in the individual a muscle construct comprising engineered myogenic cells, wherein the engineered myogenic cells comprise myoblasts produced from ESC or iPSC, or differentiated cells (e.g., myocytes, myotubes, and/or myofibers) thereof, wherein the engineered myogenic cells engraft at the site of implantation in the individual and allow delivery of the agent to the individual. In some embodiments, the muscle construct comprises PAX7⁺ myogenic cells. In some embodiments, the muscle construct comprises engineered myogenic cells dispersed in a carrier, such as a carrier comprising ECM molecules and a cell adhesion molecule, e.g., MATRIGEL® and fibrin. In some embodiments, the muscle construct comprises at least about 10⁵ (e.g., at least about 10⁶) of the engineered myogenic cells. In some embodiments, the agent is delivered locally to the individual. In some embodiments, the agent is delivered systemically to the individual. In some embodiments, the engineered myogenic cells are not produced from an immortalized cell line. In some embodiments, the myoblasts are Pax7⁻ Pax3⁺ MyoD⁺ myogenin⁻ or Pax7⁺ Pax3⁻ MyoD⁺ myogenin⁻ cells. In some embodiments, the engrafted engineered myogenic cells form a muscle tissue in the individual, wherein the muscle tissue allows delivery of the agent to the individual. In some embodiments, the muscle tissue is removed from the individual after delivery of the agent to the individual for at least about 2 months (e.g., at least about any one of 3 months, 6 months, 1 year, 5 years, 10 years or more). In some embodiments, the agent is secreted by the engineered myogenic cells. In some embodiments, the agent is expressed on the cell surface of the engineered myogenic cells. In some embodiments, the engineered myogenic cells allow delivery of a plurality of agents.

In some embodiments, there is provided a method of delivering an agent to an individual in need thereof, comprising locally (e.g., intramuscularly or subcutaneously) implanting in the individual a muscle construct comprising engineered myogenic cells, wherein the engineered myogenic cells comprise reprogrammed myogenic cells (e.g., myoblasts), or differentiated cells (e.g., myocytes, myotubes, and/or myofibers) thereof, wherein the engineered myogenic cells engraft at the site of implantation in the individual and allow delivery of the agent to the individual. In some embodiments, the muscle construct comprises PAX7⁺ myogenic cells. In some embodiments, the muscle construct comprises engineered myogenic cells dispersed in a carrier, such as a carrier comprising ECM molecules and a cell adhesion molecule, e.g., MATRIGEL® and fibrin. In some embodiments, the muscle construct comprises at least about 10⁵ (e.g., at least about 10⁶) of the engineered myogenic cells. In some embodiments, the agent is delivered locally to the individual. In some embodiments, the agent is delivered systemically to the individual. In some embodiments, the engineered myogenic cells are not produced from an immortalized cell line. In some embodiments, the reprogrammed myogenic cells comprise Pax7⁻ Pax3⁺ MyoD⁺ myogenin⁻ and/or Pax7⁺ Pax3⁻ MyoD⁺ myogenin⁻ cells. In some embodiments, the reprogrammed myogenic cells are rejuvenated and/or de-differentiated myogenic cells produced from adult myogenic cells. In some embodiments, the reprogrammed myogenic cells are produced by introducing into the adult myogenic cell a first nucleic acid encoding a LIN28A protein, a second nucleic acid encoding a TERT protein, and a p53 inhibitor (e.g., a third nucleic acid encoding a shRNA targeting p53). In some embodiments, the reprogrammed myogenic cells are transdifferentiated myogenic cells produced from adult somatic cells (e.g., fibroblasts). In some embodiments, the engrafted engineered myogenic cells form a muscle tissue in the individual, wherein the muscle tissue allows delivery of the agent to the individual. In some embodiments, the muscle tissue is removed from the individual after delivery of the agent to the individual for at least about 2 months (e.g., at least about any one of 3 months, 6 months, 1 year, 5 years, 10 years or more). In some embodiments, the agent is secreted by the engineered myogenic cells. In some embodiments, the agent is expressed on the cell surface of the engineered myogenic cells. In some embodiments, the engineered myogenic cells allow delivery of a plurality of agents.

In some embodiments, there is provided a method of delivering an agent to an individual in need thereof, comprising locally (e.g., intramuscularly or subcutaneously) administering to the individual by injection a composition comprising a suspension of engineered myogenic cells, wherein the engineered myogenic cells comprise Pax7⁻ Pax3⁺ MyoD⁺ myogenin⁻ and/or Pax7⁺ Pax3⁻ MyoD⁺ myogenin⁻ cells, and/or myocytes differentiated thereof, wherein the engineered myogenic cells engraft at the site of administration in the individual, wherein the engrafted engineered myogenic cells form a muscle tissue in the individual, and wherein the engineered myogenic cells are genetically modified to allow delivery of the agent to the individual. In some embodiments, the Pax7⁻ Pax3⁺ MyoD⁺ myogenin⁻ and/or Pax7⁺ Pax3⁻ MyoD⁺ myogenin⁻ cells are produced from ESC or iPSC. In some embodiments, the Pax7⁻ Pax3⁺ MyoD⁺ myogenin⁻ and/or Pax7⁺ Pax3⁻ MyoD⁺ myogenin⁻ cells are produced by introducing into the adult myogenic cell a first nucleic acid encoding a LIN28A protein, a second nucleic acid encoding a TERT protein, and a p53 inhibitor (e.g., a third nucleic acid encoding a shRNA targeting p53). In some embodiments, the composition further comprises a carrier, such as a carrier comprising extracellular matrix (ECM) molecules. In some embodiments, the engineered myogenic cells comprise a heterologous nucleic acid sequence encoding the agent. In some embodiments, the agent is delivered locally to the individual. In some embodiments, the agent is delivered systemically to the individual. In some embodiments, the engineered myogenic cells are not produced from an immortalized cell line. In some embodiments, the muscle tissue is removed from the individual after delivery of the agent to the individual for at least about 2 months (e.g., at least about any one of 3 months, 6 months, 1 year, 5 years, 10 years or more). In some embodiments, the agent is secreted by the engineered myogenic cells. In some embodiments, the engineered myogenic cells allow delivery of a plurality of agents.

In some embodiments, there is provided a method of delivering an agent to an individual in need thereof, comprising locally (e.g., intramuscularly or subcutaneously) implanting in the individual a muscle construct comprising engineered myogenic cells, wherein the engineered myogenic cells comprise Pax7⁻ Pax3⁺ MyoD⁺ myogenin⁻ and/or Pax7⁺ Pax3⁻ MyoD⁺ myogenin⁻ cells, wherein the engineered myogenic cells engraft at the site of administration in the individual, wherein the engrafted engineered myogenic cells form a muscle tissue in the individual, and wherein the engineered myogenic cells are genetically modified to allow delivery of the agent to the individual. In some embodiments, the Pax7⁻ Pax3⁺ MyoD⁺ myogenin⁻ and/or Pax7⁺ Pax3⁻ MyoD⁺ myogenin⁻ cells are produced from ESC or iPSC. In some embodiments, the Pax7⁻ Pax3⁺ MyoD⁺ myogenin⁻ and/or Pax7⁺ Pax3⁻ MyoD⁺ myogenin⁻ cells are produced by introducing into the adult myogenic cell a first nucleic acid encoding a LIN28A protein, a second nucleic acid encoding a TERT protein, and a p53 inhibitor (e.g., a third nucleic acid encoding a shRNA targeting p53). In some embodiments, the muscle construct further comprises a carrier, such as a carrier comprising extracellular matrix (ECM) molecules and a cell adhesion molecule, e.g., MATRIGEL® and fibrin. In some embodiments, the engineered myogenic cells comprise a heterologous nucleic acid sequence encoding the agent. In some embodiments, the agent is delivered locally to the individual. In some embodiments, the agent is delivered systemically to the individual. In some embodiments, the engineered myogenic cells are not produced from an immortalized cell line. In some embodiments, the muscle tissue is removed from the individual after delivery of the agent to the individual for at least about 2 months (e.g., at least about any one of 3 months, 6 months, 1 year, 5 years, 10 years or more). In some embodiments, the agent is secreted by the engineered myogenic cells. In some embodiments, the engineered myogenic cells allow delivery of a plurality of agents.

In some embodiments, there is provided a method of delivering an agent to an individual in need thereof, comprising locally (e.g., intramuscularly or subcutaneously) implanting in the individual a muscle construct comprising engineered myogenic cells, wherein the engineered myogenic cells comprise myotubes and/or myofibers produced by culturing Pax7⁻ Pax3⁺ MyoD⁺ myogenin⁻ and/or Pax7⁺ Pax3⁻ MyoD⁺ myogenin⁻ cells in vitro under conditions that allow differentiation of the cells, wherein the engineered myogenic cells engraft at the site of administration in the individual, and wherein the engineered myogenic cells are genetically modified to allow delivery of the agent to the individual. In some embodiments, the Pax7⁻ Pax3⁺ MyoD⁺ myogenin⁻ and/or Pax7⁺ Pax3⁻ MyoD⁺ myogenin⁻ cells are produced from ESC or iPSC. In some embodiments, the Pax7⁻ Pax3⁺ MyoD⁺ myogenin⁻ and/or Pax7⁺ Pax3⁻ MyoD⁺ myogenin⁻ cells are produced by introducing into the adult myogenic cell a first nucleic acid encoding a LIN28A protein, a second nucleic acid encoding a TERT protein, and a p53 inhibitor (e.g., a third nucleic acid encoding a shRNA targeting p53). In some embodiments, the engineered myogenic cells comprise a heterologous nucleic acid sequence encoding the agent. In some embodiments, the agent is delivered locally to the individual. In some embodiments, the agent is delivered systemically to the individual. In some embodiments, the engineered myogenic cells are not produced from an immortalized cell line. In some embodiments, the engrafted engineered myogenic cells form a muscle tissue in the individual, wherein the muscle tissue allows delivery of the agent to the individual. In some embodiments, the muscle tissue is removed from the individual after delivery of the agent to the individual for at least about 2 months (e.g., at least about any one of 3 months, 6 months, 1 year, 5 years, 10 years or more). In some embodiments, the agent is secreted by the engineered myogenic cells. In some embodiments, the engineered myogenic cells allow delivery of a plurality of agents.

In some embodiments, there is provided a method of delivering an agent to an individual in need thereof, comprising locally administering to the individual a composition comprising engineered myogenic cells, wherein the engineered myogenic cells comprise muscle stem cells, myoblasts and/or differentiated cells thereof, wherein the engineered myogenic cells engraft at the site of administration in the individual, wherein the engrafted myogenic cells are genetically modified to secrete the agent, thereby allowing delivery of the agent to the individual. In some embodiments, the engineered myogenic cells comprise a heterologous nucleic acid sequence encoding the agent. In some embodiments, the composition is administered intramuscularly. In some embodiment, the composition is administered subcutaneously. In some embodiments, the agent is delivered locally to the individual. In some embodiments, the agent is delivered systemically to the individual. In some embodiments, the engineered myogenic cells are not produced from an immortalized cell line. In some embodiments, the engineered myogenic cells comprise muscle stem cells, e.g., Pax7⁺ Pax3⁺ MyoD⁻ myogenin⁻, Pax7⁺ Pax3⁻ MyoD⁻ myogenin⁻, and/or Pax7⁻ Pax3⁺ MyoD⁻ myogenin⁻ cells. In some embodiments, the engineered myogenic cells comprise myoblasts, e.g., Pax7⁻ Pax3⁺ MyoD⁺ myogenin⁻ and/or Pax7⁺ Pax3⁻ MyoD⁺ myogenin⁻ cells. In some embodiments, the engineered myogenic cells comprise myoblasts produced from ESC or iPSC. In some embodiments, the engineered myogenic cells comprise reprogrammed myogenic cells (e.g., myoblasts). In some embodiments, the reprogrammed myogenic cells are rejuvenated and/or de-differentiated myogenic cells produced from adult myogenic cells. In some embodiments, the reprogrammed myogenic cells are produced by introducing into the adult myogenic cell a first nucleic acid encoding a LIN28A protein, a second nucleic acid encoding a TERT protein, and a p53 inhibitor (e.g., a third nucleic acid encoding a shRNA targeting p53). In some embodiments, the reprogrammed myogenic cells are transdifferentiated myogenic cells produced from adult somatic cells (e.g., fibroblasts). In some embodiments, the engineered myogenic cells comprise cells produced by differentiating muscle stem cells or myoblasts, such as myocytes, myotubes, and/or myofibers. In some embodiments, the engrafted engineered myogenic cells form a muscle tissue in the individual, wherein the muscle tissue allows delivery of the agent to the individual. In some embodiments, the muscle tissue is removed from the individual after delivery of the agent to the individual for at least about 2 months (e.g., at least about any one of 3 months, 6 months, 1 year, 5 years, 10 years or more). In some embodiments, the engineered myogenic cells allow delivery of a plurality of agents.

The engineered myogenic cells may have anyone or combination of features described in Section IV “Engineered myogenic cells” below. The engineered myogenic cells may be obtained from various sources. In some embodiments, the engineered myogenic cells are autologous. In some embodiments, the engineered myogenic cells are allogeneic. In some embodiments, the engineered myogenic cells are non-immunogenic to the individual. In some embodiments, the engineered myogenic cells are produced from a cell line. In some embodiments, the engineered myogenic cells are not produced from an immortal cell line. In some embodiments, the engineered myogenic cells are produced from primary cells obtained from the individual. In some embodiments, the engineered myogenic cells are produced from primary cells obtained from a donor.

In some embodiments, the method further comprises administering to the individual an effective amount of an immunosuppressant to minimize rejection of the engineered myogenic cells. Examples of immunosuppressive agents include, but are not limited to, methotrexate, cyclophosphamide, cyclosporine, cyclosporin A, chloroquine, hydroxychloroquine, sulfasalazine (sulphasalazopyrine), gold salts, D-penicillamine, leflunomide, azathioprine, anakinra, infliximab (REMICADE), etanercept, TNF alpha, blockers, a biological agent that targets an inflammatory cytokine, and Non-Steroidal Anti-Inflammatory Drug (NSAIDs).

Any of the methods described herein may further comprise one or more steps for in vitro production of the engineered myogenic cells or muscle construct thereof. Any suitable methods for in vitro proliferation and/or differentiation of myogenic cells may be used. See, for example, Chua et al., 2019; and Fukawa et al., 2016. In some embodiments, the method comprises obtaining the engineered myogenic cells (e.g., muscle stem cells, myoblasts, or muscle progenitor cells) from the individual or a donor. In some embodiments, the method comprises any one of the methods of producing reprogrammed myogenic cells from adult myogenic cells as described in Section III “Methods of rejuvenation” below. In some embodiments, the method comprises producing reprogrammed myogenic cells from adult somatic cells (e.g., fibroblasts). In some embodiments, the method comprises culturing the engineered myogenic cells in vitro under conditions that allow proliferation of the engineered myogenic cells. In some embodiments, the method comprises culturing the engineered myoblasts in vitro under conditions that allow proliferation of the myoblasts without differentiation. In some embodiments, the engineered myoblasts are cultured in a proliferation medium comprising DMEM with about 20% FBS. In some embodiments, the method comprises culturing engineered muscle stem cells in vitro under conditions that allow differentiation of the engineered muscle stem cells into myoblasts, myocytes, myotubes, and/or myofibers. In some embodiments, the method comprises culturing engineered myoblasts in vitro under conditions that allow differentiation of the engineered myoblasts into myocytes, myotubes, and/or myofibers. In some embodiments, the method comprises culturing engineered myoblasts in vitro under conditions that allow alignment of the engineered myoblasts into aligned myotubes, and/or myofibers. In some embodiments, the engineered myoblasts are cultured in a differentiation medium comprising DMEM/F12, 2% horse serum, and 1% L-glutamine. In some embodiments, the engineered myogenic cells are cultured in vitro for at least about any one of 3 days, 4 days, 5 days, 6 days, 7 days, 8 days, 9 days, 10 days, 11 days, 12 days, 13 days or more prior to administration to the individual.

Any of the methods described herein may further comprise one or more steps for preparing a muscle construct. Any suitable methods for preparing muscle constructs may be used. See, for example, Velcro-anchored fibrin constructs (e.g., Hinds et al., 2011), and suture-anchored fibrin constructs (e.g., Khodabukus and Baar, 2009), three-dimensional bio-printing of muscle constructs such as by coaxial printing (e.g., Testa, 2018) and using a tissue-derived bio-ink (e.g., Choi, 2019), and culturing muscle progenitor cells on three-dimensional printed molds (e.g., Capel, 2019). In some embodiments, the method comprises culturing engineered muscle progenitor cells (e.g., myoblasts) in vitrounder conditions to produce a muscle construct. In some embodiments, the engineered muscle progenitor cells (e.g., myoblasts) are cultured in a hydrogel carrier, such as a carrier comprising MATRIGEL® and fibrin, to produce a muscle construct. In some embodiments, the engineered muscle progenitor cells (e.g., myoblasts) are cultured on the surface of a hydrogel, such as fibrin, anchored with sutures to produce a muscle construct. In some embodiments, the engineered muscle progenitor cells (e.g., myoblasts) are cultured within a three-dimensional (“3D”) solid mold to produce a pre-shaped muscle construct. In some embodiments, the engineered muscle progenitor cells (e.g., myoblasts) are 3D-printed with ink to produce a defined 3D muscle construct.

In some embodiments, there is provided a method of preparing a non-human meat product for consumption comprising culturing a composition comprising engineered myogenic cells under conditions to produce a muscle construct, wherein the engineered myogenic cells comprise muscle stem cells, myoblasts and/or differentiated cells thereof. In some embodiments, the engineered myogenic cells are genetically modified to allow delivery of an agent.

The composition comprising engineered myogenic cells may be a suspension of cells, or a muscle construct. In some embodiments, the composition is a solution suitable for injection. In some embodiments, the composition is a hydrogel suitable for surgical implantation. Suitable local administration routes include, but are not limited to, intramuscular and subcutaneous administration. The composition may be administered intramuscularly at any suitable location in the body, including, but not limited to, deltoid muscle of the arm, vastus lateralis muscle of the thigh, ventrogluteal muscle of the hip, and dorsogluteal muscles of the buttocks. The composition may be administered subcutaneously at any suitable location in the body, including, but not limited to, outer area of the upper arm, abdomen, front of thigh, upper back, and upper area of buttocks.

In some embodiments, the composition comprises a carrier. In some embodiments, the carrier comprises a cell adhesion molecule, such as fibrin. In some embodiments, the engineered myogenic cells are intermixed with the carrier. In some embodiments, the carrier comprises extracellular matrix molecules. In some embodiments, the carrier comprises MATRIGEL®. In some embodiments, the carrier comprises a cell adhesion molecule, such as fibrin.

In some embodiments, the engrafted myogenic cells or constructs thereof give rise to a muscle tissue at the site of administration in the individual. The muscle tissue stays at the site of administration for an extended period of time (e.g., at least about any one of 2 months, 3 months, 6 months, 1 year, 5 years, 10 years or more). In some embodiments, the muscle tissue is fused to surrounding tissues of the individual. In some embodiments, the muscle tissue is not fused to surrounding tissues of the individual. In some embodiments, the muscle tissue is encapsulated in the hydrogel carrier in vivo. In some embodiments, the muscle tissue is not encapsulated in vivo. In some embodiments, the muscle tissue recruits blood vessels (e.g., capillaries) from surrounding tissues in the individual. In some embodiments, the recruited blood vessels allow systemic delivery of the agent secreted from the muscle tissue. In some embodiments, the agent is delivered locally to cells of surrounding tissues in the individual.

In some embodiments, the muscle tissue persists in the individual in vivo for at least about any one of 7 weeks, 8 weeks, 2 months, 3 months, 6 months, 7 months, 8 months, 9 months, 10 months, 11 months, 1 year, 2 years, 3 years, 5 years, 10 years, 20 years, 30 years, 40 years, 50 years or more. In some embodiments, wherein the individual is a rodent (e.g., mouse), the muscle tissue persists in the individual in vivo for at least about any one of 8 months, 9 months, 10 months, 11 months, 1 year, or more. In some embodiments, the muscle tissue is removed from the individual after the agent is delivered to the individual. In some embodiments, the muscle tissue is not removed from the individual. In some embodiments, the muscle tissue is removed from the individual after the agent has been delivered to the individual for an extended period of time, such as at least about any one of 7 weeks, 8 weeks, 2 months, 3 months, 6 months, 7 months, 8 months, 9 months, 10 months, 11 months, 1 year, 2 years, 3 years, 5 years, 10 years, 20 years, 30 years, 40 years, 50 years or more. In some embodiments, wherein the individual is a rodent (e.g., mouse), the muscle tissue is removed from the individual after the agent has been delivered to the individual for at least about any one of 8 months, 9 months, 10 months, 11 months, 1 year, or more. In some embodiments, the agent is delivered to the individual for at least about any one of 7 weeks, 8 weeks, 2 months, 3 months, 6 months, 7 months, 8 months, 9 months, 10 months, 11 months, 1 year, 2 years, 3 years, 5 years, 10 years, 20 years, 30 years, 40 years, 50 years or more. In some embodiments, wherein the individual is a rodent (e.g., mouse), the agent is delivered to the individual for at least about any one of 8 months, 9 months, 10 months, 11 months, 1 year, or more.

The engineered myogenic cells are genetically modified to allow delivery of an agent to the individual. In some embodiments, the engineered myogenic cells are genetically modified to express the agent. In some embodiments, the engineered myogenic cells are genetically modified to express one or more molecules (e.g., metabolites, DNA, RNA, and/or protein) that allow delivery of the agent. Suitable genetic modifications include, but are not limited to, introduction of mutations (e.g., indel, alternative splicing, substitution, etc.), knock-in or knock-out of genes, and epigenetic modifications. The agent may be native to the engineered myogenic cells, or exogenous to the myogenic cells. In some embodiments, one or more endogenous genes of the engineered myogenic cells may be edited to allow altered expression (e.g., increased expression or inducible expression) of the agent by the engineered myogenic cells. In some embodiments, a heterologous nucleic acid sequence encoding a regulator of the agent is introduced to the engineered myogenic cells to allow altered (e.g., increased expression or inducible expression) expression of the agent by the engineered myogenic cells. In some embodiments, one or more heterologous nucleic acid sequences encoding the agent are introduced to the engineered myogenic cells to allow expression of the agent by the engineered myogenic cells.

The engineered myogenic cells may deliver the agent to the individual in a variety of ways. In some embodiments, the agent is secreted by the engineered myogenic cells. In some embodiments, the agent is expressed on the cell surface of the engineered myogenic cells. In some embodiments, the agent diffuses from the engrafted myogenic cells to the surrounding tissues in the individual. In some embodiments, the agent is actively transported from the engrafted myogenic cells to the surrounding tissues. In some embodiments, the agent is delivered from the engrafted myogenic cells to the surrounding tissues via cell-cell bridges and/or nanotubes. In some embodiments, the agent is delivered via fusion of the engrafted myogenic cells to the surrounding tissue. In some embodiments, the agent is delivered via the vasculature in the muscle tissue formed by the engrafted myogenic cells. In some embodiments, the agent is delivered from the engrafted myogenic cells to the surrounding tissues via cell-cell junctions.

The engrafted engineered myogenic cells or muscle construct allows delivery of a variety of agents, including, but not limited to, small molecules (e.g., metabolites, nutrients, drugs, macrolides), nucleic acids (e.g., DNA such as dsDNA, ssDNA, aptamers, nanostructures, DNAzymes etc., RNA such as mRNA, shRNA, siRNA, lincRNA, microRNA, circRNA, aptamers, ribozymes, tRNA, tmRNA, shRNA, rRNA, guide RNA etc., artificial nucleic acids such as LNA, PNA, morpholinos, modified DNA, modified RNA etc., including therapeutic nucleic acids), peptides, proteins (e.g., growth factors, cytokines, hormones, antibodies, receptors, decoy receptors, receptor ligands, enzymes, or artificial proteins), viruses, extracellular vesicles, vaccines (e.g., DNA, RNA, or protein vaccines), and reporters (e.g., biomarkers or fusion protein with labels). The agents may be naturally occurring, or artificially designed. The agent may be secreted, or expressed on the cell surface. In some embodiments, the engrafted engineered myogenic cells or muscle construct produces the agent constitutively. In some embodiments, production of the agent is inducible.

In some embodiments, the engineered myogenic cells produce a single agent. In some embodiments, the engineered myogenic cells produce a plurality (e.g., any one of 2, 3, 4, 5, 6, 10, 20 or more) of agents.

The composition may be administered to the individual for a single time in the individual's life. In some embodiments, the composition is administered for a plurality (e.g., any one of 2, 3, 4, 5, 6, 10, 20 or more) of times. In some embodiments, a composition comprising a different type of engineered myogenic cells (e.g., myogenic cells capable of delivering a different agent) is administered each time.

The composition may be administered to any individual in need of an agent, especially chronic delivery of an agent. In some embodiments, the individual is a mammal. In some embodiments, the individual is a human. In some embodiments, the individual is a non-human animal model of a disease or condition. In some embodiments, the individual is a non-human primate. In some embodiments, the individual is a rodent, such as mice or rats. In some embodiments, the individual is an infant. In some embodiment, the individual is a child. In some embodiments, the individual is no more than about any one of 18, 17, 16, 15, 14, 13, 12, 11, 10, 9, 8, 7, 6, 5, 4, 3, or 2 years old, 1 year, or 6 months old. In some embodiments, the individual is an adult. In some embodiments, the individual is at least about any one of 2, 3, 4, 5, 6, 7, 8, 9, 10, 11, 12, 13, 14, 15, 16, 17, 18, 20, 25, 30, 35, 40, 45, 50, 55, 60, 70, 80 years old or older.

Further provided are methods of treating or diagnosing a disease or condition in an individual, comprising delivering an agent to the individual using any one of the methods of delivery described herein, wherein the agent treats or diagnoses the disease or condition.

In some embodiments, there is provided a method of treating a disease or condition in an individual, comprising locally administering to the individual a composition comprising engineered myogenic cells, wherein the engineered myogenic cells comprise muscle stem cells, myoblasts and/or differentiated cells thereof, wherein the engineered myogenic cells engraft at the site of administration in the individual, wherein the engineered myogenic cells are genetically modified to allow delivery of a therapeutic agent to the individual, and wherein the therapeutic agent treats the disease or condition. In some embodiments, the engineered myogenic cells comprise a heterologous nucleic acid sequence encoding the therapeutic agent. In some embodiments, the composition is administered intramuscularly. In some embodiment, the composition is administered subcutaneously. In some embodiments, the agent is delivered locally to the individual. In some embodiments, the agent is delivered systemically to the individual. In some embodiments, the engineered myogenic cells are not produced from an immortalized cell line. In some embodiments, the engineered myogenic cells comprise muscle stem cells, e.g., Pax7⁺ Pax3⁺ MyoD⁻ myogenin⁻, Pax7⁺ Pax3⁻ MyoD⁻ myogenin⁻, and/or Pax7⁻ Pax3⁺ MyoD⁻ myogenin⁻ cells. In some embodiments, the engineered myogenic cells comprise myoblasts, e.g., Pax7⁻ Pax3⁺ MyoD⁺ myogenin⁻ and/or Pax7⁺ Pax3⁻ MyoD⁺ myogenin⁻ cells. In some embodiments, the engineered myogenic cells comprise myoblasts produced from ESC or iPSC. In some embodiments, the engineered myogenic cells comprise reprogrammed myogenic cells (e.g., myoblasts). In some embodiments, the reprogrammed myogenic cells are rejuvenated and/or de-differentiated myogenic cells produced from adult myogenic cells. In some embodiments, the reprogrammed myogenic cells are produced by introducing into the adult myogenic cell a first nucleic acid encoding a LIN28A protein, a second nucleic acid encoding a PERT protein, and a p53 inhibitor (e.g., a third nucleic acid encoding a shRNA targeting p53). In some embodiments, the reprogrammed myogenic cells are transdifferentiated myogenic cells produced from adult somatic cells (e.g., fibroblasts). In some embodiments, the engineered myogenic cells comprise cells produced by differentiating muscle stem cells or myoblasts, such as myocytes, myotubes, and/or myofibers. In some embodiments, the engrafted engineered myogenic cells form a muscle tissue in the individual, wherein the muscle tissue allows delivery of the agent to the individual. In some embodiments, the muscle tissue is removed from the individual after delivery of the agent to the individual for at least about 2 months (e.g., at least about any one of 3 months, 6 months, 1 year, 5 years, 10 years or more). In some embodiments, the agent is secreted by the engineered myogenic cells. In some embodiments, the agent is expressed on the cell surface of the engineered myogenic cells. In some embodiments, the engineered myogenic cells allow delivery of a plurality of agents.

The methods of delivery described herein are particularly suitable for use in treating or diagnosing diseases or conditions that need chronic delivery of agents. Exemplary diseases or conditions include, but are not limited to, malnutrition, metabolic diseases or conditions (e.g., diabetes), genetic diseases (e.g., hemophilia), etc.

In some embodiments, there is provided a method of treating diabetes (e.g., type I diabetes) in an individual, comprising locally administering to the individual a composition comprising engineered myogenic cells comprising a nucleotide sequence encoding insulin, wherein the engineered myogenic cells comprise muscle stem cells, myoblasts and/or differentiated cells thereof, wherein the engineered myogenic cells engraft at the site of administration in the individual, and wherein the engineered myogenic cells are genetically modified to allow systemic delivery of insulin to the individual. In some embodiments, the engineered myogenic cells comprise a heterologous nucleic acid sequence encoding insulin.

In some embodiments, there is provided a method of treating hemophilia (e.g., hemophilia B) in an individual, comprising locally administering to the individual a composition comprising engineered myogenic cells comprising a nucleotide sequence encoding Factor IX, wherein the engineered myogenic cells comprise muscle stem cells, myoblasts and/or differentiated cells thereof, and wherein the engineered myogenic cells engraft at the site of administration in the individual, and wherein the engineered myogenic cells are genetically modified to allow systemic delivery of the Factor IX to the individual. In some embodiments, the engineered myogenic cells comprise a heterologous nucleic acid sequence encoding Factor IX.

Also provided are methods of preparing a non-human animal model of a disease or condition, comprising delivering an agent to a non-human animal using any one of the delivery methods described herein, wherein the agent is associated with the disease or condition.

In some embodiments, there is provided a method of preparing a non-human animal model of a disease or condition, comprising locally administering to a non-human animal a composition comprising engineered myogenic cells, wherein the engineered myogenic cells comprise muscle stem cells, myoblasts and/or differentiated cells thereof, wherein the engineered myogenic cells engraft at the site of administration in the non-human animal, wherein the engineered myogenic cells are genetically modified to allow delivery of an agent associated with the disease or condition to the non-human animal, thereby providing the non-human animal model. In some embodiments, the agent is delivered to the bloodstream of the non-human animal. In some embodiments, the agent is a human protein, RNA or metabolite. In some embodiment, the non-human animal is selected from the group consisting of mammals (e.g., pig, cow, sheep, goat, horse, etc.), birds (e.g., chicken, duck, turkey, etc.), fish, invertebrates, reptiles and amphibians. In some embodiments, the non-human animal is a rodent, such as a mouse or a rat. In some embodiments, the non-human animal is a non-human primate.

In some embodiments, there is provided a method of treating a non-human animal model of a disease or condition, comprising locally administering to the non-human animal a composition comprising engineered myogenic cells, wherein the engineered myogenic cells comprise muscle stem cells, myoblasts and/or differentiated cells thereof, wherein the engineered myogenic cells engraft at the site of administration in the non-human animal, wherein the engineered myogenic cells are genetically modified to allow delivery of an agent that treats a disease or condition in the non-human animal. In some embodiments, the agent is delivered to the bloodstream of the non-human animal. In some embodiments, the agent is a human protein, RNA or metabolite. In some embodiment, the non-human animal is selected from the group consisting of mammals (e.g., pig, cow, sheep, goat, horse, etc.), birds (e.g., chicken, duck, turkey, etc.), fish, invertebrates, reptiles and amphibians. In some embodiments, the non-human animal is a rodent, such as a mouse or a rat. In some embodiments, the non-human animal is a non-human primate.

In some embodiments, there is provided a non-human animal model of a disease or condition prepared using any one of the methods described herein. In some embodiments, there is provided a method of screening for a therapeutic agent for treating a disease or condition, comprising administering a therapeutic agent to a non-human animal model of the disease or condition prepared using any one of the methods described herein, and determining efficacy of the therapeutic agent by assessing one or more parameters associated with the disease or condition in the non-human animal model.

### III. Methods of Producing Reprogrammed Myogenic Cells

The present application also provides methods of producing reprogrammed (e.g., rejuvenated and/or de-differentiated) myogenic cells (e.g., muscle stem cells, myoblasts and/or myocytes) from adult myogenic cells. The method comprises introducing into adult myogenic cells a core set of three factors that are highly regulated in fetal development, such as an embryonic regulator, a telomere-associated regulator, and a cell cycle regulator. For example, reprogrammed myogenic cells produced by introducing LIN28A, TERT (e.g., hTERT) and a p53 inhibitor into adult myogenic cells (also referred herein as “LTS myogenic cells”) have significantly enhanced proliferation, differentiation and engraftment capacity, compared to adult myogenic cells. In some embodiments, the reprogrammed myogenic cells are de-differentiated compared to the adult myogenic cells. In some embodiments, the reprogrammed myogenic cells are rejuvenated compared to the adult myogenic cells. In some embodiments, the reprogramed myogenic cells are both de-differentiated and rejuvenated compared to the adult myogenic cells.

In some embodiments, there is provided a method of producing a reprogrammed myogenic cell from an adult myogenic cell, comprising: a) introducing into the adult myogenic cell an embryonic regulator, a telomere-associated regulator, and a cell cycle regulator to provide a transduced myogenic cell; and b) culturing the transduced myogenic cell under conditions to obtain a rejuvenated myogenic cell. In some embodiments, the myogenic cell is a human cell. In some embodiments, the myogenic cell is a muscle stem cell, myoblast, or myocyte. In some embodiments, the reprogrammed myogenic cell is rejuvenated. In some embodiments, the adult myogenic cell expresses high levels of p21^{WAF1}, p16^{INK4a}, and let-7 microRNAs. In some embodiments, the reprogrammed myogenic cell expresses low levels of p21^{WAF1}, p16^{INK4a}, and let-7 microRNAs. In some embodiments, the adult myogenic cell can undergo no more than about 30 population doublings. In some embodiments, the reprogrammed myogenic cell can undergo at least about 90 population doublings. In some embodiments, the reprogrammed myogenic cell is de-differentiated. In some embodiments, the adult myogenic cell does not express PAX3, or expresses a low level of PAX3. In some embodiments, the reprogrammed myogenic cell is PAX3⁺.

The embryonic regulator, the telomere-associated regulator, and the cell cycle regulator can be of any suitable molecular modality, including, protein, nucleic acids (DNA, RNA such as RNAi, miRNA, mRNA, etc.), and small molecules. In some embodiments, the embryonic regulator is an RNA-binding regulator. In some embodiments, the embryonic regulator is a regulator of let-7 miRNA. In some embodiments, the embryonic regulator is LIN28A. In some embodiments, the telomere-associated regulator is a regulator that stabilizes telomeres. In some embodiments, the telomere-associated regulator is shelter in. In some embodiments, the telomere-associated regulator is TERT. In some embodiments, the cell cycle regulator is a p53 inhibitor.

In some embodiments, there is provided a method of producing a reprogrammed myogenic cell from an adult myogenic cell, comprising: a) introducing into the adult myogenic cell LIN28A, TERT, and a p53 inhibitor to provide a transduced myogenic cell; and b) culturing the transduced myogenic cell under conditions to obtain a reprogrammed myogenic cell. In some embodiments, the myogenic cell is a human cell. In some embodiments, the myogenic cell is a muscle stem cell, myoblast, or myocyte. In some embodiments, the reprogrammed myogenic cell is rejuvenated. In some embodiments, the adult myogenic cell expresses high levels of p21^{WAF1}, p16^{INK4a}, and let-7 microRNAs. In some embodiments, the reprogrammed myogenic cell expresses low levels of p21^{WAF1}, p16^{INK4a}, and let-7 microRNAs. In some embodiments, the adult myogenic cell can undergo no more than about 30 population doublings. In some embodiments, the reprogrammed myogenic cell can undergo at least about 90 population doublings. In some embodiments, the reprogrammed myogenic cell is de-differentiated. In some embodiments, the adult myogenic cell does not express PAX3, or expresses a low level of PAX3. In some embodiments, the reprogrammed myogenic cell is PAX3⁺.

In some embodiments, there is provided a method of producing a reprogrammed myogenic cell from an adult myogenic cell, comprising: a) introducing into the adult myogenic cell a first nucleic acid encoding a LIN28A protein, a second nucleic acid encoding a TERT protein, and a third nucleic acid encoding a shRNA targeting p53 to provide a transduced myogenic cell; and b) culturing the transduced myogenic cell under conditions to obtain a reprogrammed myogenic cell. In some embodiments, the myogenic cell is a human cell. In some embodiments, the myogenic cell is a muscle stem cell, myoblast, or myocyte. In some embodiments, the reprogrammed myogenic cell is rejuvenated. In some embodiments, the adult myogenic cell expresses high levels of p21^{WAF1}, p16^{INK4a}, and let-7 microRNAs. In some embodiments, the reprogrammed myogenic cell expresses low levels of p21^{WAF1}, p16^{INK4a}, and let-7 microRNAs. In some embodiments, the adult myogenic cell can undergo no more than about 30 population doublings. In some embodiments, the reprogrammed myogenic cell can undergo at least about 90 population doublings. In some embodiments, the reprogrammed myogenic cell is de-differentiated. In some embodiments, the adult myogenic cell does not express PAX3, or expresses a low level of PAX3. In some embodiments, the reprogrammed myogenic cell is PAX3⁺. In some embodiments, the transduced myogenic cell is cultured in a medium comprising DMEM with about 20% FBS.

In some embodiments, the adult myogenic cells are obtained from a human individual. The adult myogenic cells may be obtained from an individual of at least 18 years old, such as any one of at least about 20, 30, 40, 50, 60, 70, 80 years old or older. In some embodiments, the adult myogenic cells are obtained from healthy donors. In some embodiments, the adult myogenic cells are pre-senescent myogenic cells. In some embodiments, the adult myogenic cells are obtained from an individual having sarcopenia. In some embodiments, the adult myogenic cells are obtained from an individual having cachexia. In some embodiments, the adult myogenic cells are obtained from an individual having cancer. In some embodiments, the adult myogenic cells are derived from a myogenic cell line. In some embodiments, the adult myogenic cells can undergo no more than about any one of 40, 35, 30, 25, 20, 15, 10, 5 or fewer population doublings. In some embodiments, the adult myogenic cells have high expression levels of cell cycle inhibitors, such as p21^{WAF1}, p27^{KIP1}, and p16^{INK4a}, and/or anti-proliferative factors such as let-7 microRNAs, e.g., let-7b/g. In some embodiments, the expression levels (e.g., mRNA expression level or protein expression level) of cell cycle inhibitors (e.g., p21^{WAF1}, p27^{KIP1}, or p16^{INK4a}) and/or anti-proliferative factors (e.g., let-7 microRNAs) in the adult myogenic cells are at least about any one of 2, 3, 5, 10, 20, 50, 100 or more times the expression levels of the corresponding cell cycle inhibitors and/or anti-proliferative factors in the reprogrammed myogenic cells.

A “LIN28A protein” as used herein includes any of the naturally-occurring forms of the Lin-28 Homolog A, or variants thereof that maintain Lin28A activity (e.g. within at least 50%, 80%, 90%, 95%, 96%, 97%, 98%, 99% or 100% activity compared to Lin28A). In some embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Lin28A polypeptide. In some embodiments, the LIN28A protein is human LIN28A protein. In some embodiments, the LIN28A protein is the protein as identified by the NCBI reference sequence NP_078950.1.

A “TERT protein” as used herein includes any of the naturally-occurring forms of the telomerase reverse transcriptase, or variants thereof that maintain TERT activity (e.g. within at least 50%, 80%, 90%, 95%, 96%, 97%, 98%, 99% or 100% activity compared to TERT). In some embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring TERT polypeptide. In some embodiments, the TERT protein is human TERT (hTERT) protein. In some embodiments, the TERT protein is the protein as identified by the NCBI reference sequence NP_937983.2.

A “p53 inhibitor” refers to a molecule that reduces p53 activity and/or expression. “p53” as used herein refers to any isoform of a protein encoded by the human p53 gene or a homologous gene in any non-human species of interest. P53 is also known as tumor protein p53 (TP53), tumor antigen p53, phosphoprotein p53, tumor suppressor p53, antigen NY—CO-13, or transformation-related protein 53 (TRP53). The murine homolog of p53 is encoded by the Trp53 gene. In some embodiments, the p53 inhibitor reduces the activity of a p53 protein. In some embodiments, the p53 inhibitor reduces the expression of a p53 gene. In some embodiments, the p53 inhibitor reduces the activity of a p53 protein and the expression of a p53 gene. Examples of a p53 inhibitor include, but are not limited to nucleic acids, proteins, dominant negative proteins, peptides, oligosaccharides, polysaccharides, lipids, phospholipids, glycolipids, monomers, polymers, small molecules and organic compounds. In some embodiments, the p53 inhibitor is pifithrin. In some embodiments, the p53 inhibitor is a nucleic acid. In some embodiments, the p53 inhibitor is a short hairpin RNA (shRNA). In some embodiments, the p53 inhibitor is a small interfering RNA. In some embodiments, the p53 inhibitor is a protein. In some embodiments, the p53 inhibitor is a dominant negative protein.

The embryonic regulator (e.g., LIN28A), telomere-associated regulator (e.g., TERT) and cell cycle regulator (e.g., p53 inhibitor) are introduced into the adult myogenic cells using any known methods in the art. In some embodiments, the LIN28A, TERT and p53 inhibitor are introduced by transfecting a first nucleic acid encoding a LIN28A protein, a second nucleic acid encoding a TERT (e.g., hTERT) protein, and a third nucleic acid encoding a p53 inhibitor (e.g., shRNA targeting p53) into the adult myogenic cells. The term “transfection” or “transfecting” refers to a process of introducing nucleic acid molecules to a cell by non-viral or viral-based methods. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection, vesicles and electroporation. In some embodiments, the nucleic acid molecules are introduced into a cell using electroporation. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector.

In some embodiments, the nucleic acids encoding a LIN28A protein, a TERT protein and a p53 inhibitor are present in a single vector. In some embodiments, the nucleic acid encoding a LIN28A protein, the nucleic acid encoding a TERT protein, and the nucleic acid encoding a p53 inhibitor are present in different (e.g., 2 or 3) vectors. In some embodiments, the nucleic acids encoding a LIN28A protein, a TERT protein, and a p53 inhibitor are transiently expressed in the transduced myogenic cell. In some embodiments, the nucleic acids encoding a LIN28A protein, a TERT protein, and a p53 inhibitor are integrated in the genome of the transduced myogenic cell. In some embodiments, the nucleic acids are DNA. In some embodiments, the nucleic acids are RNA, such as mRNA.

In some embodiments, the transduced myogenic cell is expanded and subject to a process of selection. A process of selection may include a selection marker introduced into the adult myogenic cell upon transfection. A selection marker may be a gene encoding for a polypeptide with enzymatic activity, an antibiotic resistance gene, or a fluorescent protein. The enzymatic activity includes, but is not limited to, the activity of an acetyltransferase and a phosphotransferase. In some embodiments, the selection marker confers to a transduced myogenic cell the ability to proliferate in the presence of a toxin. Such a toxin typically inhibits cell expansion and/or causes cell death. Examples of such toxins include, but are not limited to, hygromycin, neomycin, puromycin and gentamycin. In some embodiments, the toxin is hygromycin. Through the enzymatic activity of a selection marker, a toxin may be converted to a non-toxin, which no longer inhibits expansion and causes cell death of a transduced myogenic cell. Upon exposure to a toxin, a cell lacking a selection marker may be eliminated and thereby precluded from expansion.

The reprogrammed myogenic cells may be identified by assessing expression of molecular markers such as PAX3, PAX7, MYOD, MYF5, MYOG, ACTA1, MYH1, MYH3, MYH8, and MYH7. The reprogrammed myogenic cells may also be identified by assessing their cellular morphology, and their proliferation and differentiation capacities. In some embodiments, the reprogrammed myogenic cells express higher level of PAX3 and MYOD, and lower levels of ACTA1, MYH1, MYH3, and MYH8, compared to the adult myogenic cells. In some embodiments, the expression levels (e.g., mRNA expression level or protein expression level) of PAX3 in the reprogrammed myogenic cells are at least about any one of 2, 3, 5, 10, 20, 50, 100 or more times the PAX3 expression level in the adult myogenic cells. In some embodiments, the reprogrammed myogenic cells have at least about any one of 60, 70, 75, 80, 85, 90, 95, 100, 110, 120 or more population doublings.

In some embodiments, the reprogrammed myogenic cells have enhanced oxidative phosphorylation (OxPhos), increased levels of mitochondrial reactive oxygen species (mtROS), and enhanced mitohormetic signaling. In some embodiments, the reprogrammed myogenic cells have enhanced stress responses (e.g., unfolded protein response and DNA damage repair). In some embodiments, the reprogrammed myogenic cells have increased glycolysis. In some embodiments, the reprogrammed myogenic cells have increased expression level of HIF1A.

In some embodiments, there is provided a method of activating HIF1A in an adult myogenic cell, comprising introducing to the adult myogenic cell a nucleic acid encoding a LIN28A protein. In some embodiments, the method further comprises introducing to the adult myogenic cell a nucleic acid encoding TERT, and a p53 inhibitor (e.g., a shRNA targeting p53). In some embodiments, the method induces HIF1A-mediated hypoxic response. In some embodiments, the method induces HIF1A-mediated glycolysis.

In some embodiments, there is provided a method of inducing mtROS production in an adult myogenic cell, comprising introducing to the adult myogenic cell a nucleic acid encoding a LIN28A protein. In some embodiments, the method further comprises introducing to the adult myogenic cell a nucleic acid encoding TERT, and a p53 inhibitor (e.g., a shRNA targeting p53).

In some embodiments, there is provided a method of inducing mitohormetic signaling in an adult myogenic cell, comprising introducing to the adult myogenic cell a nucleic acid encoding a LIN28A protein. In some embodiments, the method further comprises introducing to the adult myogenic cell a nucleic acid encoding TERT, and a p53 inhibitor (e.g., a shRNA targeting p53). In some embodiments, the method induces hypoxic stress response. In some embodiments, the method induces glycolytic response. In some embodiments, the method induces DNA damage repair. In some embodiments, the method induces unfolded protein response.

In some embodiments, the transduced myogenic cells or reprogrammed myogenic cells are allowed to proliferate without differentiation in an environment with appropriate cellular nutrients. The environment may be a liquid environment, a solid environment and/or a semisolid environment (e.g., agar, gel etc.). In some embodiments, the transduced myogenic cells or reprogrammed myogenic cells are cultured in a suitable culture medium. The culture medium may support the proliferation and/or differentiation of the cells. In some embodiments, the culture medium comprises DMEM with about 20% FBS. In some embodiments, the culture medium comprises DMEM/F12, about 2% horse serum, and about 1% L-glutamine. In some embodiments, the transduced myogenic cells or reprogrammed myogenic cells are cultured in vitro for at least about any one of 1, 2, 3, 4, 5, 6, 7, 8, 9, 10, 11, 12, 13, 14 or more days. In some embodiments, the transduced myogenic cells or reprogrammed myogenic cells are cultured in vitro for no more than about any one of 14, 13, 12, 11, 10, 9, 8, 7, 6, 5, 4, 3, 2, or 1 day.

In some embodiments, there is provided a reprogrammed (e.g., rejuvenated and/or de-differentiated) myogenic cell prepared according to any one of the methods of producing reprogrammed myogenic cells described herein. In some embodiments, there is provided a reprogrammed myogenic cell comprising an adult myogenic cell (e.g., myoblast) comprising a first heterologous nucleic acid sequence encoding a LIN28A protein, a second heterologous nucleic acid sequence encoding a TERT protein, and a third heterologous nucleic acid sequence encoding a p53 inhibitor (e.g., a shRNA targeting p53). In some embodiments, the reprogrammed myogenic cell does not comprise heterologous nucleic acids encoding other regulatory factors in embryonic or fetal development.

Also provided herein are methods of producing a myocyte, myotube, myofiber, or a muscle construct using any one of the reprogrammed myogenic cells described herein. The method comprises culturing are programmed myogenic cell under suitable conditions to induce differentiation of the reprogrammed myogenic cell. For example, in some embodiments, the method comprises culturing the reprogrammed myogenic cell (e.g., myoblast) in a growth medium comprising DMEM/F12, about 2% horse serum, and about 1% L-glutamine.

The reprogrammed myogenic cells described herein may be used in any one of the methods of delivery described in Section II above. In some embodiments, there is provided a method of delivering an agent to an individual in need thereof, comprising locally administering to the individual a composition comprising engineered myogenic cells, wherein the engineered myogenic cells comprise reprogrammed myogenic cells (e.g., muscle stem cells, myoblasts, and/or myocytes) and/or differentiated cells (e.g., myotubes, and/or myofibers) thereof, wherein the reprogrammed myogenic cells are produced by introducing into an adult myogenic cell an embryonic regulator (e.g., a first nucleic acid encoding a LIN28A protein), a telomere-associated regulator (e.g., a second nucleic acid encoding a TERT protein such as hTERT), and a cell cycle regulator (e.g., a p53 inhibitor such as a third nucleic acid encoding a shRNA targeting p53), wherein the engineered myogenic cells engraft at the site of administration in the individual, and wherein the engineered myogenic cells are genetically modified to allow delivery of the agent to the individual. In some embodiments, the engineered myogenic cells comprise a heterologous nucleic acid sequence encoding the agent. In some embodiments, the composition is administered intramuscularly. In some embodiment, the composition is administered subcutaneously. In some embodiments, the agent is delivered locally to the individual. In some embodiments, the agent is delivered systemically to the individual. In some embodiments, the engrafted engineered myogenic cells form a muscle tissue in the individual, wherein the muscle tissue allows delivery of the agent to the individual. In some embodiments, the muscle tissue is removed from the individual after delivery of the agent to the individual for at least about 2 months (e.g., at least about any one of 3 months, 6 months, 1 year, 5 years, 10 years or more). In some embodiments, the agent is secreted by the engineered myogenic cells. In some embodiments, the engineered myogenic cells allow delivery of a plurality of agents.

The reprogrammed myogenic cells may also be used for treating or diagnosing a disease or condition in an individual by allowing delivery of an agent to the individual. The reprogrammed myogenic cells are also useful for treating an individual in need of tissue repair, or a muscle disease or condition, such as sarcopenia or cachexia.

In some embodiments, there is provided a method of treating a muscle disease or condition in an individual in need thereof, comprising administering to the individual an effective amount of the reprogrammed myogenic cells or differentiated cells thereof (e.g., myocytes, myotubes and/or myofibers), wherein the reprogrammed myogenic cells are produced using any one of the methods of producing reprogrammed myogenic cells described herein. In some embodiments, the method further comprising obtaining an adult myogenic cell from the individual, and producing the reprogrammed myogenic cells using the adult myogenic cell. In some embodiments, the disease or condition is sarcopenia or cachexia.

Generally, dosages, schedules, and routes of administration of the reprogrammed myogenic cells or differentiated cells thereof may be determined according to the size and condition of the individual, and according to standard pharmaceutical practice. Exemplary routes of administration include intravenous, intra-arterial, intraperitoneal, intramuscular, subcutaneous, or transdermal. In some embodiments, the reprogrammed myogenic cells or differentiated cells thereof are administered subcutaneously. In some embodiments, the reprogrammed myogenic cells or differentiated cells thereof are administered intramuscularly. In some embodiments, the reprogrammed myogenic cells or differentiated cells thereof are administered by injection. In some embodiments, the reprogrammed myogenic cells or differentiated cells thereof are administered by surgical implantation.

The dose of the cells administered to an individual may vary according to, for example, the particular type of cells being administered, the route of administration, and the particular type of disease or conditions being treated. The amount should be sufficient to produce a desirable response, such as a therapeutic response against the disease or condition, but without severe toxicity or adverse events. In some embodiments, the reprogrammed myogenic cells or differentiated cells thereof are administered at a therapeutically effective amount.

### IV. Engineered Myogenic Cells

The present application also provides engineered myogenic cells (including reprogrammed myogenic cells) or compositions thereof that can be used in the methods of delivery or methods of treatment described herein. Also provided are muscle constructs comprising any of the engineered myogenic cells (including reprogrammed myogenic cells) described herein.

One aspect of the present application provides a non-human meat product for consumption comprising any one of the engineered myogenic cells (including reprogrammed myogenic cells) described herein. In some embodiments, the engineered myogenic cells are from animals selected from the group consisting of mammals (e.g., pig, cow, sheep, goat, horse, etc.), birds (e.g., chicken, duck, turkey, etc.), fish, invertebrates, reptiles and amphibians.

In some embodiments, the engineered myogenic cells can proliferate and/or differentiate to give rise to a skeletal muscle tissue. In some embodiments, the engineered myogenic cells can proliferate and/or differentiate to give rise to a smooth muscle tissue. In some embodiments, the engineered myogenic cells can proliferate and/or differentiate to give rise to a cardiac muscle tissue.

In some embodiments, the engineered myogenic cells have high engraftment efficiency. Engraftment efficiency may be measured using known methods in the art, including, e.g., immunostaining of source-specific markers of the engineered myogenic cells in tissue samples obtained from the individual that receives the engineered myogenic cell transplant. For example, engraftment efficiency can be determined by quantifying the percentage area or percentage of cells that express certain graft-specific markers, such as human lamin A (for human engineered myogenic cells) or a fluorescent protein (e.g., GFP), and determining the peak or average percentage value across different sample sections.

In some embodiments, the engineered myogenic cells comprise muscle progenitor cells (such as muscle stem cells or myoblasts) having high proliferative capacity, or differentiated cells thereof. Proliferative capacity can be assessed by determining population doublings, which refers to the total number of times the cells in a population have doubled since their primary isolation in vitro. This is usually an estimate rounded off to the nearest whole number. A formula to use for the calculation of population doublings is as follows: n=3.32 (log UCY−log 1)+X, where n=the final population doubling number at end of a given subculture, UCY=the cell yield at that point, l=the cell number used as inoculum to begin that subculture, and X=the doubling level of the inoculum used to initiate the subculture being quantitated. In some embodiments, the engineered myogenic cells are muscle progenitor cells having population doublings of at least about 50, 55, 60, 65, 70, 75, 80, 85, 90, 95, 100 or more.

In some embodiments, the engineered myogenic cells comprise PAX7⁺ cells. In some embodiments, the engineered myogenic cells comprise PAX3⁺ cells. In some embodiments, at least about any one of 5%, 10%, 20%, 30%, 40%, 50%, 60%, 70%, 80%, 90% or higher percentage of the engineered myogenic cells are PAX7⁺ cells or PAX3⁺ cells. In some embodiments, PAX7⁺ cells express PAX7 at a level (mRNA or protein level) at least about any one of 2, 5, 10, 15, 20, 50, 100, 200, 500, 1000, 2000, 5000, 10000 fold or higher than terminally differentiated myofiber cells. In some embodiments, PAX3⁺ cells express PAX3 at a level (mRNA or protein level) at least about any one of 2, 5, 10, 15, 20, 50, 100, 200, 500, 1000, 2000, 5000, 10000 fold or higher than terminally differentiated myofiber cells.

Suitable myogenic cells include, but are not limited to muscle stem cells (e.g., satellite cells), embryonic and fetal myoblasts, myoblasts produced from ESC or iPSC, reprogrammed myogenic cells (e.g., rejuvenated and/or de-differentiated myogenic cells) such as those described in Section III below or transdifferentiated myogenic cells, or differentiated cells produced from such muscle stem cells, myoblasts, or reprogrammed myogenic cells.

In some embodiments, the engineered myogenic cells comprise muscle stem cells. In some embodiments, the engineered myogenic cells consist of (or consist essentially of) muscle stem cells. In some embodiments, the muscle stem cells are naturally occurring muscle stem cells, such as satellite cells. In some embodiments, the muscle stem cells are reprogrammed (e.g., rejuvenated and/or de-differentiated) muscle stem cells produced from adult myogenic cells (e.g., myoblasts). In some embodiments, the muscle stem cells are transdifferentiated muscle stem cells produced from adult somatic cells (e.g., fibroblasts). In some embodiments, the muscle stem cells comprise Pax7⁺ Pax3⁺ MyoD⁻ myogenin⁻ cells, Pax7⁺ Pax3⁻ MyoD⁻ myogenin⁻ cells, Pax7⁻ Pax3⁺ MyoD⁻ myogenin⁻ cells, or combinations thereof. In some embodiments, the muscle stem cells are Pax7⁺ Pax3⁺ MyoD⁻ myogenin⁻ cells. In some embodiments, the muscle stem cells are Pax7⁺ Pax3⁻ MyoD⁻ myogenin⁻ cells. In some embodiments, the muscle stem cells are Pax7⁻ Pax3⁺ MyoD⁻ myogenin⁻ cells.

Muscle stem cells may be obtained using known methods in the art. See, for example, by culturing isolated muscle stem cells from young individuals and culturing the muscle stem cells by differential adhesion (e.g., Skuk, 2010), by FACS sorting of adult muscle stem cells (e.g., Conboy, 2010), and by preparing muscle stem cells from ESC or iPSC (e.g., Darabi, 2008; Borchin, 2013; Shelton, 2016), which are incorporated herein by reference in their entirety.

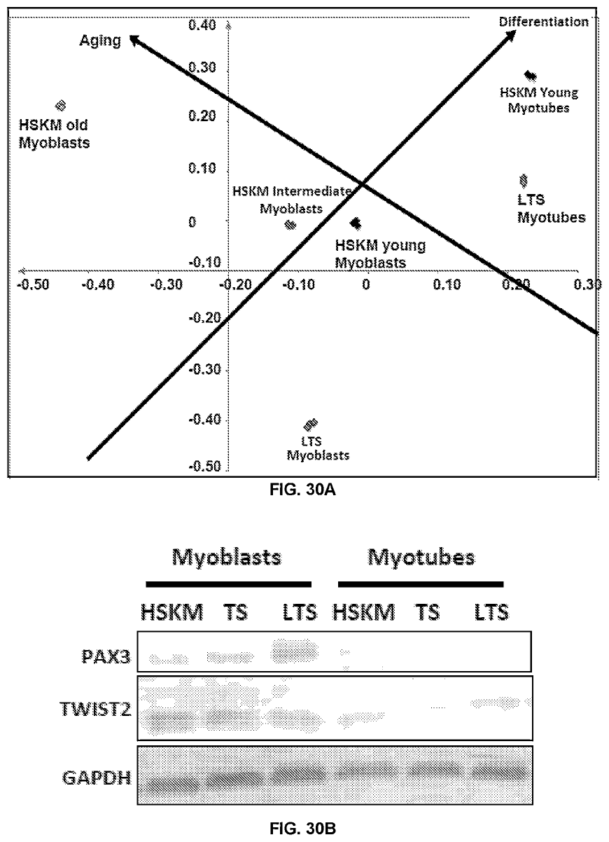

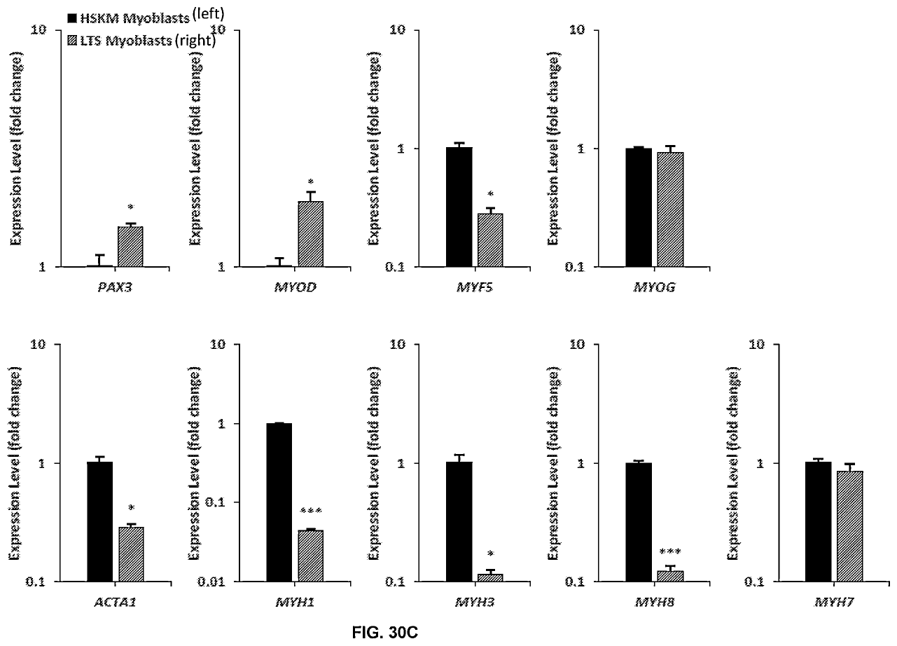

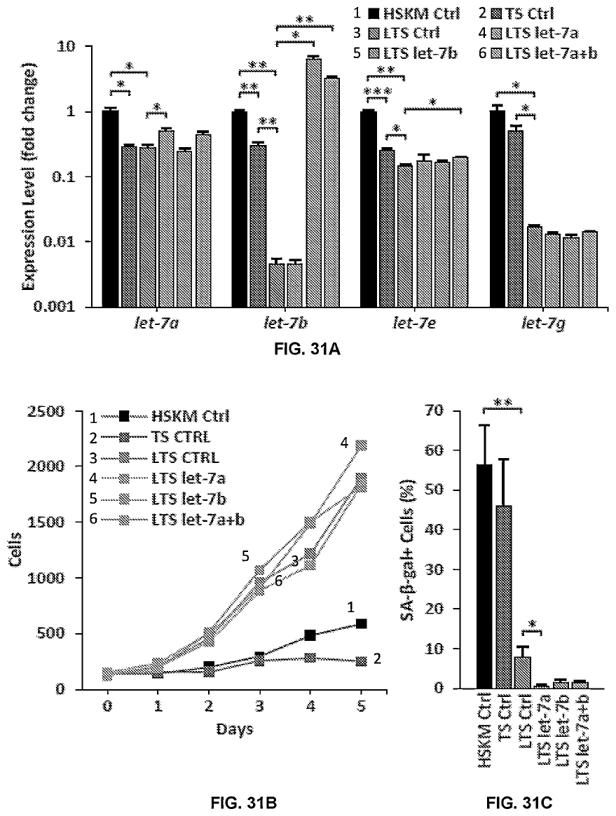

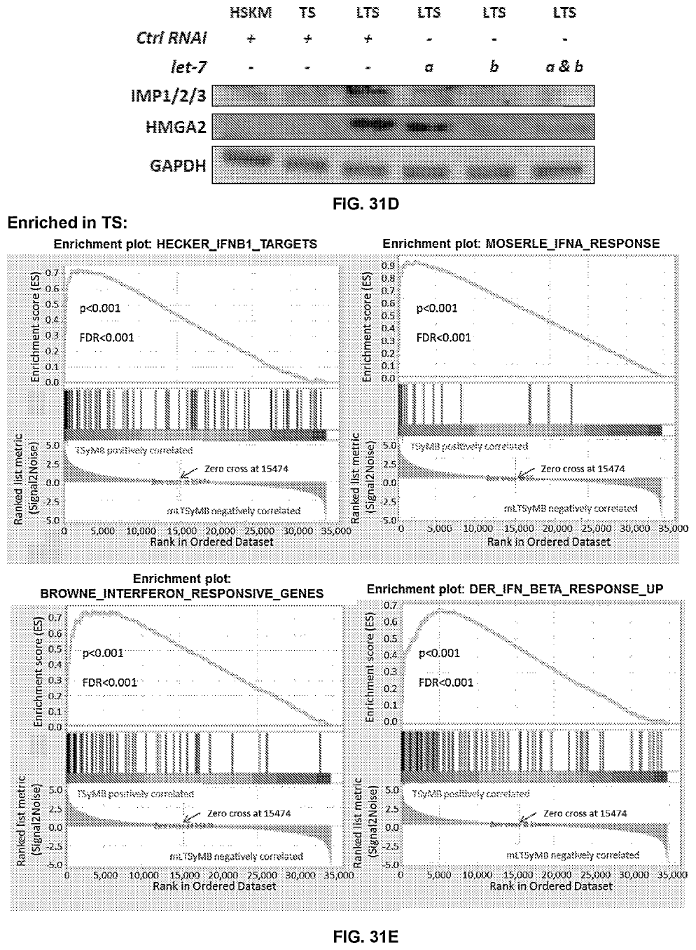

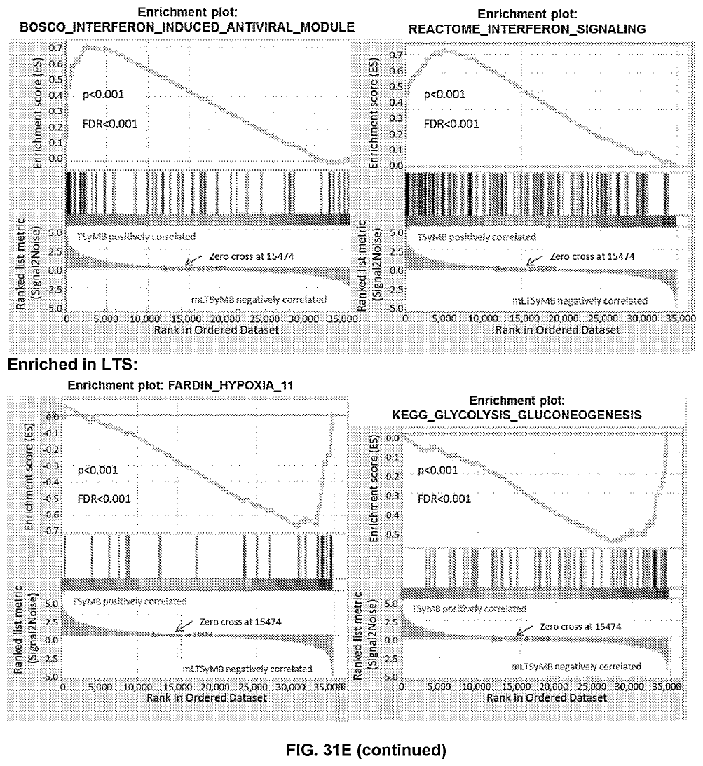

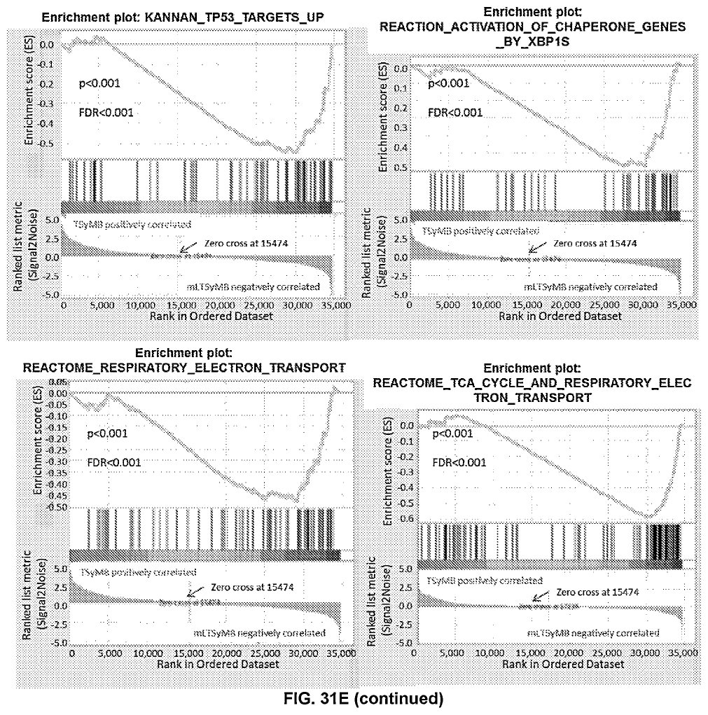

In some embodiments, the engineered myogenic cells comprise myoblasts. In some embodiments, the engineered myogenic cells comprise muscle stem cells and myoblasts. In some embodiments, the engineered myogenic cells consist of (or consist essentially of) myoblasts. In some embodiments, the myoblasts comprise embryonic myoblasts, fetal myoblasts, myoblasts produced from ESC, myoblasts produced from iPSC, reprogrammed myogenic cells, or combinations thereof. In some embodiments, the myoblasts are not primary adult myoblasts. In some embodiments, the myoblasts are embryonic or fetal myoblasts. In some embodiments, the myoblasts are produced from ESC. In some embodiments, the myoblasts are produced from iPSC. In some embodiments, the myoblasts are reprogrammed (e.g., rejuvenated and/or de-differentiated) myoblasts produced from adult myogenic cells, such as LTS myogenic cells, i.e., adult myogenic cells transduced with one or more nucleic acids encoding a LIN28A protein, a PERT protein, and a shRNA targeting p53. In some embodiments, the myoblasts are rejuvenated myoblasts produced from adult myoblasts. In some embodiments, the myoblasts are transdifferentiated myoblasts produced from adult somatic cells (e.g., fibroblasts). In some embodiments, the myoblasts comprise Pax7⁻ Pax3⁺ MyoD⁺ myogenin⁻ cells, Pax7⁺ Pax3⁻ MyoD⁺ myogenin⁻ cells, or combinations thereof. In some embodiments, the myoblasts are Pax7⁻ Pax3⁺ MyoD⁺ myogenin⁻ cells. In some embodiments, the myoblasts are Pax7⁺ Pax3⁻ MyoD⁺ myogenin⁻ cells. In some embodiments, the myoblasts have the gene expression signature as shown in FIG. 30C or FIG. 31E. In some embodiments, the myoblasts have high expression levels of PAX3 and/or MYOD compared to adult myoblasts, and low expression levels of MYF5, ACTA1, MYH1, MYH3, and/or MYH8 compared to adult myoblasts. In some embodiments, the myoblasts have upregulated expression levels in hypoxia targets, glycolysis genes, mitochondrial oxidative phosphorylation (OxPhos) genes, genes in XBP1S-mediated unfolded protein response, and genes in p53-mediated DNA damage repair (DDR) response. In some embodiments, the myoblasts have downregulated expression levels in interferon response targets.

Myoblasts may be produced from ESC or iPSC using known methods in the art. See, for example, Darabi, 2008; Borchin, 2013; Shelton, 2016, which are incorporated herein by reference in their entirety. Reprogrammed (e.g., rejuvenated and/or de-differentiated) myoblasts may be produced using any one of the methods described in section III below. Additionally, muscle progenitor cells such as muscle stem cells and myoblasts may be produced by direct reprogramming of adult somatic cells using myogenic transcription factor(s) and/or small molecule drugs, for example, transdifferentiation of mouse fibroblasts by transient expression of MyoD in combination with GSK3β inhibitor (e.g., CHIR99021), TGF-β inhibitor (e.g., RepSox), and/or cAMP agonist (e.g., Forskolin). See, Bar-Nur, 2018, the contents of which are incorporated herein by reference in its entirety. Myoblasts may be proliferated without differentiation by culturing myoblasts under suitable conditions, for example in a proliferation medium comprising DMEM and about 20% FBS, and passaged before about 80% confluency each time.

In some embodiments, the engineered myogenic cells comprise myocytes produced by differentiating muscle stem cells or myoblasts according to any one of the muscle stem cells or myoblasts described above in vitro. In some embodiments, the engineered myogenic cells comprise muscle stem cells and myocytes produced from the muscle stem cells. In some embodiments, the engineered myogenic cells comprise myoblasts and myocytes produced from the myoblasts. In some embodiments, the engineered myogenic cells comprise muscle stem cells, myoblasts, and myocytes. In some embodiments, the engineered myogenic cells consist of (or consist essentially of) myocytes. In some embodiments, the engineered myogenic cells consist of (or consist essentially of) myocytes produced from embryonic myoblasts, fetal myoblasts, myoblasts produced from ESC, myoblasts produced from iPSC, reprogrammed myogenic cells, or combinations thereof. In some embodiments, the myocytes are produced by differentiating myoblasts produced from iPSC. In some embodiments, the myocytes are rejuvenated myocytes produced from adult myocytes. In some embodiments, the myocytes are produced by differentiating reprogrammed muscle progenitor cells (e.g., myoblasts) produced from adult muscle progenitor cells (e.g., myoblasts), such as LTS muscle progenitor cells. In some embodiments, the myocytes are produced by differentiating transdifferentiated muscle progenitor cells (e.g., myoblasts) produced from adult somatic cells (e.g., fibroblasts). In some embodiments, the myocytes are Pax3⁻ Pax7⁻ MyoD⁺ myogenin⁺ cells.

Myocytes may be produced from myoblasts by culturing the myoblasts under suitable conditions. For example, myoblasts may be allowed to reach 100% confluency and cultured in a differentiation medium comprising DMEM/F12, about 2% horse serum and about 1% L-glutamine for about 2 days.

In some embodiments, the engineered myogenic cells comprise myotubes produced by differentiating muscle stem cells or myoblasts according to any one of the muscle stem cells or myoblasts described above in vitro. In some embodiments, the engineered myogenic cells comprise muscle stem cells and myotubes produced from the muscle stem cells. In some embodiments, the engineered myogenic cells comprise myoblasts and myotubes produced from the myoblasts. In some embodiments, the engineered myogenic cells comprise myoblasts, myocytes produced from the myoblasts, and myotubes produced from the myoblasts. In some embodiments, the engineered myogenic cells comprise muscle stem cells, myoblasts, myocytes and myotubes. In some embodiments, the engineered myogenic cells consist of (or consist essentially of) myotubes. In some embodiments, the engineered myogenic cells consist of (or consist essentially of) myotubes produced from embryonic myoblasts, fetal myoblasts, myoblasts produced from ESC, myoblasts produced from iPSC, reprogrammed myogenic cells, or combinations thereof. In some embodiments, the myotubes are produced by differentiating myoblasts produced from iPSC. In some embodiments, the myotubes are produced by differentiating reprogrammed myogenic cells (e.g., myoblasts) produced from adult myogenic cells (e.g., myoblasts), such as LTS myogenic cells. In some embodiments, the myotubes are produced by differentiating transdifferentiated muscle progenitor cells (e.g., myoblasts) produced from adult somatic cells (e.g., fibroblasts). In some embodiments, the myotubes are myogenin⁺ MRF4+ MHC+ Desmin+ cells. “MHC” refers to myosin heavy chain.

In some embodiments, the engineered myogenic cells comprise myofibers produced by differentiating muscle stem cells or myoblasts according to any one of the muscle stem cells or myoblasts described above in vitro. In some embodiments, the engineered myogenic cells comprise muscle stem cells and myofibers produced from the muscle stem cells. In some embodiments, the engineered myogenic cells comprise myoblasts and myofibers produced from the myoblasts. In some embodiments, the engineered myogenic cells comprise myotubes and myofibers produced from the myoblasts. In some embodiments, the engineered myogenic cells comprise myoblasts, myotubes produced from the myoblasts, and myofibers produced from the myoblasts. In some embodiments, the engineered myogenic cells comprise myoblasts, myocytes produced from the myoblasts, myotubes produced from the myoblasts, and myofibers produced from the myoblasts. In some embodiments, the engineered myogenic cells comprise muscle stem cells, myoblasts, myocytes, myotubes and myofibers. In some embodiments, the engineered myogenic cells consist of (or consist essentially of) myofibers. In some embodiments, the engineered myogenic cells consist of (or consist essentially of) myofibers produced from embryonic myoblasts, fetal myoblasts, myoblasts produced from ESC, myoblasts produced from iPSC, reprogrammed myogenic cells, or combinations thereof. In some embodiments, the myofibers are produced by differentiating myoblasts produced from iPSC. In some embodiments, the myofibers are produced by differentiating reprogrammed myogenic cells (e.g., myoblasts) produced from adult myogenic cells (e.g., myoblasts), such as LTS myogenic cells. In some embodiments, the myofibers are produced by differentiating transdifferentiated muscle progenitor cells (e.g., myoblasts) produced from adult somatic cells (e.g., fibroblasts). In some embodiments, the myofibers are multi-nucleated cells with α-actinin⁺striation. In some embodiments, the myofibers are adult fast MHC⁺ cells. In some embodiments, the engineered myogenic cells have a gene expression signature as shown in FIG. 12.

Myotubes and myofibers may be produced from myoblasts by culturing the myoblasts under suitable conditions. For example, myoblasts may be allowed to reach about 100% confluency and cultured in a differentiation medium comprising DMEM/F12, about 2% horse serum and about 1% L-glutamine for about 13 days.

The engineered myogenic cells are genetically modified to allow delivery of an agent of interest to an individual receiving the engineered myogenic cells. In some embodiments, the engineered myogenic cells are genetically modified to express the agent. In some embodiments, the engineered myogenic cells are genetically modified to express one or more molecules (e.g., metabolites, DNA, RNA, and/or protein) that allow delivery of the agent. Suitable genetic modifications include, but are not limited to, introduction of mutations (e.g., indel, alternative splicing, substitution, etc.), knock-in or knock-out of genes, and epigenetic modifications. The agent may be native to the engineered myogenic cells, or exogenous to the myogenic cells. In some embodiments, the engineered myogenic cells comprise one or more heterologous nucleic acid sequences that encode the agent(s). In some embodiments, the engineered myogenic cells comprise a heterologous nucleic acid sequence encoding the agent, wherein the heterologous nucleic acid sequence is operably linked to a promoter. In some embodiments, the promoter is a constitutive promoter. In some embodiments, the promoter is an inducible promoter. In some embodiments, the engineered myogenic cells comprise one or more heterologous nucleic acid sequences encoding a genetic circuit that synthesizes the agent. In some embodiments, one or more endogenous genes of the engineered myogenic cells are edited to allow production of the agent. In some embodiments, one or more endogenous genes of the engineered myogenic cells may be edited to allow altered expression (e.g., increased expression or inducible expression) of the agent by the engineered myogenic cells. In some embodiments, a heterologous nucleic acid sequence encoding a regulator of the agent is introduced to the engineered myogenic cells to allow altered (e.g., increased expression or inducible expression) expression of the agent by the engineered myogenic cells.

In some embodiments, the engineered myogenic cells produce a single agent. In some embodiments, the engineered myogenic cells produce a plurality (e.g., any one of 2, 3, 4, 5, 6, 10, 20 or more) of agents.

Myogenic cells can be engineered to produce a variety of agents, including, but not limited to, metabolites, nutrients (e.g., vitamins), small molecule drugs, biologics, reporters and combinations thereof. Biologics include, but are not limited to, oligopeptides, DNA (e.g., dsDNA, ssDNA, aptamers, nanostructures, DNAzymes etc.), RNA (e.g., mRNA, shRNA, siRNA, lincRNA, microRNA, circRNA, aptamers, ribozymes, tRNA, tmRNA, shRNA, rRNA, guide RNA etc.), artificial nucleic acids (e.g., LNA, PNA, morpholinos, modified DNA, modified RNA etc.), proteins (e.g., growth factors, cytokines, hormones, antibodies, receptors, decoy receptors, receptor ligands, enzymes, or artificial proteins), viruses, extracellular vesicles, vaccines (e.g., DNA, RNA or protein vaccines), and reporters (e.g., biomarkers, fusion proteins with labels, etc.). In some embodiments, the agent is insulin, or factor IX. The agents may be naturally occurring, or artificially designed. In some embodiments, the agent is secreted by the engineered myogenic cells. In some embodiments, the agent is expressed on the cell surface of the engineered myogenic cells. In some embodiments, the agent is expressed intracellularly.

The heterologous nucleic acid sequences for producing one or more agent(s) or for producing one or more molecules that allow for delivery of the one or more agent(s) can be introduced into the myogenic cells using one or more vectors. Numerous vectors are known in the art including, but not limited to, linear polynucleotides, polynucleotides associated with ionic or amphiphilic compounds, plasmids, and viruses. In general, a suitable vector contains an origin of replication functional in at least one organism, a promoter sequence, convenient restriction endonuclease sites, and one or more selectable markers. The term “vector” should also be construed to include non-plasmid and non-viral compounds, which facilitate transfer of nucleic acid into cells, such as, for example, polylysine compounds, liposomes, and the like.

In some embodiments, the vector is a viral vector. Examples of viral vectors include, but are not limited to, adenoviral vectors, adeno-associated virus vectors, lentiviral vector, retroviral vectors, vaccinia vector, herpes simplex viral vector, and derivatives thereof. Viral vector technology is well known in the art and is described, for example, in Sambrook et al. (2001, Molecular Cloning: A Laboratory Manual, Cold Spring Harbor Laboratory, New York), and in other virology and molecular biology manuals.

The engineered myogenic cells may be obtained from any suitable source. In some embodiments, the engineered myogenic cells are mammalian cells. In some embodiments, the engineered myogenic cells are human cells. In some embodiments, the engineered myogenic cells are produced from a cell line. In some embodiments, the engineered myogenic cells are not produced from a cell line. In some embodiments, the engineered myogenic cells are not produced from an immortal cell line. In some embodiments, the engineered myogenic cells are primary cells. In some embodiments, the engineered myogenic cells are autologous. In some embodiments, the engineered myogenic cells are obtained from the individual in need of agent delivery or treatment using the engineered myogenic cells. In some embodiments, the engineered myogenic cells are allogeneic. In some embodiments, the engineered myogenic cells are obtained from a healthy donor. In some embodiments, the engineered myogenic cells are non-immunogenic to the individual. In some embodiments, the engineered myogenic cells are produced from universal stem cells or universal muscle progenitor cells (e.g., myoblasts), which have been genetically edited to repress or ablate genes required for immune recognition.

Also provided herein are compositions, such as pharmaceutical compositions, comprising engineered myogenic cells or reprogrammed myogenic cells.

In some embodiments, the composition is suitable for local administration to the individual. In some embodiments, the composition is a suspension of the engineered myogenic cells, or a muscle construct comprising the engineered myogenic cells. In some embodiments, the composition is a suspension of muscle stem cells, myoblasts, and/or myocytes differentiated from the muscle stem cells or myoblasts. In some embodiments, the composition is a muscle construct comprising muscle stem cells and/or myoblasts. In some embodiments, the composition is a muscle construct comprising differentiated cells produced from muscle stem cells and/or myoblasts. In some embodiments, the composition is a muscle construct comprising myotubes and/or myofibers differentiated from myoblasts that are produced from ESC or iPSC, or from reprogrammed (e.g., rejuvenated and/or de-differentiated, or trans differentiated) myogenic cells. Cell suspensions may be suitable for injection, such as intramuscular or subcutaneous injection. Muscle constructs may be suitable for local implantation, such as implantation in muscle or adipose tissue.

In some embodiments, the composition comprises a carrier. In some embodiments, the carrier is liquid. In some embodiments, the carrier is a semi-liquid. In some embodiments, the carrier is semi-solid. In some embodiments, the carrier is in a gel state. In some embodiments, the carrier is a culturing medium, such as a medium suitable for cell proliferation and/or differentiation. In some embodiments, the carrier is a hydrogel carrier. In some embodiments, the carrier is a biocompatible carrier. In some embodiments, the carrier comprises extracellular matrix (ECM) molecules, including, but not limited to, laminin, collagen, entactin, heparin sulfate proteoglycan, growth factors, collagenases, and/or plasminogen activators. In some embodiments, the carrier comprises a cell adhesion molecule, such as fibrin. In some embodiments, the carrier comprises MATRIGEL®. In some embodiments, the carrier comprises fibrin, such as fibrin polymer prepared by mixing a composition comprising fibrinogen with thrombin. In some embodiments, the composition comprises an anti-fibrinolytic agent, which inhibits protease-mediated degradation of the fibrin matrix. Suitable anti-fibrinolytic agents include, but are not limited to, aprotinin, epsilon amino-caproic acid, tranexamic acid, and protease inhibitors such as soybean trypsin inhibitor, etc.

In some embodiments, the amount of the carrier in the composition is no more than about any one of 95%, 90%, 85%, 80%, 75%, 70%, 60%, 50%, 40%, 30%, 20%, 10% (weight/weight) or less. In some embodiments, the carrier comprises at least about 50%, 55%, 60%, 65%, 70%, 75%, 80%, 85%, 90%, 95% (volume/volume) or more of MATRIGEL®. In some embodiments, the carrier comprises no more than about any one of 95%, 90%, 85%, 80%, 75%, 70%, 65%, 60%, 55%, or 50% of MATRIGEL®. In some embodiments, the composition comprises at least about any one of 100, 200, 500, 1000, 1200, 1300, 1400, 1500, 1600, 1700, 1800, 1900, 2000, 3000 or more μg/mL of fibrin. In some embodiments, the composition comprises no more than about any one of 3000, 2000, 1900, 1800, 1600, 1500, 1400, 1300, 1200, 1000, 500, 200, or 100 μg/mL of fibrin.

In some embodiments, the carrier is biodegradable. In some embodiments, the carrier is degraded in vivo in no more than about any one of 5 years, 4 years, 3 years, 2 years, 1 year, 6 months, 3 months, 1 month or less. In some embodiments, the carrier does not degrade for at least about any one of 2 months, 3 months, 6 months, 10 months, 1 year, 2 years, 3 years, 4 years, 5 years or more. In some embodiments, the engineered myogenic cells are intermixed with the carrier. In some embodiments, the engineered myogenic cells are dispersed in the carrier. In some embodiments, the engineered myogenic cells are embedded in the carrier. In some embodiments, the engineered myogenic cells are encapsulated by the carrier. In some embodiments, the engineered myogenic cells are not encapsulated by the carrier.

The composition comprises a suitable number of engineered myogenic cells. In some embodiments, the composition comprises a single engineered myogenic cell. In some embodiments, the composition comprises at least about any one of 10, 20, 50, 100, 200, 500, 1×10³, 2×10³, 5×10³, 1×10⁴, 2×10⁴, 5×10⁴, 1×10⁵, 5×10⁵, 1×10⁶, 1.5×10⁶, 2×10⁶, 3×10⁶, 4×10⁶, 5×10⁶, 6×10⁶, 7×10⁶, 8×10⁶, 9×10⁶, 1×10⁷, 5×10⁷ or more cells. In some embodiments, the composition comprises no more than about any one of 1×10⁷, 5×10⁶, 2×10⁶, 1×10⁶, 5×10⁵, 2×10⁵, 1×10⁵, 5×10⁴, 2×10⁴, 1×10⁴, 5×10³, 2×10³, 1×10³, 500, 200, 100, 50, 20, 10, or fewer cells. In some embodiments, the composition comprises about any one of 1-100, 10-1000, 100-1000, 1×10³-1×10⁴ 1×10⁴-1×10⁵, 1×10⁵-5×10⁵, 5×10⁵-1×10⁶, 1×10⁶-2×10⁶, 2×10⁶-3×10⁶, 3×10⁶-4×10⁶, 4×10⁶-5×10⁶, 5×10⁶-6×10⁶, 6×10⁶-7×10⁶, 7×10⁶-8×10⁶, 8×10⁶-1×10⁸, 1×10⁶-3×10⁶ 3×10⁶-5×10⁶, 5×10⁶-7×10⁶, 2×10⁶-2×10⁷, 5×10⁶-2×10⁷, or 1×10⁶-2×10⁷ cells. In some embodiments, the composition is a cell suspension comprising at least about 1×10⁶ myoblasts. In some embodiments, the composition is a muscle construct comprising myotubes and/or myofibers differentiated from at least about 1×10⁵ myoblasts.

In some embodiments, the composition is a pharmaceutical composition or formulation comprising a pharmaceutically acceptable carrier. As used herein, by “pharmaceutically acceptable” or “pharmacologically compatible” is meant a material that is not biologically or otherwise undesirable, e.g., the material may be incorporated into a pharmaceutical composition administered to an individual without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration. Techniques for formulation and administration of drugs may be found in “Remington's Pharmaceutical Sciences,” Mack Publishing Co., Easton, Pa., latest edition, which is incorporated herein by reference. The pharmaceutical compositions described herein may include other agents, excipients, or stabilizers to improve properties of the composition. The final form may be sterile and may also be able to pass readily through an injection device such as a hollow needle. The proper viscosity may be achieved and maintained by the proper choice of solvents or excipients. In some embodiments, the composition is suitable for administration to a human. In some embodiments, the composition is suitable for administration to a human by intramuscular or subcutaneous administration.

In some embodiments, there is provided a composition comprising engineered myogenic cells, a hydrogel carrier comprising extracellular matrix molecules (e.g., MATRIGEL®), and fibrin, wherein the engineered myogenic cells comprise muscle stem cells, myoblasts and/or differentiated cells thereof, and wherein the engineered myogenic cells are intermixed with the carrier.

In some embodiments, there is provided a composition comprising engineered myogenic cells, a hydrogel carrier comprising MATRIGEL® and fibrin, wherein the engineered myogenic cells comprise myoblasts, and wherein the engineered myogenic cells are intermixed with the carrier. In some embodiments, the myoblasts are Pax7⁻ Pax3⁺ MyoD⁺ myogenin⁻ or Pax7⁺ Pax3⁻ MyoD⁺ myogenin⁻ cells. In some embodiments, the myoblasts are produced from ESC or iPSC. In some embodiments, the myoblasts are rejuvenated myoblasts produced from adult myoblasts. In some embodiments, the myoblasts are produced by introducing into the adult myogenic cell a first nucleic acid encoding a LIN28A protein, a second nucleic acid encoding a PERT protein, and a p53 inhibitor (e.g., a third nucleic acid encoding a shRNA targeting p53). In some embodiments, the composition is a muscle construct.

In some embodiments, the engineered myogenic cells are embedded in the hydrogel carrier. In some embodiments, the engineered myogenic cells are homogenously dispersed within the hydrogel carrier. In some embodiments, the engineered myogenic cells are enwrapped by the hydrogel carrier.

In some embodiments, the weight ratio between the hydrogel carrier and the cells is at least about any one of 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1 or more. In some embodiments, the density of the engineered myogenic cells in the hydrogel carrier is at least about any one of 10, 50, 100, 500, 1000, 1×10³, 2×10³, 5×10³, 1×10⁴, 2×10⁴, 5×10⁴, 1×10⁵, 5×10⁵, 1×10⁶, 5×10⁷, 1×10⁷, 5×10⁸, 1×10⁸, 5×10⁹, 1×10⁹, 5×10¹⁰, 1×10¹⁰, 5×10¹¹, 1×10¹¹, 5×10¹¹, or 1×10¹² cells/mL.

In some embodiments, the hydrogel carrier has a viscosity of about 100-3000 mPa·s. In some embodiments, the hydrogel carrier has a viscosity of about any one of 100-500, 500-1000, 1000-1500, 1500-2000, 2000-2500, 2500-3000, 100-1000, 1000-3000, 1000-2000, or 500-2500 mPa·s. In some embodiments, the hydrogel carrier has a modulus of elasticity of about 50 kPa to about 2000 MPa. In some embodiments, the hydrogel carrier has a modulus of elasticity of at least about any one of 50 kPa, 100 kPa, 150 kPa, 200 kPa, 250 kPa, 300 kPa, 400 kPa, 500 kPa, 600 kPa, 700 kPa, 800 kPa, 900 kPa, 1 MPa, 5 MPa, 10 MPa, 50 MPa, 100 MPa, 500 MPa, 750 MPa, 1000 MPa, 1500 MPa or more. In some embodiments, the hydrogel carrier has a modulus of elasticity of about any one of 50 kPa-100 kPa, 100 kPa-200 kPa, 200 kPa-500 kPa, 500 kPa-1 MPa, 1 MPa-10 MPa, 10 MPa-100 MPa, 100 MPa-500 MPa, 500 MPa-1000 MPa, 1000 MPa-2000 MPa, 50 kPa-250 kPa, 100 kPa-1 MPa, 1 MPa-100 MPa, 100 MPa-2000 MPa, or 50 kPa-2000 MPa. In some embodiments, the hydrogel carrier is at least about any one of 60%, 65%, 80%, 85%, 90%, 95%, 98% or more hydrated. In some embodiments, the hydrogel carrier has a pH and salt concentrations that are suitable for the proliferation and/or differentiation of the engineered myogenic cells. In some embodiments, the hydrogel carrier has a pH between about 6.0 and 8.0.

### V. Kits and Articles of Manufacture

The present application further provides kits, formulations, unit dosages, and articles of manufacture for use in any one of the methods of delivery, methods of producing reprogrammed myogenic cells, and methods of treatment or diagnosis described herein.

In some embodiments, there is provided a kit useful for delivering an agent to an individual, comprising a composition comprising engineered myogenic cells that allow delivery of the agent, wherein the engineered myogenic cells comprise muscle stem cells, myoblasts and/or differentiated cells thereof, and a device for local administration of the composition to the individual. In some embodiments, the device is for intramuscular or subcutaneous administration. In some embodiments, the engineered myogenic cells comprise muscle stem cells, e.g., Pax7⁺ Pax3⁺ MyoD⁻ myogenin⁻, Pax7⁺ Pax3⁻ MyoD⁻ myogenin⁻, and/or Pax7⁻ Pax3⁺ MyoD⁻ myogenin⁻ cells. In some embodiments, the engineered myogenic cells comprise myoblasts, e.g., Pax7⁻ Pax3⁺ MyoD⁺ myogenin⁻ and/or Pax7⁺ Pax3⁻ MyoD⁺ myogenin⁻ cells. In some embodiments, the engineered myogenic cells comprise myoblasts produced from ESC or iPSC. In some embodiments, the engineered myogenic cells comprise reprogrammed myogenic cells produced from adult myogenic cells, e.g., by introducing into the adult myogenic cells a first nucleic acid encoding a LIN28A protein, a second nucleic acid encoding a TERT protein, and a p53 inhibitor such as a third nucleic acid encoding a shRNA targeting p53. In some embodiments, the engineered myogenic cells comprise transdifferentiated myogenic cells produced from adult somatic cells (e.g., fibroblasts). In some embodiments, the engineered myogenic cells comprise cells produced by differentiating muscle stem cells or myoblasts, such as myocytes, myotubes, and/or myofibers. In some embodiments, the composition is a muscle construct. In some embodiments, the composition is a muscle construct comprising engineered myogenic cells dispersed in a carrier, such as a carrier comprising ECM molecules and a cell adhesion molecule, e.g., MATRIGEL® and fibrin. In some embodiments, the kit is used for treating a disease or condition in an individual.

In some embodiments, there is provided a kit useful for producing reprogrammed myogenic cell from an adult myogenic cell, comprising: (a) an embryonic regulator; (b) a telomere-associated regulator; and (c) a cell cycle regulator. In some embodiments, the kit comprises one or more vector(s) comprising: (a) a first nucleic acid encoding LIN28A, (b) a second nucleic acid encoding TERT, and (c) a third nucleic acid encoding a p53 inhibitor. In some embodiments, the p53 inhibitor is a shRNA targeting p53. In some embodiments, the kit is used for treating a muscle disease or condition. In some embodiments, the kit is used for delivering an agent to an individual.

The kit may contain additional components, such as containers, reagents, culturing media, buffers, and the like to facilitate execution of any embodiment of the methods. For example, in some embodiments, the kit further comprises a cell collection and storage apparatus, which can be used to collect an individual's muscle stem cells and/or myogenic cells (e.g., myoblasts). In some embodiments, the kit further comprises culturing media or containers (e.g., petri dishes and plates) for proliferation and/or differentiation of muscle stem cells or myoblasts. In some embodiments, the kit further comprises immunostaining or histology reagents for assessing biomarkers of the engineered myogenic cells or engrafted myogenic cells.

The kits of the present application are in suitable packaging. Suitable packaging include, but is not limited to, vials, bottles, jars, flexible packaging (e.g., Mylar or plastic bags), and the like. Kits may optionally provide additional components such as interpretative information. The present application thus also provides articles of manufacture, which include vials (such as sealed vials), bottles, jars, flexible packaging, and the like.

The kits may also comprise instructions relating to the use of the engineered myogenic cells. In some embodiments, the kit further comprises an instructional manual, such as a manual describing a protocol according to any one of the methods of delivery, methods of producing reprogrammed myogenic cells, or methods of treatment or diagnosis described herein. The instructions may also include information on dosage, dosing schedule, and routes of administration of the engineered myogenic cells (including reprogrammed myogenic cells) using the kit for the intended treatment or diagnosis.

Also provided are unit dosage forms comprising the engineered myogenic cells (including reprogrammed myogenic cells) and formulations described herein. These unit dosage forms can be stored in a suitable packaging in single or multiple unit dosages and may also be further sterilized and sealed. In some embodiments, the composition (such as pharmaceutical composition) is contained in a single-use vial, such as a single-use sealed vial. In some embodiments, each single-use vial contains at least about 10⁶ cells. In some embodiments, each single-use vial contains enough engineered myogenic cells to be expanded to at least about 10⁶ cells. In some embodiments, the composition (such as pharmaceutical composition) is contained in a multi-use vial. In some embodiments, the composition (such as pharmaceutical composition) is contained in bulk in a container.

Those skilled in the art will recognize that several embodiments are possible within the scope and spirit of this invention. The invention will now be described in greater detail by reference to the following non-limiting exemplary embodiments and examples. The following exemplary embodiments and examples further illustrate the invention but, of course, should not be construed as in any way limiting its scope.

### EXEMPLARY EMBODIMENTS

The present application provides the following embodiments:
1. A method of delivering an agent to an individual in need thereof, comprising locally administering to the individual a composition comprising engineered myogenic cells, wherein the engineered myogenic cells comprise muscle stem cells, myoblasts and/or differentiated cells thereof, wherein the engineered myogenic cells engraft at the site of administration in the individual, and wherein the engineered myogenic cells are genetically modified to allow delivery of the agent to the individual.
2. The method of embodiment 1, wherein the composition is administered intramuscularly.
3. The method of embodiment 1, wherein the composition is administered subcutaneously.
4. The method of any one of embodiments 1-3, wherein the engineered myogenic cells comprise muscle stem cells.
5. The method of embodiment 4, wherein the muscle stem cells are Pax7⁺ Pax3⁺ MyoD⁻ myogenin⁻, Pax7⁺ Pax3⁻ MyoD⁻ myogenin⁻, and/or Pax7⁻ Pax3⁺ MyoD⁻ myogenin⁻ cells.
6. The method of any one of embodiments 1-5, wherein the engineered myogenic cells comprise myoblasts.
7. The method of embodiment 6, wherein the myoblasts are produced from embryonic stem cells (ESC) or induced pluripotent stem cells (iPSC).
8. The method of embodiment 6 or 7, wherein the myoblasts are Pax7⁻ Pax3⁺ MyoD⁺ myogenin⁻ and/or Pax7⁺ Pax3⁻ MyoD⁺ myogenin⁻ cells.
9. The method of any one of embodiments 1-8, wherein the engineered myogenic cells comprise myocytes produced from muscle stem cells or myoblasts.
10. The method of embodiment 10, wherein the myocytes are differentiated from myoblasts, wherein the myoblasts are produced from ESC or iPSC.
11. The method of embodiment 9 or 10, wherein the myocytes are Pax3⁻ Pax7⁻ MyoD⁺ myogenin⁺ cells.
12. The method of any one of embodiments 1-11, wherein the engineered myogenic cells are reprogrammed myogenic cells.
13. The method of embodiment 12, wherein the engineered myogenic cells are rejuvenated and/or de-differentiated myogenic cells produced from adult myogenic cells.
14. The method of embodiment 12, wherein the engineered myogenic cells are transdifferentiated myogenic cells produced from adult somatic cells.
15. The method of any one of embodiments 1-14, wherein the composition is a suspension of engineered myogenic cells.
16. The method of embodiment 15, wherein the composition is administered by injection.
17. The method of any one of embodiments 1-14, wherein the composition is a muscle construct comprising engineered myogenic cells.
18. The method of embodiment 17, wherein the composition is administered as a local implant.
19. The method of embodiment 17 or 18, wherein the muscle construct comprise myotubes.
20. The method of any one of embodiments 17-19, wherein the muscle construct comprise myofibers.
21. The method of any one of embodiments 17-20, wherein the muscle construct comprises PAX7⁺ myogenic cells.
22. The method of any one of embodiments 1-21, wherein the composition further comprises a carrier.
23. The method of embodiment 22, wherein the engineered myogenic cells are intermixed with the carrier.
24. The method of embodiment 22 or 23, wherein the carrier comprises extracellular matrix (ECM) molecules.
25. The method of embodiment 24, wherein the carrier comprises MATRTGEL®.
26. The method of any one of embodiments 22-25, wherein the carrier comprises a cell adhesion molecule.
27. The method of embodiment 26, wherein the cell adhesion molecule is fibrin.
28. The method of any one of embodiments 22-27, wherein the amount of the carrier in the composition is no more than about 95% (weight/weight).
29. The method of any one of embodiments 1-28, wherein the engineered myogenic cells are not produced from an immortalized cell line.
30. The method of any one of embodiments 1-29, wherein the engrafted engineered myogenic cells form a muscle tissue in the individual, wherein the muscle tissue allows delivery of the agent to the individual.
31. The method of embodiment 30, wherein the muscle tissue produces the agent.
32. The method of embodiment 31, wherein production of the agent is inducible.
33. The method of embodiment 31, wherein production of the agent is constitutive.
34. The method of any one of embodiments 30-33, wherein the muscle tissue allows local delivery of the agent to the individual.
35. The method of any one of embodiments 30-33, wherein the muscle tissue allows systemic delivery of the agent to the individual.
36. The method of any one of embodiments 30-35, wherein the muscle tissue recruits blood vessels from surrounding tissues in the individual.
37. The method of any one of embodiments 30-36, wherein the muscle tissue is fused to surrounding tissues in the individual.
38. The method of any one of embodiments 30-37, wherein the muscle tissue is removed from the individual after delivery of the agent to the individual for at least about 2 months.
39. The method of any one of embodiments 1-38, wherein the composition is administered once to the individual.
40. The method of any one of embodiments 1-39, wherein the engineered myogenic cells comprise a heterologous nucleic acid sequence encoding the agent.
41. The method of any one of embodiments 1-40, wherein the agent is delivered to the individual for at least about 2 months upon administration of the engineered myogenic cells.
42. The method of any one of embodiments 1-41, wherein the agent is selected from the group consisting of a metabolite, a nutrient, a small molecule drug, a biologic, a virus, an extracellular vesicle, a vaccine and a reporter.
43. The method of any one of embodiments 1-42, wherein the agent is secreted by the engineered myogenic cells.
44. The method of any one of embodiments 1-42, wherein the agent is expressed on the cell surface of the engineered myogenic cells.
45. The method of any one of embodiments 1-44, wherein the engineered myogenic cells allow delivery of a plurality of agents.
46. The method of any one of embodiments 1-45, wherein the composition comprises at least about 10⁵ of the engineered myogenic cells.
47. The method of any one of embodiments 1-46, wherein the engineered myogenic cells are autologous.
48. The method of any one of embodiments 1-46, wherein the engineered myogenic cells are allogeneic.
49. The method of any one of embodiments 1-48, wherein the engineered myogenic cells are non-immunogenic to the individual.
50. The method of any one of embodiments 1-49, wherein the engineered myogenic cells are non-tumorigenic.
51. The method of any one of embodiments 1-50, wherein the individual is a human individual.
52. The method of embodiment 51, wherein the individual is no more than about 3 years old.
53. The method of embodiment 51, wherein the individual is about 18 years old or older.
54. A method of treating or diagnosing a disease or condition in an individual, comprising delivering an agent to the individual using the method of any one of embodiments 1-53, wherein the agent treats or diagnoses the disease or condition.
55. The method of embodiment 54, wherein the disease or condition is a chronic disease or condition.
56. The method of embodiment 55, wherein the disease or condition is a metabolic disease.
57. The method of embodiment 56, wherein the disease or condition is diabetes, such as type I diabetes.
58. The method of embodiment 55, wherein the disease or condition is haemophilia, such as haemophilia B.
59. A method of preparing a non-human animal model of a disease or condition, comprising delivering an agent to a non-human animal using the method of any one of embodiments 1-53, wherein the agent is associated with the disease or condition.
60. The method of embodiment 59, wherein the agent is delivered to the bloodstream of the non-human animal.
61. The method of embodiment 59 or 60, wherein the agent is a human protein, RNA or metabolite.
62. The method of any one of embodiments 59-61, wherein the non-human animal is a rodent, such as a mouse or a rat.
63. A composition comprising engineered myogenic cells, a hydrogel carrier comprising extracellular matrix molecules, and fibrin, wherein the engineered myogenic cells comprise muscle stem cells, myoblasts and/or differentiated cells thereof, and wherein the engineered myogenic cells are intermixed with the carrier.
64. The composition of embodiment 63, wherein the carrier is MATRIGEL®.
65. The composition of embodiment 63 or 64, wherein the composition is a muscle construct.
66. The composition of embodiment 65, wherein the composition is a non-human meat product for consumption.
67. A kit for delivery of an agent to an individual, comprising a composition comprising engineered myogenic cells, wherein the engineered myogenic cells comprise muscle stem cells, myoblasts and/or differentiated cells thereof, and wherein the engineered myogenic cells are genetically modified to allow delivery of the agent; and a device for local administration of the composition to the individual.
68. Use of a composition comprising engineered myogenic cells in the preparation of a medicament for delivery of an agent to an individual in need thereof, wherein the medicament is formulated for local administration, wherein the engineered myogenic cells comprise muscle stem cells, myoblasts and/or differentiated cells thereof, wherein the engineered myogenic cells engraft at the site of administration in the individual, and wherein the engineered myogenic cells are genetically modified to allow delivery of the agent to the individual.
69. A method of producing a reprogrammed myogenic cell from an adult myogenic cell, comprising:
(a) introducing into the adult myogenic cell an embryonic regulator, a telomere-associated regulator, and a cell cycle regulator to provide a transduced myogenic cell; and
(b) culturing the transduced myogenic cell under conditions to obtain a reprogrammed myogenic cell.
70. The method of embodiment 69, wherein the reprogrammed myogenic cell is a muscle stem cell, myoblast, or myocyte.
71. The method of embodiment 69 or 70, wherein the reprogrammed myogenic cell is rejuvenated.
72. The method of any one of embodiments 69-71, wherein the adult myogenic cells express high levels of p21^{WAF1}, p16^{INK4a}, and let-7 microRNAs.
73. The method of any one of embodiments 69-72, wherein the adult myogenic cell can undergo no more than about 30 population doublings.
74. The method of any one of embodiments 69-73, wherein the reprogrammed myogenic cell can undergo at least about 90 population doublings.
75. The method of any one of embodiments 69-74, wherein the reprogrammed myogenic cell is de-differentiated.
76. The method of any one of embodiments 69-75, wherein the reprogrammed myogenic cell is PAX3⁺.
77. The method of any one of embodiments 69-76, wherein the embryonic regulator is LIN28A.
78. The method of any one of embodiments 69-77, wherein the telomere-associated regulator is TERT.
79. The method of any one of embodiments 69-78, wherein the cell cycle regulator is a p53 inhibitor.
80. The method of embodiment 79, wherein the p53 inhibitor is a short hairpin RNA (shRNA) targeting p53.
81. The method of any one of embodiments 1-76, wherein step (a) comprises introducing into the adult myogenic cell a first nucleic acid encoding a LIN28A protein, a second nucleic acid encoding a TERT protein, and a third nucleic acid encoding a shRNA targeting p53.
82. The method of embodiment 81, the first nucleic acid, the second nucleic acid and/or the third nucleic acid are present in one or more vectors.
83. The method of embodiment 82, wherein the one or more vectors are viral vectors.
84. The method of any one of embodiments 69-83, wherein the adult myogenic cell is a human cell.
85. The method of any one of embodiments 69-84, wherein the transduced myogenic cell is cultured in a medium comprising Dulbecco's Modified Eagle Medium (DMEM) with about 20% fetal bovine serum (FBS).
86. A reprogrammed myogenic cell prepared by the method of any one of embodiments 69-85.
87. A method of delivering an agent to an individual in need thereof, comprising locally administering to the individual a composition comprising an effective amount of the reprogrammed myogenic cells of embodiment 86 or differentiated cells thereof, wherein the reprogrammed myogenic cell or differentiated cells thereof are genetically modified to allow delivery of the agent to the individual.
88. A method of treating a muscle disease or condition in an individual in need thereof, comprising administering to the individual an effective amount of reprogrammed myogenic cells of embodiment 86 or differentiated cells thereof.
89. The method of embodiment 87 or 88, wherein the adult myogenic cell is obtained from the individual.
90. The method of embodiment 88 or 89, wherein the disease or condition is sarcopenia or cachexia.
91. A kit for producing reprogrammed myogenic cells, comprising: (a) a first nucleic acid encoding a LIN28A protein, (b) a second nucleic acid encoding a TERT protein, and (c) a p53 inhibitor.
92. A method of treating a non-human animal model of a disease or condition, comprising delivering an agent to the non-human animal using the method of any one of embodiments 1-53, wherein the agent treats the disease or condition.

### EXAMPLES

The examples below are intended to be purely exemplary of the present application and should therefore not be considered to limit the invention in any way. The following examples and detailed description are offered by way of illustration and not by way of limitation.

### Example 1: Long-Term, Safe Engraftment of Engineered Myogenic Cells and Constructs

This example demonstrates long-term engraftment of engineered myogenic (referred herein as “iMusc”) cells and constructs thereof by intramuscular or subcutaneous transplantation in mice. The iMusc myoblast or myocyte suspensions were injected intramuscularly into the muscles of mice. iMusc constructs prepared using iMusc myoblasts could be transplanted under the skin of mice. A hemispheroid iMusc construct comprising iMusc myoblasts encapsulated in a MATRIGEL®-fibrin matrix could be transplanted intramuscularly. Inventors discovered that the iMusc cells and constructs were able to differentiate into myofibers that persisted at the site of transplantation for more than one year, and recruit vasculature from the surrounding murine muscle tissue. Additionally, the iMusc tissues were able to express transgenes (such as GFP), did not impede the functions of surrounding tissues and organs in the body, and did not develop into tumors over time. The iMusc cells can be engineered to carry out a variety of functions, such as to metabolize metabolites (e.g., fatty acids, glucose, fructose, alcohol, etc.), and to secrete nutrients and biologics. These properties make iMusc cells and constructs useful cell carriers to deliver agents either locally to surrounding tissues in the host, or systemically by secretion into the circulation.

### Methods

Maintenance and Differentiation of iMusc Cells

Myoblasts were prepared from human embryonic stem cells (ESC) or induced pluripotent stem cells (iPSC) according to known protocols in the art. See, for example, e.g., Darabi, 2008; Borchin, 2013; Shelton, 2016. To maintain the myoblasts (referred herein as “iMusc myoblasts”) as myoblasts, the cells were maintained in proliferation media (PM; DMEM, 20% FBS, 1% Pen-Strep) and passaged before 80% confluency each time. To differentiate the iMusc myoblasts into myocytes, iMusc myoblasts were allowed to reach 100% confluency and cultured in differentiation medium (DM; DMEM/F12, 2% horse serum, 1% Pen-Strep, 1% L-glutamine) for 2 days. For further differentiation into myotubes and myofibers, iMusc myocytes were maintained in DM for another 11 days.

### Intramuscular Transplantation of Cells

NOD-SCID gamma mice were anaesthetized with a mixture of 70 mg/kg of body weight ketamine and 7 mg/kg of body weight xylazine by intraperitoneal (IP) injection. Anesthesia was monitored by pinching the toe. 3 million iMusc myoblasts were resuspended in 20 μL MATRIGEL® according to the manufacturer's dilution factor, then directly injected into the tibialis anterior muscle using an insulin syringe (BD). Alternatively, 3 million iMusc myoblasts were resuspended in a master mix of 7.92 μL undiluted MATRIGEL®, and 0.08 μL aprotinin (Sigma, 80 μg/mL) and 2 μL thrombin (Sigma, 10 U/mL) dissolved in DM. The resuspended cells were then seeded on a petri dish, creating a hemispheroid droplet. 0.8 μL of fibrinogen was added and mixed within the hemispheroid. The hemispheroid was allowed to solidify at 37° C. via the hanging drop method (1 hr). The solidified hemispheroid was then transplanted into the tibialis anterior muscle.

Preparation of iMusc Constructs

All iMusc constructs were engineered in individual wells of a 6-well plate. Each well was coated with 2 g of polydimethylsiloxane (PDMS) gel. To make 2 g of PDMS gel, 1.8 g of type 184 silicone elastomer base and 0.2 g of curing agent (Dow Chemical Corporation) were mixed evenly to ensure a homogenous mix. Next, the PDMS gel was subjected to a vacuum for 30 min to remove bubbles created during the mixing. The gel was poured gently into the 6-well plate, and further vacuumed until no bubbles were observed. The plate was cured overnight in a 37° C. incubator. 6 mm-long surgical silk sutures were pinned horizontally, 12 mm apart, using stainless steel minutien pins in the center of each dish. The construct was sterilized with 70% ethanol (30 min), washed 3 times with PBS, and further sterilized with UV radiation (30 min). The PBS was aspirated and the construct dried by placing in the incubator (1 hr). A master mix of 396 μL of DM, 100 μL thrombin (Sigma, 10 U/mL) and 4 μL of aprotinin (Sigma, 80 μg/mL) was added into the well. The well was ensured to be evenly coated and sutures positioned correctly, before 200 μL of fibrinogen (Sigma, 20 mg/mL) was evenly added. The plate was shaken gently and the fibrin gel was polymerized at room temperature (10 min). The fibrin gel was further polymerized in an incubator (1 hr). 400,000 cells in 2 mL DM were seeded on top of the fibrin gel surface. The plate was shaken gently to distribute the cells evenly throughout the well. The DM media was changed every 3 days for 2 weeks, before the iMusc construct was used for subcutaneous transplantation under the dorsal skin of NOD-SCID gamma mice.

### Results

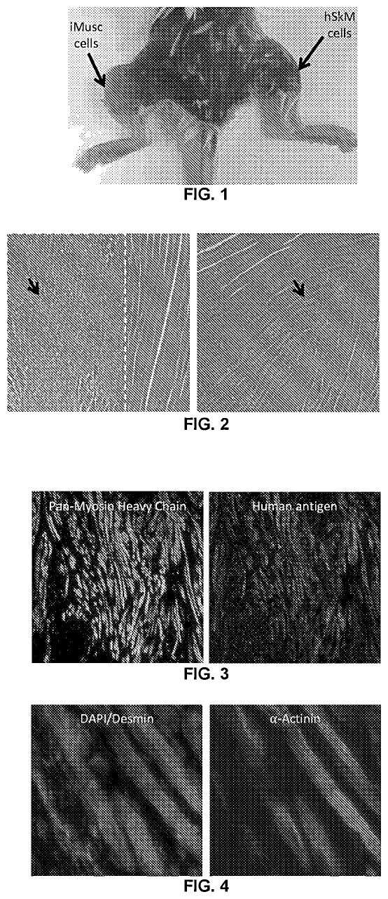

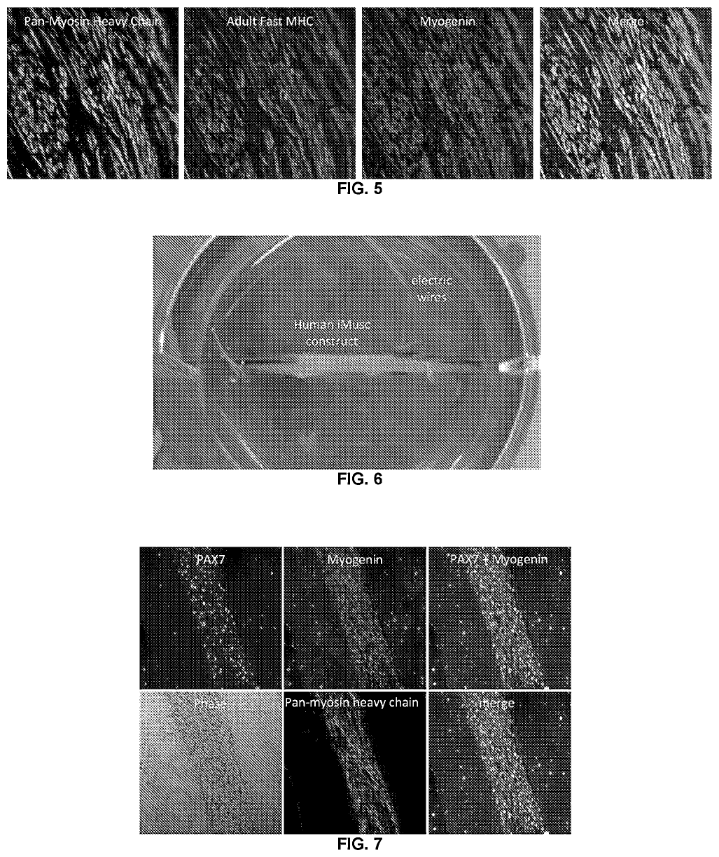

We first discovered that human iMusc myoblasts or myocytes could be engrafted by intramuscular injection of cell suspensions in diluted MATRIGEL® into the tibialis anterior muscles of NOD-Scid gamma mice (FIG. 1). As shown in FIG. 2, one year after orthotopic transplantation, the human iMusc cells were detected via hematoxylin and eosin (H&E) histology at the injection site. In contrast, few human skeletal muscle cells were detected at a similar intramuscular injection site in the mouse one-year post transplantation. As shown in FIGS. 3-5, the iMusc myoblast grafts robustly differentiated into myosin heavy chain (MHC)⁺, desmin⁺, α-actinin⁺, myogenin⁺ myofibers one year after transplantation.

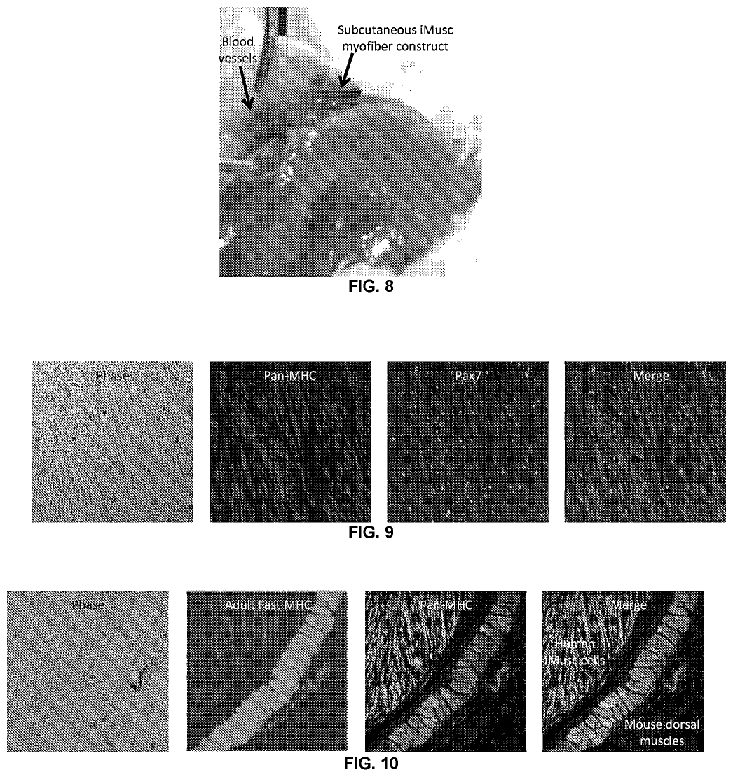

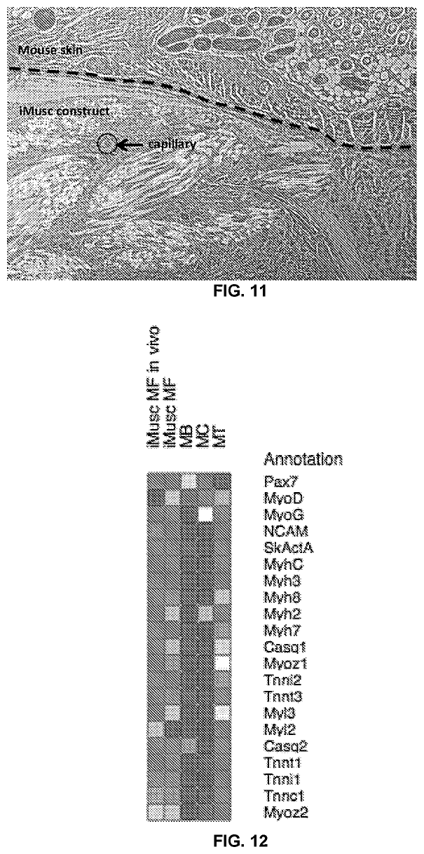

In a second experiment, we prepared a human iMusc construct in vitro with iMusc myoblasts (FIG. 6). Immunostaining results showed both PAX7⁺ iMusc cells and myogenin/pan-MHC⁺ iMusc-myotubes in the iMusc construct (FIG. 7). The iMusc construct was implanted subcutaneously under the dorsal skin of a NOD-Scid gamma mouse (FIG. 8). As shown in FIGS. 9-10, one year after the implantation, human PAX7⁺ iMusc cells, myogenin/pan-MHC⁺ iMusc-myotubes and adult fast MHC⁺ iMusc-myotubes persisted in the subcutaneous environment. Surprisingly, as shown in FIG. 11, the iMusc tissue was able to recruit capillaries from the surrounding murine muscle tissue. Quantitative RT-PCR results in FIG. 12 demonstrate that iMusc myofiber tissue from the mouse has similar myogenesis gene expression profile as iMusc myofiber construct in vitro. Compared to myogenic cells (e.g., myoblasts, myocytes and myotubes) from primary human skeletal muscle, the iMusc myofiber tissues express higher levels of nearly every myogenic marker. For example, compared to myotubes from primary human skeletal muscle, the iMusc myofiber tissues express higher levels of PAX7, MYH8, MYH7, CASQ1, MYOZ1, TNNI2, and MYL3

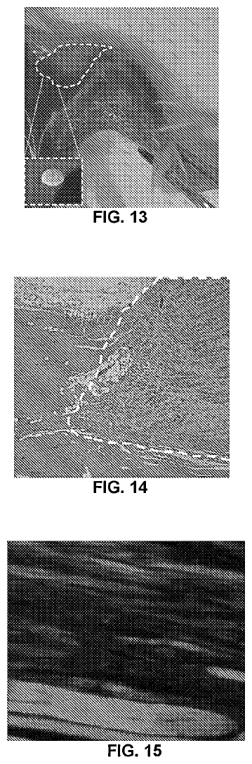

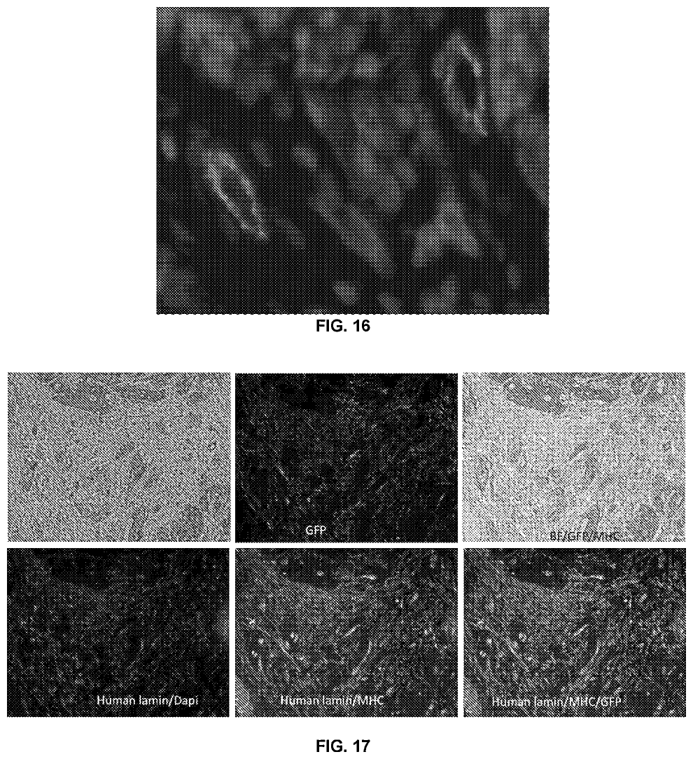

In a third experiment, we prepared a hemispheroidiMusc construct having iMusc myoblasts encapsulated in a MATRIGEL®-fibrin matrix, and implanted the hemispheroid into the quadriceps muscles of a NOD-Scid gamma mouse (FIG. 13). As shown by H&E histology, the iMusc implant persisted one year after implantation (FIG. 14). The iMusc myoblasts differentiated into α-actinin⁺striated myofibers (FIG. 15), and recruited vascular capillaries (FIG. 16).

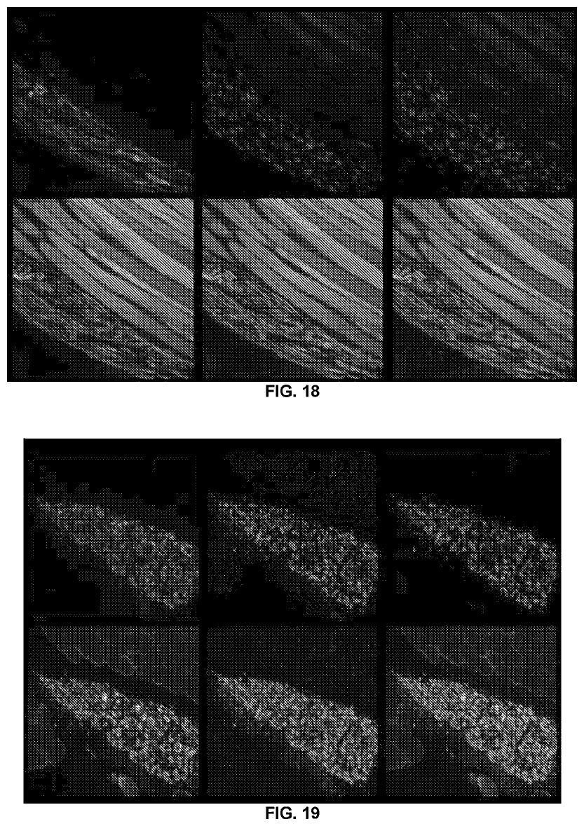

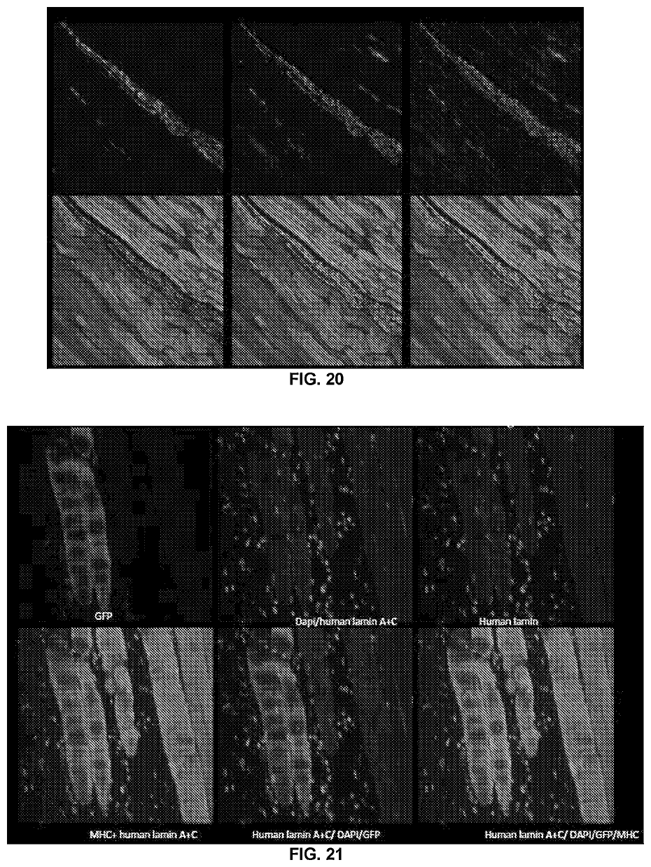

In a fourth set of experiments, we injected iMusc myocytes encoding a GFP transgene into uninjured tibialis anterior muscles of NOD-Scid gamma mice. One year after transplantation, the iMusc myocytes differentiated into myosin heavy chain (MHC)⁺ myofibers that expressed GFP (FIG. 17). Similarly, iMusc myoblasts encoding a GFP transgene were injected into uninjured (FIG. 18) or cardiotoxin-injured (FIGS. 19-20) tibialis anterior muscles of NOD-Scid gamma mice. The iMusc myoblasts differentiated into myosin heavy chain (MHC)⁺ myofibers that expressed GFP (FIGS. 17-20). As shown in FIG. 21, the GFP⁺ human lamin A/C+ iMusc cellswere fused with mouse myofibers one year after transplantation into cryoinjured tibialis anterior muscles of NOD-Scid gamma mice.

Notably, the iMusc cells and constructs stayed at the location of transplantation one year following transplantation. This is in sharp contrast to red blood cell-based delivery systems, in which the cell carriers circulate in the entire body in order to deliver biologic agents. The iMusc cells and constructs gave rise to muscle tissues that recruited vasculature from the surrounding mouse tissues, which could allow secretion of agents into the circulation to allow systemic delivery. Yet, at one-year post transplantation, no human antigen was found outside the site of transplantation in the mice. Also, histopathology reports revealed no malignancies in the mice at one-year post transplantation, which suggests that the iMusc cells and constructs are safe candidates as long-term delivery vehicles, unlike cell-line based systems that have heightened risk of tumorigenicity.

We have demonstrated fully controllable growth and differentiation of iMusc cells and constructs in vitro and upon local administration in vivo. The iMusc grafts persisted over a long period of time and were able to express transgenes in the intramuscular and subcutaneous environments. The iMusc grafts also did not impede the normal functions of surrounding tissues and organs in the body. We are engineering iMusc cells with biologic secretion functions. Such iMusc cells and constructs can be transplanted intramuscularly or subcutaneously into individuals to provide long-term local or systemic delivery of useful agents, such as biologic drugs, etc.

### Example 2. Use of iMusc to Treat Genetic Diseases

The therapeutic efficacies of engineered iMusc cells and constructs for systemic delivery of recombinant proteins can be shown in mouse models of genetic diseases.

### Modified Insulin (Insm) for Type 1 Diabetes Mellitus (T1D)

### Construction & Validation

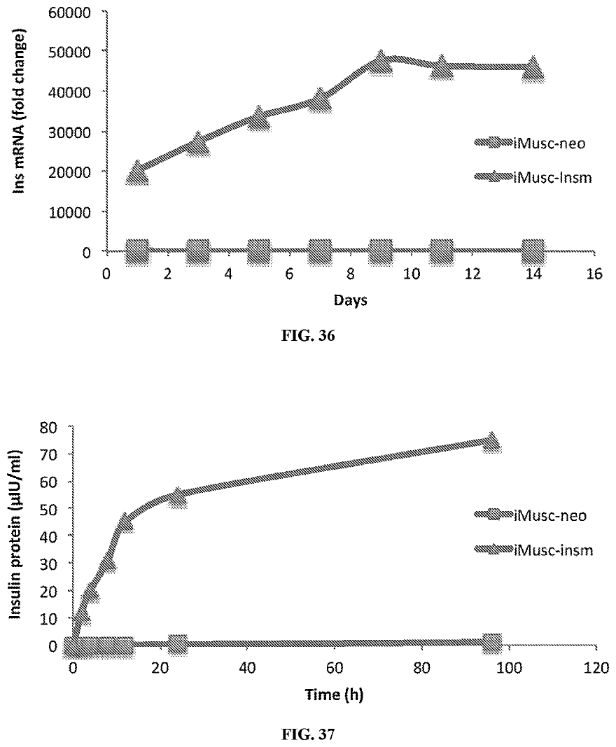

The iMusc myoblasts were stably transduced via retrovirus, with the CMV promoter linked to human proinsulin cDNA containing furin endoprotease sites instead of prohormone convertase sites (“iMusc-Insm”; Groskreutz et al., 1994). To test if differentiated iMusc cells synthesized and processed the proinsulin, insulin expression was determined by qRT-PCR and ELISA (Mercodia), after the iMusc-neo (control iMusc cells without transgene) and iMusc-Insm cells were allowed to differentiate for 14 days in the presence of 1% KSR, and iMusc insulin mRNA was measured after every 2-3 days of differentiation (FIG. 36). To measure the levels of secretion of bioactive insulin, over 14 days of differentiation, 1×10⁶ iMusc-neo and iMusc-Insm cells were cultured overnight in serum-free medium supplemented with 20 mM glucose, washed, and then incubated in the same medium containing 0.5% BSA and aprotinin (0.1 mg/ml). Aliquots were taken from the culture medium every few hours for 4 days, and insulin levels were measured by ELISA (Mercodia). High levels of insulin protein were secreted (FIG. 37).

### In Vitro Function

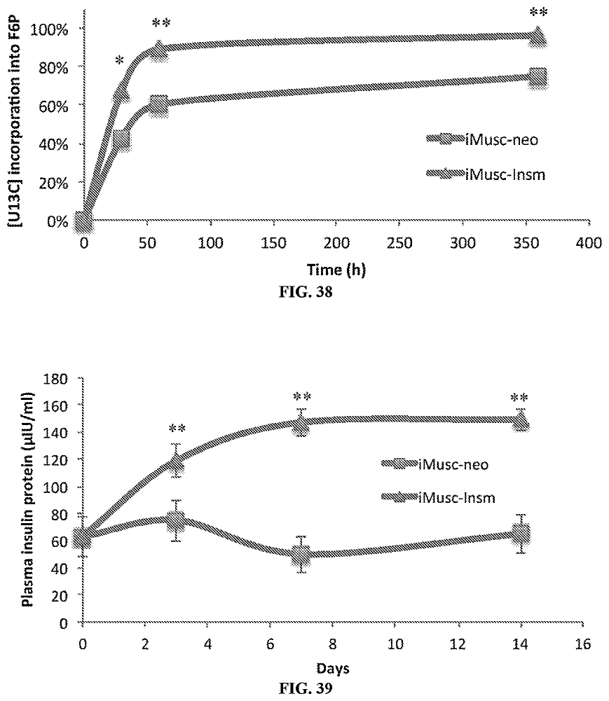

To determine whether the secreted insulin could increase glucose metabolism, we collected 48 hour-conditioned media from iMusc-neo and iMusc-Insm cells. After 14 days of differentiation, non-transduced hSkM (human skeleton muscle) cells were cultured overnight in serum-free medium supplemented with 5 mM glucose, washed, and then incubated in 48 hour-conditioned media from iMusc-neo and iMusc-Insm cells containing 5 mM [U-¹³C]glucose. After 30 min, 60 min and 6 hours of exposure, the cells were washed, harvested for extracts, and sent for LC-MS/MS analysis (Waters) to test for ¹³C-incorporation rates into the intracellular glycolytic intermediate fructose-6-phosphate. We found a higher flux of glycolysis after exposure to iMusc-Insm conditioned media (P<0.01), indicating that the higher level of insulin in iMusc-Insm conditioned media was stimulating glucose metabolism in hSkM cells (FIG. 38).

Transplantation of iMusc-Insm Cells into Diabetic Nude Mice

To determine whether differentiated iMusc-Insm cells were able to produce bioactive insulin and treat diabetes mellitus in vivo, 5×10⁶ iMusc-Insm and iMusc-neo myoblast cells were transplanted by direct skeletal muscle injection into 6-8 week old male athymic nude mice (B6.Cg-Foxn1ⁿⁿ/J; Jackson Labs) that have received daily intraperitoneal injections of 40 mg/kg streptozotocin (Tocris Bioscience), dissolved in 0.1M citrate buffer (pH 4.5), for 5 consecutive days, to model diabetes mellitus. Glycemia and insulinemia were measured in fed mice every 3-7 days, for 3 weeks after injection of the cells. Blood glucose was measured using a glucometer (Roche), while plasma insulin was measured by ELISA (Mercodia).

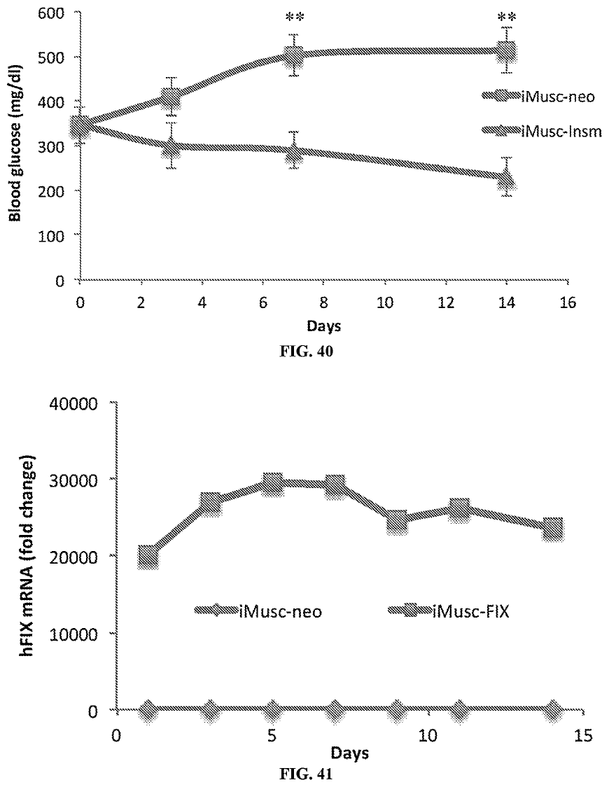

Our results showed that after streptozotocin treatment, fed insulin levels dropped from 131±16 μIU/ml to ˜63 μIU/ml. After transplantation of iMusc-neo cells, the fed insulin levels remained low at 50-75 μIU/ml (FIG. 39). However, after transplantation of iMusc-Insm cells, the fed insulin levels were steadily restored to ˜149 μIU/ml (P<0.01) by day 14 (FIG. 39). Conversely, after streptozotocin treatment, fed glucose levels increased from ˜110 mg/dl to ˜346 mg/dl. After transplantation of iMusc-neo cells, the fed glucose levels continued increasing to >500 mg/dl (FIG. 40). However, after transplantation of iMusc-Insm cells, the fed glucose levels steadily decreased to ˜229 mg/dl (P<0.01) by day 14 (FIG. 40).

### Factor IX (FIX) for Hemophilia B

### Construction & Validation

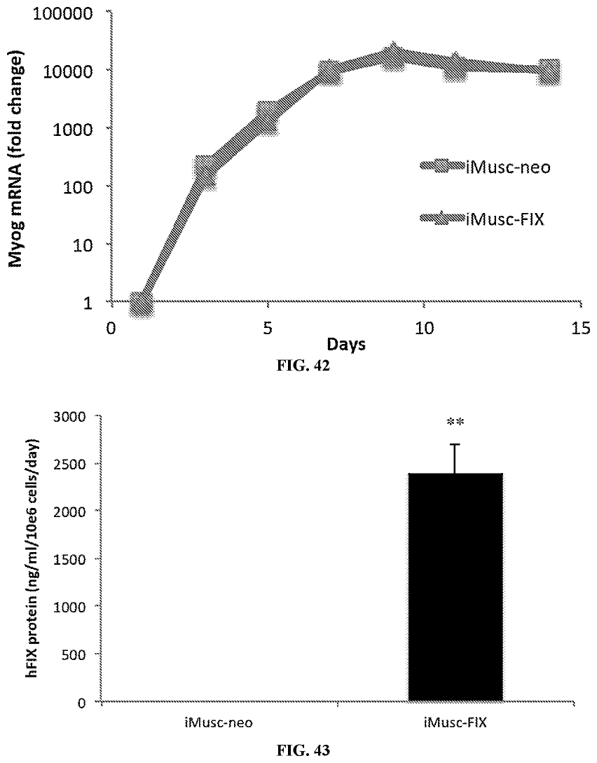

The iMusc myoblasts were stably transduced via retrovirus, with the CMV promoter linked to human FIX cDNA (“iMusc-FIX”). To test if differentiated iMusc cells synthesized and processed the human Factor IX (hFIX), expression was determined by qRT-PCR and ELISA (Abcam), after the iMusc-neo and iMusc-FIX cells were allowed to differentiate for 14 days in the presence of 1% KSR, and iMusc hFIXmRNA was measured after every 2-3 days of differentiation (FIG. 41). The expression of the myogenin (Myog) gene was used as a marker of differentiation (FIG. 42). To measure the levels of secretion of human Factor IX, after 14 days of differentiation, 1×10⁶ iMusc-neo and iMusc-FIX cells were cultured overnight in serum-free medium supplemented with 20 mM glucose, washed, and then incubated in the same medium containing 0.5% BSA and aprotinin (0.1 mg/ml). Aliquots were taken from the culture medium after 24 hours, and human Factor IX levels were measured by ELISA (Abcam). High levels of FIX protein were secreted (P<0.01, FIG. 43).

Transplantation of iMusc-FIX Cells into Hemophilic Nude Mice

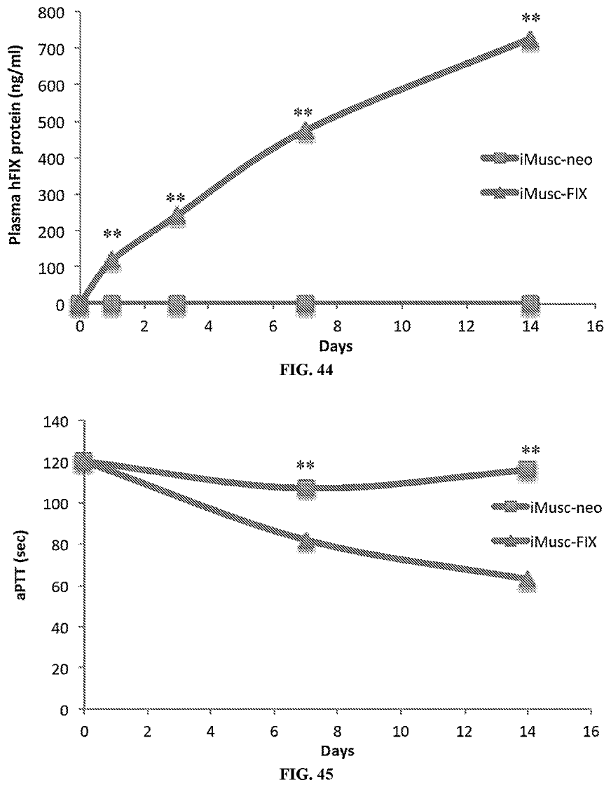

To facilitate in vivo testing of the iMusc-FIX cells, mice with hemophilia B (B6.129P2-F9^{tm1Dws/J}; Jackson Labs) were crossed to athymic nude mice (B6.Cg-Foxn1^{nu}/J; Jackson Labs) to obtain F9^{-/Y}; Foxn1^{nu/nu} mice in 2 generations. To determine whether differentiated iMusc-FIX cells were able to produce bioactive Factor IX in vivo and treat hemophilia B, 30×10⁶ iMusc-FIX and iMusc-neo cells were transplanted by direct skeletal muscle injection into 6-8 week old F9^{-/Y}; Foxn1^{nu/nu} mice. Plasma human Factor IX levels were measured by ELISA (Abcam). After transplantation of iMusc-neo cells, the hFIX levels remained near zero (FIG. 44). However, after transplantation of iMusc-FIX cells, the hFIX levels steadily increased to ˜726 ng/ml (P<0.01) by day 14 (FIG. 44). The activated partial thromboplastin time (aPTT) was measured with a Fibrintimer COA system every week for 2 weeks after injection of the cells. After transplantation of iMusc-neo cells, the aPTT remained high at 107-120 sec (FIG. 45). However, after transplantation of iMusc-FIX cells, the aPTT steadily decreased to ˜63 sec (P<0.01) by day 14 (FIG. 45).

### Example 3: Reprogrammed Myogenic Cells

During ageing, adult muscle stem cells' regenerative properties decline, as they enter a senescent state and lose both their proliferative and differentiation capacities. In contrast, embryonic and fetal muscle progenitors possess heightened proliferative capacities despite many rounds of mitosis, and manifest a more robust regenerative response upon injury and transplantation. How embryonic and fetal progenitors delay senescence and maintain their proliferative and differentiation capacities despite multiple rounds of mitosis, remains unknown. It is also unclear if defined embryonic factors can rejuvenate adult muscle progenitors to confer extended proliferative and differentiation capacities, without reprogramming their lineage or cell fates. Here we report that only a minimal combination of LIN28A, hTERT, and sh-p53 (LTS), all of which are tightly regulated factors during fetal development, is needed to prevent senescence in adult muscle progenitors. LTS muscle progenitors showed an extended proliferative capacity, maintained a normal karyotype, underwent myogenesis normally in late-passages, and did not manifest tumorigenesis nor aberrant lineage differentiation. LTS treatment rescued the pro-senescence phenotype of aged cachexia patients' muscle progenitors, and promoted their engraftment for skeletal muscle regeneration in vivo. When we examined the mechanistic basis for LIN28A's role in the LTS combo, we found that let-7 microRNAs could not fully explain how LIN28A promoted muscle progenitor self-renewal. Instead, LIN28A was mildly promoting oxidative phosphorylation (OxPhos) in adult muscle progenitors to optimize mitochondrial reactive oxygen species (mtROS) and mitohormetic signaling. Optimized mtROS induced a variety of stress responses and glycolysis, thereby promoting adult muscle progenitor self-renewal. Perturbation of mtROS levels specifically abrogated LIN28A-driven HIF1A and glycolysis, and thus LTS progenitor self-renewal, without affecting normal or TS progenitors. Our findings connect embryonic factors to mitohormesis in the context of adult progenitor rejuvenation, with implications for ageing-related muscle degeneration in cachexia and sarcopenia.

### Methods

### Cell Culture and Differentiation

Young adult primary human skeletal muscle (HSKM) progenitors isolated from a 20-year old female subject's quadriceps muscles (Gibco) were seeded onto plates coated with 0.1% gelatin solution (Merck-Millipore) and incubated at 37° C., 5% CO₂ with growth medium, comprising of DMEM/F-12 (Gibco) with 20% fetal bovine serum (FBS) (GE Healthcare), 1% L-glutamine (Gibco) and 1% penicillin-streptomycin (Gibco). At each passage, after reaching 80% confluence, cells were trypsinized and diluted 1:4. Differentiation was initiated by replacing growth media with differentiation medium, comprising of DMEM/F-12, 2% KnockOut Serum Replacement (Gibco), 1% L-glutamine (Gibco) and 1% penicillin-streptomycin (Gibco), when the young adult HSKM progenitors were 80-100% confluent. These young adult HSKM progenitors were previously validated to be 100% MYOD⁺ during the proliferative stage, with robust expression of myogenic markers after differentiation (Chua et al., 2019).

### Cachexia Patient Cell Lines

Aged primary human skeletal muscle progenitors were derived from the rectus abdominus of two aged patient donors with cachexia (age/weight: 80 years/55 kg and 84 years/44 kg) during tumor-resection surgery. Procedures were performed in accordance to ethical legislation and Institutional Review Board guidelines. Muscle progenitors were isolated according to previously published protocols (Skuk et al., 2010). Cells were maintained in culture medium and differentiated with differentiation medium as described above.

### Virus Production

GP2-293 cells (Clontech) were seeded at 10% confluency and transfected with a 12 μL: 3.33 μg: 0.66 μg mix of PEI (1 mg/mL): retroviral plasmids (Addgene #1773, #26357): VSV-G envelope plasmid (Addgene #8454). 293FT FMK cells (Clontech) were seeded at 10% confluency and transfected with a 42 μL: 7 μg: 6.3 μg: 0.7 μg mix of PEI (1 mg/mL): lentiviral plasmid (Addgene #19119): dR8.2 packaging plasmid (Addgene #8455): VSV-G envelope plasmid (Addgene #8454). 293FT and GP2 cells were initially cultured in DMEM (Gibco) with 10% FBS (GE Healthcare), 1% L-glutamine (Gibco) and 1% penicillin-streptomycin (Gibco). 24-hour-post transfection, growth medium was replaced with DMEM (Gibco) with 20% FBS (GE), 1% L-glutamine (Gibco) and 1% penicillin-streptomycin (Gibco). Viral supernatants were collected within the 48-hour to 96-hour window and filtered with a 0.45 μm filter (Sartorius).

### Virus Transduction and Selection

HSKM cells or patient derived-myoblasts were seeded in 6-well plates (Falcon) in growth medium comprising of DMEM-F12 (Gibco) with 20% FBS (GE), 1% L-glutamine (Gibco), 1% penicillin-streptomycin (Gibco). Cells were then transduced with concentrated viral supernatant (polybrene) and incubated for 16-24 hours. Transduced cells were selected with growth media containing either hygromycin, puromycin, or G418 (all antibiotics are from InvivoGen).

### Population Doubling Curve

1.5×10⁴ cells were seeded in one gelatin-coated well of a 6-well plate (Falcon) with growth medium comprising of DMEM/F-12 (Gibco) with 20% fetal bovine serum (FBS) (GE), 1% L-glutamine (Gibco) and 1% penicillin-streptomycin (Gibco). Upon reaching a confluency of 80-100%, cells were lifted with 0.25% trypsin (Gibco) and counted, 1.5×10⁴ cells were then subcultured. This process was repeated until cells could no longer achieve 80% confluency, or until a period of 100 days. Recorded cell counts were calculated as cumulative population doubling levels and plotted over the number of days in culture.

### Quantitative PCR

RNA was extracted by TRIzol (Thermo Fisher) and reverse transcribed with Superscript III (Thermo Fisher) according to manufacturer's instructions. The resulting cDNA was diluted 5× before performing qPCR with KAPA SYBR FAST on ABI Prism 7900HT (Applied Biosystems) according to manufacturers' instructions.

### Western Blots

Protein was extracted with RIPA buffer (Thermo Fisher) supplemented with protease inhibitor cocktails I and II (Sigma) and phosphatase inhibitor cocktail set III (Calbiochem). Protein was quantified with Pierce BCA protein assay kit (Thermo Fisher) and analyzed with a Sunrise Tecan plate reader. After SDS-PAGE andelectro-transfer onto PVDF membranes (GE Healthcare), Western blotting was performed with the following primary antibodies and concentrations: GAPDH (sc-365062; Santa Cruz; 1:10000), PAX3 (AB_528426; DSHB; 0.5 μg/mL), Twist2 (ab66031; Abcam; 1 μg/mL), IMP1/2/3 (sc-271785; Santa Cruz; 1:1000), HMGA2 (#5269S; Cell Signaling; 1:1000), tubulin (ab210797; Abcam; 1:1000), p53 (sc-126; Santa Cruz; 1:100), citrate synthase (G-3) (sc-390693; Santa Cruz; 1:1000). Blots were stained with secondary antibodies conjugated with HRP, 1:2500, (Promega W401B, W402B) and visualized with ECL prime western blotting detection reagent kit (GE Healthcare).

### Immunofluorescence

Cells were first washed with PBS (Thermo Fisher) and fixed with 4% PFA (MS). Cells were stained with the following primary antibodies and concentrations, MHC-IIb eFluor 660 (50-6503-32; Thermo Fisher; 1:100), α-actinin (sc-7453; Santa Cruz; 1:500), myogenin (sc-576; Santa Cruz; 1:200), Oxo-guanine-8 (ab206461; Abcam; 1:400). The following secondary antibodies were also used together with non-conjugated primary antibodies, Goat-anti-mouse Alexa Fluor 488 (A11001; Thermo Fisher; 1:500), Goat-anti-rabbit Alexa Fluor 594 (A11012; Thermo Fisher; 1:500), Goat-anti-mouse Alexa Fluor 647 (A21235; Thermo Fisher; 1:500). DAPI (d9542; Sigma) was used as a nuclear counter stain according to manufacturer's recommendations. Stained cells were imaged with a Zeiss fluorescence microscope.

### MiRNA Quantitative PCR

500 ng of total RNA extracted by TRIzol (Thermo Fisher) was amplified by miScript RT Kit (Qiagen). With the resulting cDNA libraries, real-time quantitative PCR was performed using the miScript SYBR Green PCR kit (Qiagen) on ABI Prism 7900HT (Applied Biosystems) according to manufacturers' instructions. The following miScript Primer Assays (Qiagen) were used in RT-qPCR, Hs_let-7a_2 (MS00031220), Hs_let-7b_1 (MS00003122), Hs_let-7e_3 (MS00031227), Hs_let-7g_2 (MS00008337), Hs_RNU6-2_11 (MS00033740) and Hs_SNORD61_11 (MS00033705).

### Illumina Transcriptomics and Analysis

TRIzol (Thermo Fisher) was used to extract total RNA and purified by ethanol precipitation. cRNA libraries were generated with Illumina TotalPrep RNA Amplification Kit (Thermo Fisher) and hybridized to HumanHT-12 v4.0 BeadChips (Illumina) according to manufacturer's protocol. Beadchips were scanned with a Bead Array Reader (Illumina) and results were extracted with Illumina BeadScan and Genome Studio. Further analysis was performed with Gene Set Enrichment Analysis (GSEA) (Broad Institute). Principal components analysis of the dataset was calculated in R version 3.1.2 using the prcomp function of the stats package.

### Extracellular Flux and Oxygen Consumption Measurements

Cells were seeded onto Seahorse XFe96 cell culture plate (Agilent) at a density of 6000-8000 cells/well with culture medium for 24 hours. Subsequently, media was changed to Seahorse minimal media and incubated in a non-CO₂, 37° C. incubator for one hour as per manufacturer's instructions. Utility plate was loaded with the following nutrient/drugs, Port A—10 mM glucose (for glycolysis) or 10 mM pyruvate (for OxPhos); Port B—1 μM oligomycin; Port C—0.5 μM FCCP; Port D—0.5 μM Antimycin A. Assay was run on a Seahorse XF extracellular flux analyzer (Agilent) according to manufacturer's protocols.

### Senescence-Associated β-Galactosidase Assays

Senescence-associated β-galactosidase (SA-β-gal) activity was determined with the Senescence Cells Histochemical Staining Kit (Sigma-Aldrich) according to manufacturer's protocols. Six representative images were captured on a TS100 inverted light microscope (Nikon) and camera module. For each cell line, cells stained blue by cleaved X-gal, indicating the presence of SA-β-gal, were counted as a percentage of the total number of cells in all images.

### Cytogenetics

Cells were seeded in gelatin-coated six-well plates and cultured up to a maximum of 50% confluency to avoid myoblast fusion. Cells were first treated with colcemid and BrdU overnight before harvesting with EDTA. A fixative solution (1:3=glacial acetic acid:methanol) was used to fix pelleted cells prior to slide preparation, Giemsa banding and mounting. Twenty metaphase spreads were prepared for each cell line for detailed analyses and karyotyping.

let-7 microRNA Mimic

HSKM, TS and LTS cells were seeded in gelatin-coated six-well plates at a density of 4×10⁴ cells/well and maintained with culture medium. PEI (14.5 ng/μL), mature Mirvana hsa-let-7a-5p (#4464066; Assay ID: MC10050; Thermo Fisher) and/or hsa-let-7b-5p (#4464066; Assay ID: MC11050; Thermo Fisher) mimic (0.25 μM) or Cy5-conjugated scramble RNAi control, was mixed with serum-free DMEM to a total volume of 200 μL. This mixture was transfected onto each well.

### KC7F2, ROS Modulators and 2-Deoxyglucose (2-DG)

HSKM, TS and LTS cells were seeded in gelatin-coated six-well plates at a density of 4×10⁴ cells/well and maintained with culture medium. One day after seeding, culture medium was replaced with culture medium containing either paraquat (Sigma-Aldrich; 2 μM), hydrogen peroxide (ICM Pharma; 100 μM), DTT (Thermo Fisher; 250 μM), 2-deoxyglucose (Sigma-Aldrich; 2.5 mM), KC7F2 (Sigma-Aldrich; 10 μM) or DMSO/H₂O controls. For ROS modulators, cells were allowed to grow for 72 hours post-treatment, while for 2-DG, cells were allowed to grow for eight days post-treatment.

Flow Cytometry Oxidation State Assay with Mito-Grx1-roGFP2

Retroviruses were created with the pLPCX mito Grx1-roGFP2 plasmid according to the virus production subsection. HSKM, TS and LTS cells were transduced with the roGFP2 retrovirus for 48 hours. Cells were allowed to expand to 15-cm tissue culture plates and then harvested for fluorescence-activated cell sorting for roGFP2+ cells using the FITC channel with a BD FACS Aria II cell sorter. HSKM, TS and LTS cells were seeded in gelatin-coated six-well plates at a density of 4×10⁴ cells/well and maintained with culture medium. One day after seeding, culture medium was replaced with culture medium containing either paraquat (Sigma-Aldrich; 2 μM), hydrogen peroxide (ICM Pharma; 100 μM), or DTT (Thermo Fisher; 250 μM). Cells were allowed to grow for 48 hours and then harvested for flow cytometry using a BD LSR Fortessa x-20 analyzer. HSKM, TS and LTS cells were used as non-fluorescent gating controls for each of their respective roGFP2+ counterparts. For each cell line, FSC, SSC, 405 nm/AmCyan and 488 nm/FITC channels were captured. FCS files were exported from FACS Diva and imported into FlowJo, where raw-value CSV files were exported. Raw values of 405 nm/488 nm ratio was calculated and then averaged for each cell line and condition. The % oxidized value was calculated according previously published methods (Hanson et al., 2004), where the difference between the 405/488 value of the vehicle control and DTT-treated condition was divided by the difference between the 405/488 value of the hydrogen peroxide and DTT-treated conditions.

### Intramuscular Injection

Four-month-old NSG ice were anaesthetized with a mixture of ketamine and xylazine (70 mg/kg & 7 mg/kg respectively) via intraperitoneal injection. Successful anesthetization was determined by a lack of response to toe pinching. Muscular cryoinjury on the tibialis anterior (TA) was performed before injection of cells and in vehicle controls. This involved the application of a dry-ice-chilled 4-mm metal probe onto exposed TA muscle for three cycles of ten seconds. 300,000 HSKM or cLTS cells were resuspended into 20 μL hESC-qualified MATRIGEL® Matrix (BD) diluted according to manufacturer's recommendation. Using an insulin syringe (BD), the cell and matrix suspension was then injected into the TA muscle. Analgesia in the form of meloxicam (5 mg/kg/24 hours) was administered subcutaneously for 48 hours post-operation.

### Results

LTS Factors Prevent Progenitor Senescence while Permitting Normal Differentiation

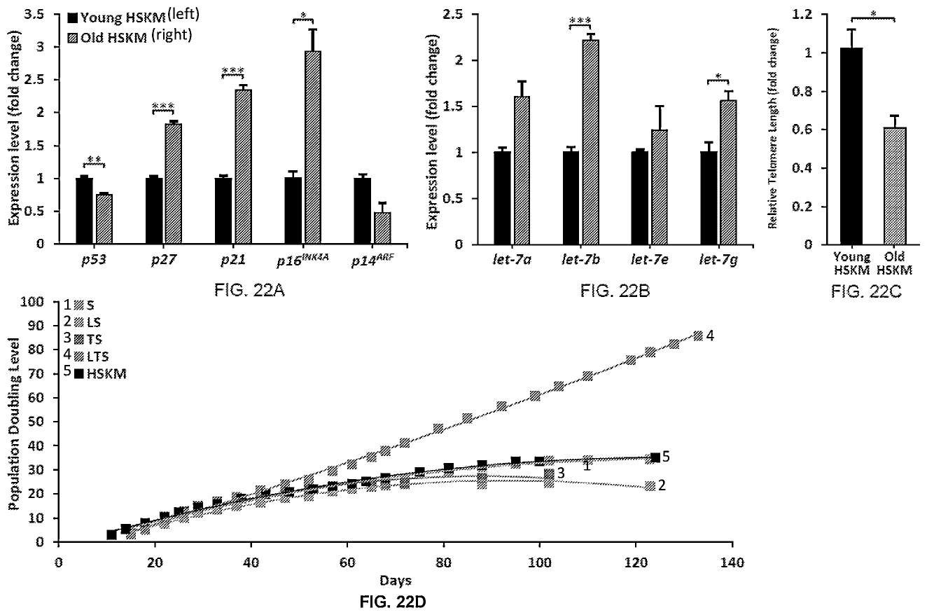

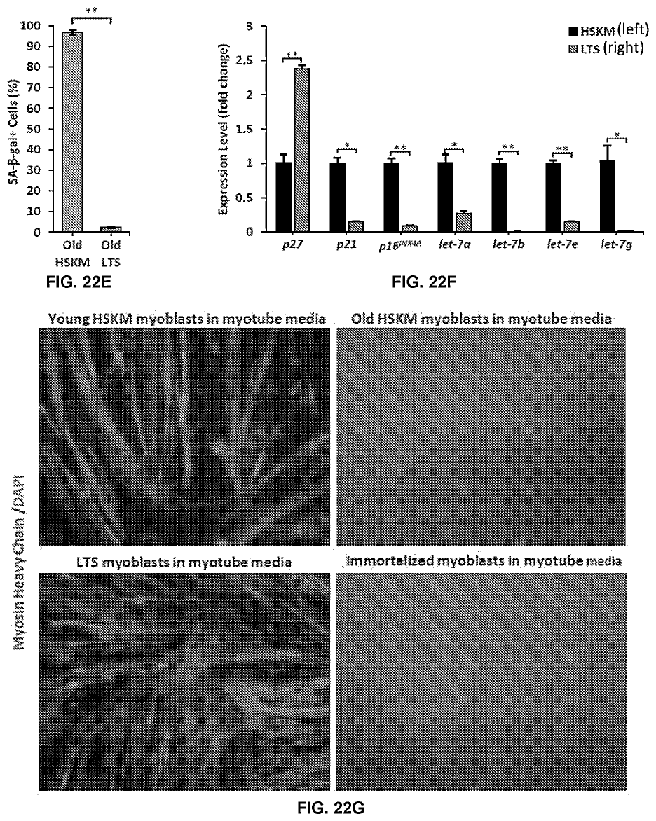

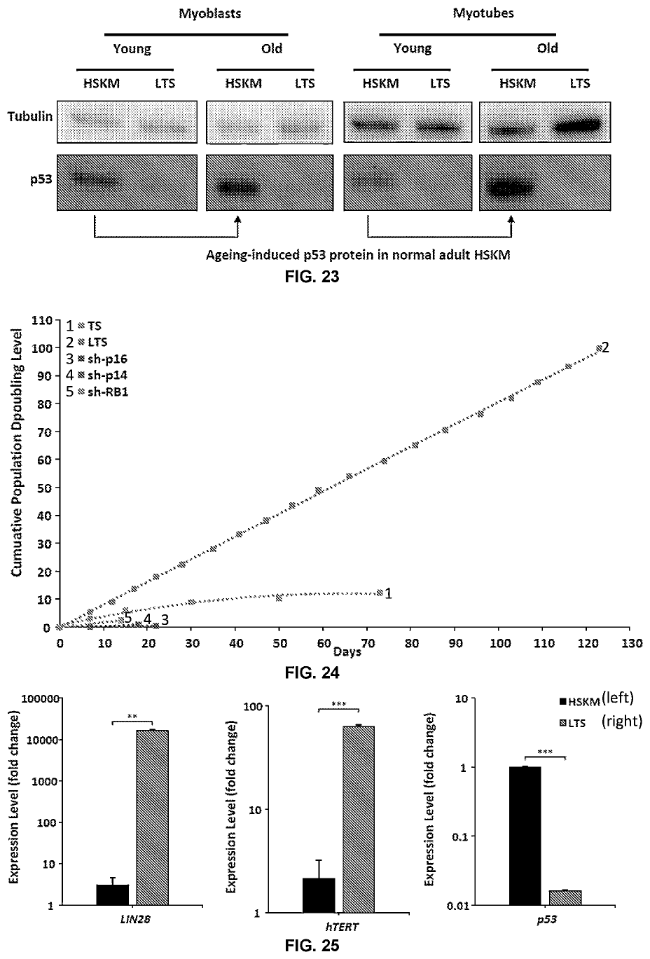

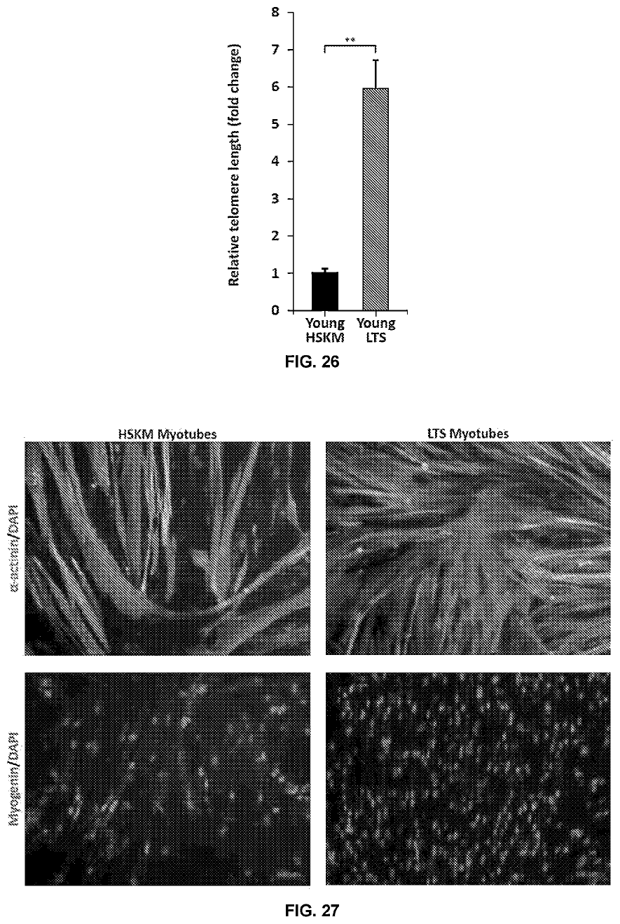

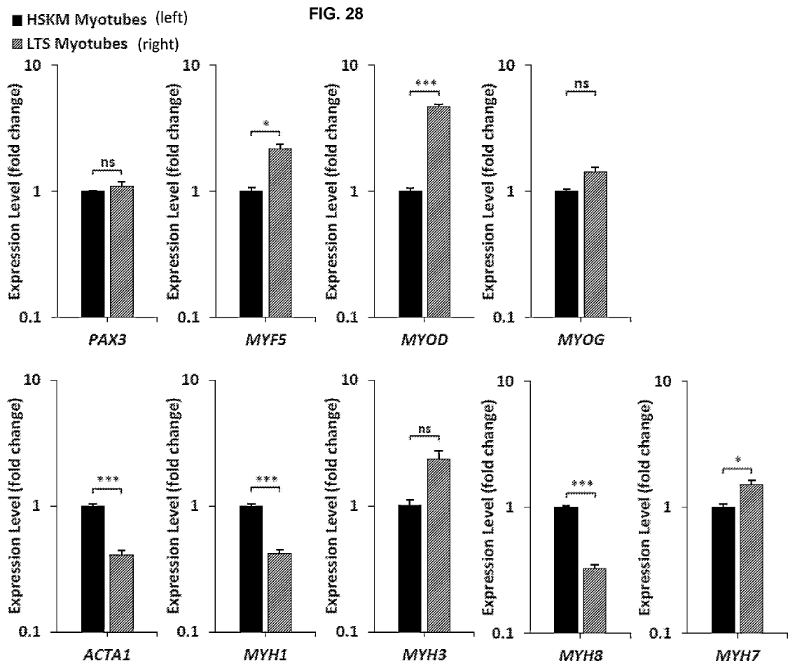

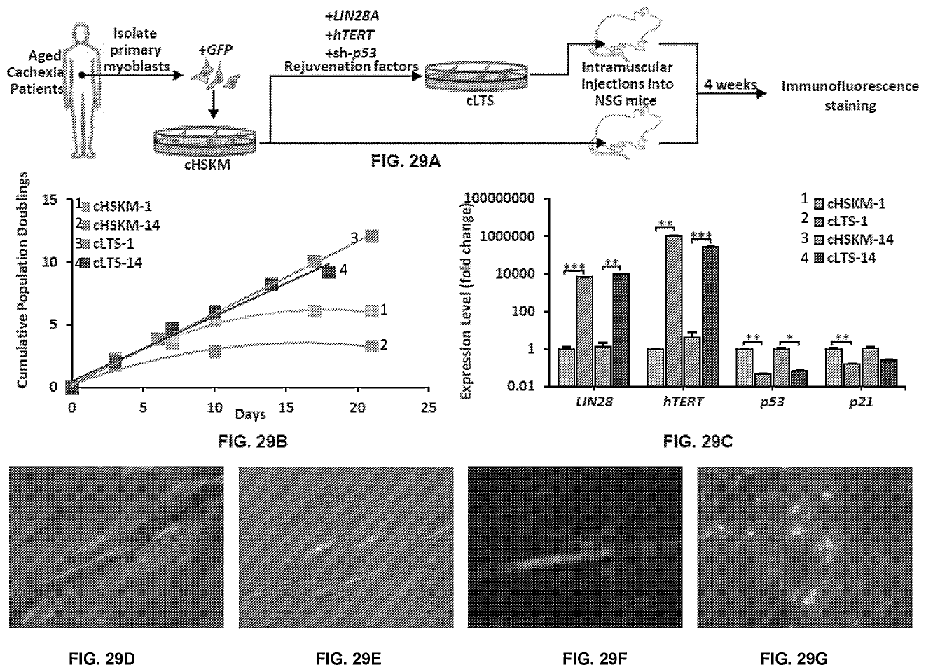

To address this question, we used young and aged primary adult human skeletal muscle (HSKM) progenitors to screen for a variety of embryonically-regulated factors that are not lineage-specific. We define young progenitors as less than 5 population doublings, and old progenitors as more than 20 population doublings. First, we found that old adult HSKM progenitors, compared to young adult HSKM progenitors, showed significantly higher levels of cell cycle inhibitors such as p21^{WAF1}, p27^{KIP1}, and p16^{INK4a} (FIG. 22A). Furthermore the anti-proliferative family of let-7 microRNAs, especially let-7b/g, also accumulated to higher levels in old adult HSKM progenitors, compared to young adult HSKM progenitors (FIG. 22B), consistent with studies of human muscle samples (Drummond et al., 2011). In contrast, the telomeres of old HSKM progenitors were significantly shorter than young HSKM progenitors (FIG. 22C). Although the mRNA expression levels of p53 and p14^{ARF} trended towards downregulation, we found out later that p53 protein was accumulating to higher levels in old muscle progenitors (FIG. 23). These preliminary results spurred us to conduct a mini-screen of embryonic factors to attempt to prevent or reverse the pro-senescence trend. We screened for a variety of embryonically regulated factors, including LIN28A to regulate the accumulating let-7 miRNAs and other developmental targets, hTERT to lengthen and restore the telomeres, and short hairpin RNAs (shRNAs) against p53 (which also transactivates p21^{WAF1}), p16^{INK4a}, p16^{ARF} or Rb to repress the cell cycle arrest inducers (Beauséjour et al., 2003; Christy et al., 2015; Coletti et al., 2002; Kudlow et al., 2008; Pajcini et al., 2010; Schwarzkopf et al., 2006) and mimic the fetal growth phases. In contrast, we omitted screening any mutant oncogenes which overcome existing senescence, instead of prevent senescence, and which might cause transformation or aberrant differentiation of progenitors instead (Chua et al., 2019). Our results showed that young adult primary HSKM progenitors eventually underwent senescence, plateauing sigmoidally at ˜30 population doublings, and no single factor alone could prevent the senescence (FIGS. 22D and 24). However, when LIN28A was combined with hTERT and sh-p53 (LTS; FIG. 25), human muscle progenitors could self-renew and proliferate indefinitely, beyond 90 population doublings, with a linear population doubling curve throughout. Furthermore, each factor was necessary in the LTS combo, as lacking any one of the three factors resulted in either senescence (FIG. 22D) or apoptosis. When 100 day-old adult HSKM progenitors and 100 day-old LTS progenitors were examined for senescence-associated β-galactosidase (SA-β-gal) staining, 96.7% of wildtype HSKM progenitors were SA-β-gal⁺, whereas only 2.2% of LTS progenitors were SA-β-gal⁺, indicating that the LTS combo was profoundly protective against senescence (FIG. 22E). When examined in detail, we found that the LTS combo (did prevent the ageing-induced accumulation of p21^{WAF1}, p16^{INK4a}, and let-7 microRNAs (FIG. 22F), while lengthening the telomeres (FIG. 26).

We then tested if the LTS progenitors could still differentiate properly. We expect transformed progenitors to be unable to differentiate properly, since progenitors need to completely withdraw from the cell cycle before they can undergo terminal differentiation. Surprisingly, we found that 100-day-old LTS progenitors could still robustly differentiate into myosin heavy chain (MHC)⁺, α-actinin⁺, myogenin⁺ multinucleated myotubes, just like young HSKM progenitors (FIGS. 22G and 27).

In contrast, immortalized HSKM progenitors harboring oncogenic CDK4^{R24C}, cyclin D1 and hTERT failed to differentiate properly, and only expressed very weak levels of MHC, with few multinucleate myotubes (FIG. 22G). Similarly, 100-day-old senescent HSKM progenitors could not differentiate into multinucleated myotubes (FIG. 22G). We also tested if LTS progenitors were reprogrammed into primitive mesenchymal or mesodermal progenitors by subjecting them to adipogenic, osteogenic, and chondrogenic differentiation conditions. We did not detect significant adipogenesis, osteogenesis or chondrogenesis. We also profiled LTS cells for their mRNA expression of myogenic markers. LTS myotubes were only mildly weaker in some myogenic markers (ACTA1, MYH1, MYH8), but also mildly stronger in several other myogenic markers (MYF5, MYOD, MYH7), while maintaining similar expression in some myogenic markers (MYOG, MYH3) compared to young adult HSKM myotubes (FIG. 28). These results indicate that LTS progenitors can undergo normal differentiation.

### LTS Factors can Rejuvenate Aged Muscle Progenitors for Transplantation

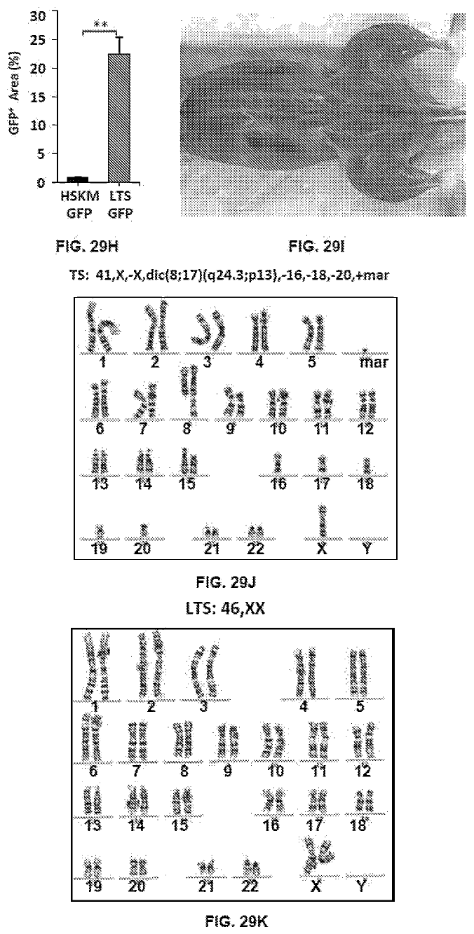

To test the utility of the LTS factors in diseased muscle progenitors, we transduced muscle progenitors from aged cancer cachexia patients. We previously found that cancer cachexia can lead to suppression of myocyte growth and myocyte apoptosis (Fukawa et al., 2016). When we cultured aged cachexia patients' HSKM progenitors, we also found that they rapidly underwent senescence within 6 population doublings (FIGS. 29A-29B), which is significantly less than healthy young adult muscle progenitors (˜30 population doublings, FIG. 22D). However, upon treatment with the LTS factors (FIG. 29B), the cachectic HSKM progenitors successfully averted rapid senescence with linear population doubling curves (FIG. 29A). To test if LTS treatment can produce engraftable progenitors for cell therapy, LTS cachectic progenitors were massively expanded, whereas control HSKM progenitors were pooled together. Equal numbers of GFP⁺ LTS and control progenitors were injected orthotopically into the tibialis anterior muscles of NOD-scid gamma (NSG) mice after cryoinjury. Immunofluorescence staining for GFP showed that there was significant engraftment of LTS cachectic progenitors 4 weeks after injection (FIGS. 29D-29G), whereas little engraftment of GFP⁺ control progenitors was observed in the regenerating muscles. The GFP⁺ LTS human progenitors had mostly differentiated into elongated myofibers (FIG. 29D), or fused with mouse myofibers to form GFP⁺ domains within the myofibers (FIGS. 29E-29F), whilst a minority persisted as self-renewing human progenitors on the periphery of the myofibers (FIG. 29G). Overall, LTS treatment dramatically increased the engraftment efficiency of human muscle progenitors (FIG. 29H), which is typically very low (Gussoni et al., 1997; Mendell, 1995). Thus, even aged muscle progenitors' regenerative functions can be restored with LTS treatment.

We were also interested to test if LTS progenitors would lead to tumorigenesis. However, no tumors were observed in NSG mice (FIG. 29I), the most rigorous model for testing tumorigenesis (Quintana et al., 2008), even after 12 months. When we subjected late-passage LTS progenitors to chromosomal analysis, the cells still displayed a normal karyotype, whereas late-passage TS progenitors displayed various aneuploid karyotypes, including the loss of numerous chromosomes, and the appearance of dicentric, ring and marker chromosomes (FIGS. 29J-29K). These results suggested that LTS may promote chromosomal stability despite lower levels of p53. Thus, the LTS factors did not transform any progenitors into cancer cells in vitro or in vivo.

LTS Myoblasts are Less Aged and Less Differentiated than Primary Myoblasts

We compared LTS to primary HSKM cells, both myoblasts and myotubes, and both young and old, using transcriptomic profiling to assess their cellular states. Principal components analysis (PCA) revealed that the different cell-types were clearly separated by ageing and differentiation status (FIG. 30A). The differentiation vectors, defined between HSKM myoblasts and myotubes, was approximately parallel to the LTS cells' differentiation vector, indicating that myogenesis led to similar changes in multidimensional RNA expression in both cell-types. The senescence vector defined by HSKM young, intermediate, and old myoblasts, was approximately orthogonal to the differentiation vector. Based on the axes defined by the differentiation and senescence vectors, LTS progenitors appear less aged and less differentiated compared to young adult HSKM progenitors (FIG. 30A).

To confirm this observation, we performed qRT-PCR for a variety of myogenic markers to assess the differentiation status of young adult HSKM and LTS progenitors. Our results showed that LTS progenitors were significantly higher in expression of PAX3, an embryonic myogenic transcription factor, than HSKM progenitors (FIG. 30C). While the myoblast commitment factor MYOD was higher (FIG. 30C), the myogenic determination factor MYF5 was lower (FIG. 30C), and the myogenic differentiation factor myogenin MYOG remained similarly low (FIG. 30C). In contrast, most other myogenic differentiation markers were even lower in LTS progenitors than HSKM progenitors, including skeletal muscle actin Al ACTA1 (FIG. 30C), and several myosin heavy chain isoforms (FIG. 30C).

We also performed Western blots for PAX3 and TWIST2 protein expression in LTS, TS and HSKM cells. LTS progenitors showed the highest PAX3 protein levels, compared to HSKM and TS progenitors (FIG. 30B). All the progenitors showed significant declines in PAX3 and TWIST2 protein levels, as they differentiated into myotubes (FIG. 30B), and as expected of myogenesis (Liu et al., 2017; Quintana et al., 2008). The increased PAX3 protein, an embryonic myogenic transcription factor, supports the transcriptomic observations on LTS progenitors.

LIN28A Aids Rejuvenation Via let-7-Independent Effects on mRNA Expression

To dissect the mechanism for LIN28A's rejuvenating effect, we returned to primary human progenitors cultured ex vivo. Our RNA profiling studies earlier had shown that let-7 family members accumulated to higher levels in ageing progenitors (FIG. 22B). Both TS and LTS transduction lowered the let-7 levels, with LIN28A further suppressing let-7b, let-7e, and let-7g levels (FIG. 31A). To test if LIN28A's rejuvenation effect in muscle progenitors was mediated by let-7, we transfected mature let-7 mimics in an attempt to abrogate transgenic LIN28A's effect on proliferation (FIG. 31A). However, we found that neither let-7a, nor let-7b, nor both, had any effect on LTS progenitors' proliferation rate (FIG. 31B). Moreover, let-7 overexpression also failed to increase the numbers of senescent SA-β-gal⁺ cells (FIG. 31C), even though important let-7 targets such as IMP1/2/3 and HMGA2 (Shyh-Chang and Daley, 2013) had been almost completely depleted by the let-7 overexpression (FIG. 31D). These results suggest that let-7 repression alone cannot fully explain LIN28A's effect on muscle progenitor self-renewal.

Thus, we re-analyzed the transcriptomic profiles of the LTS progenitors, and compared them against TS progenitors by GSEA. Curiously, the top downregulated signatures were almost completely dominated by interferon response targets (FIG. 31E). This suggests that TS progenitors were undergoing inflammaging-induced senescence, which LTS helped to prevent, consistent with findings that the interferon response is activated during senescence (De Cecco et al., 2019). Our analysis revealed that LIN28A's effect was primarily metabolic in nature, with the top upregulated signatures consisting of hypoxia targets, glycolysis genes and mitochondrial oxidative phosphorylation (OxPhos) genes, and also stress response signatures such as the unfolded protein response (UPR) mediated by XBP1S, and the DNA damage repair (DDR) response mediated by p53 (FIG. 31E).

LIN28A Optimizes OxPhos and mtROS to Induce HIF1A-Glycolysis

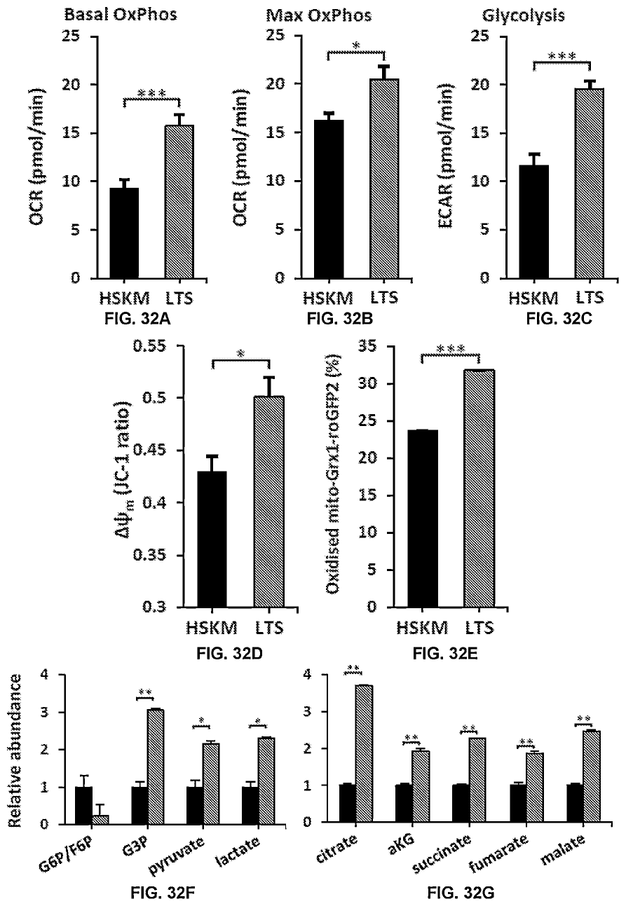

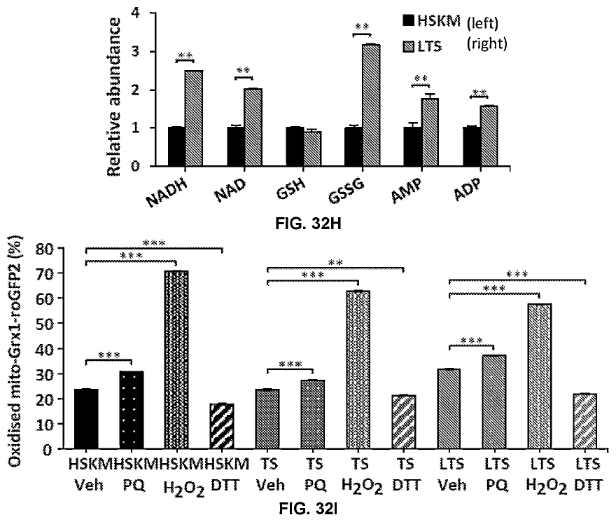

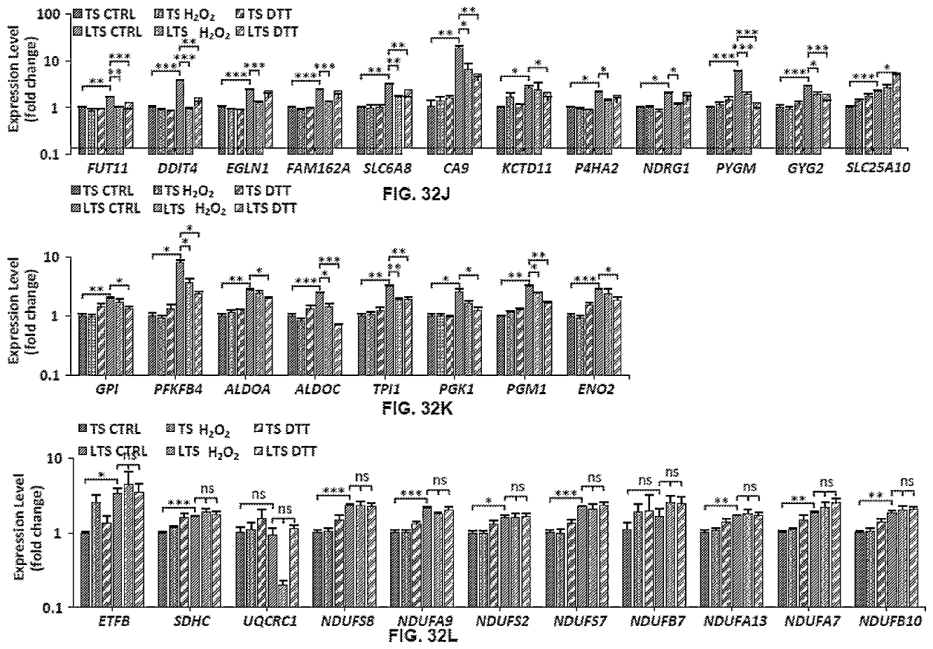

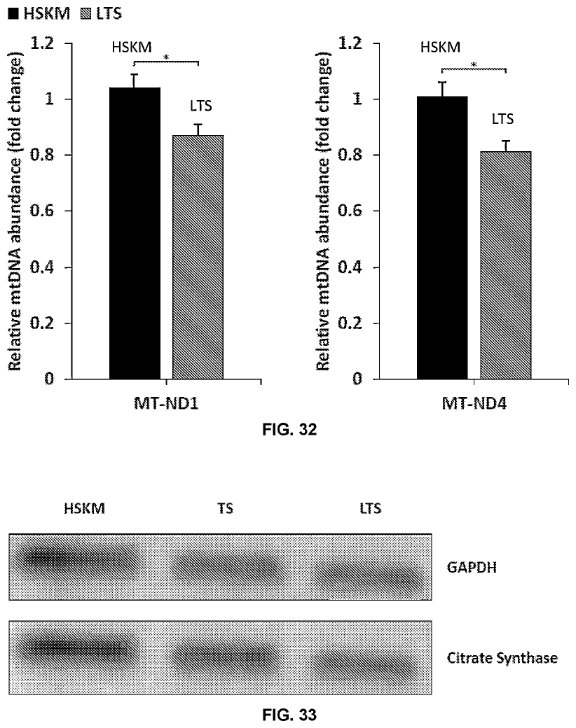

To validate that OxPhos and glycolysis were indeed upregulated, we used the Seahorse extracellular flux analyzer. Our analysis of oxygen consumption rates in the LTS progenitors revealed that LTS progenitors do indeed show significantly higher basal and maximal OxPhos rates (FIGS. 31A and 31B). Moreover, LTS progenitors also showed higher glycolysis rates (FIG. 31C). We also found that the mitochondrial membrane potential Δψₘ in LTS progenitors was significantly increased (FIG. 31D). In contrast, mitochondrial DNA and protein biogenesis did not increase, indicating that while the mitochondrial OxPhos activity levels were increased, total mitochondrial biogenesis did not increase (FIGS. 32 and 33). Since LIN28A has already been shown to bind OxPhos mRNAs to regulate OxPhos protein expression (Huang, 2012; Shyh-Chang et al., 2013a; Zhang et al., 2016), it is plausible that LIN28A-induced OxPhos would increase mtROS via an increased ETC flux and increased Δψₘ. Previous studies had shown that a high mitochondrial membrane potential Δψₘ can shift the mitochondrial ROS-producing sites to a more reduced state, thereby increasing the propensity for mitochondrial ROS (mtROS) production (Miwa and Brand, 2003; Sena and Chandel, 2012). To validate this hypothesis, we used the mitochondrial Grx1-roGFP2 reporter (Gutscher et al., 2008) to determine mtROS levels in live cells. Our results showed that LTS progenitors had mildly but significantly higher mtROS than HSKM progenitors (FIG. 31E). When we performed LC-MS/MS metabolomics profiling, we confirmed that several glycolytic intermediates and mitochondrial Krebs Cycle intermediates were indeed increased in LTS progenitors (FIGS. 31F and 31G). LC-MS/MS metabolomics also showed that the GSSG/GSH ratio, an indicator of oxidative stress, was higher in LTS progenitors (FIG. 31H).

Since mtROS is an activator of HIF1A, which in turn transactivates glycolysis genes, mtROS could also explain why the hypoxia and glycolysis signatures were upregulated (Chandel et al., 1998, 2000; Emerling et al., 2005; Majmundar et al., 2010; Semenza et al., 1994; Sena and Chandel, 2012). If LIN28A was indeed specifically optimizing mtROS to prevent senescence, then ROS-modulating drugs should have a disproportionately bigger effect on LTS progenitors than TS or HSKM progenitors. To test this hypothesis, we subjected the various muscle progenitors to a variety of ROS-modulating drugs and examined their effects on mtROS, gene expression, and proliferation rates. We found that H₂O₂ most reliably increased mtROS, whereas DTT most reliably reduced mtROS in all progenitor types (FIG. 31I). When we subjected the progenitors to H₂O₂ and DTT treatment, we found that both H₂O₂ and DTT treatments could suppress HIF1A targets (FIG. 31J) and glycolysis genes (FIG. 31K) in LTS progenitors, but not in TS progenitors, indicating that an optimal mtROS level was driving downstream HIF1A targets and glycolysis gene expression in LTS progenitors. In contrast, both H₂O₂ and DTT treatments failed to perturb the higher OxPhos mRNA levels in LTS progenitors, indicating that the OxPhos mRNAs were upstream of mtROS levels (FIG. 31L). This also supports a model whereby LIN28A→OxPhos→optimal mtROS→HIF1A-glycolysis→proliferative capacity.

LIN28A Optimizes mtROS to Activate HIF1A for Progenitor Self-Renewal

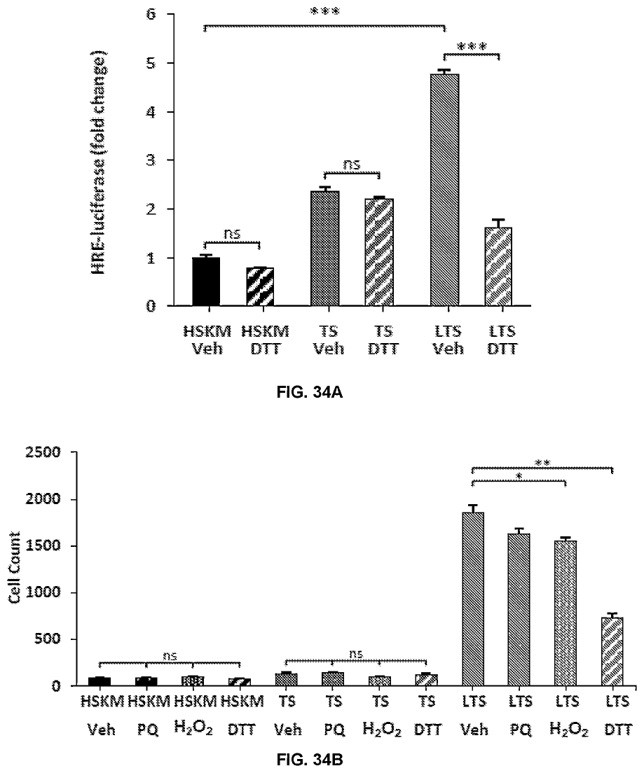

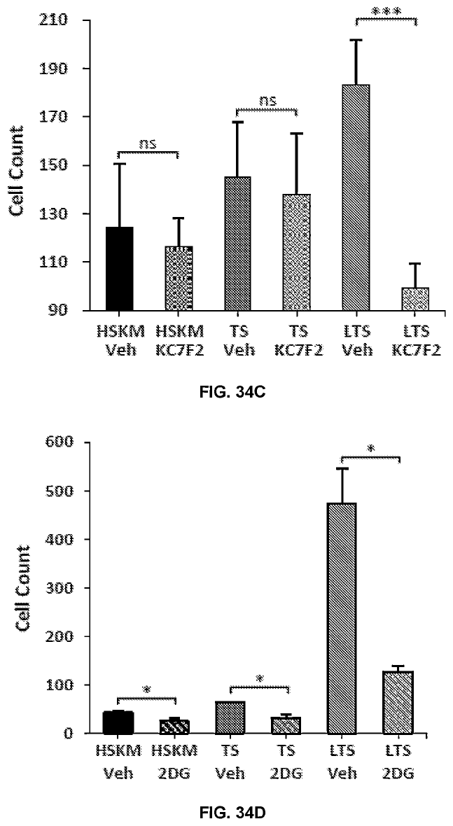

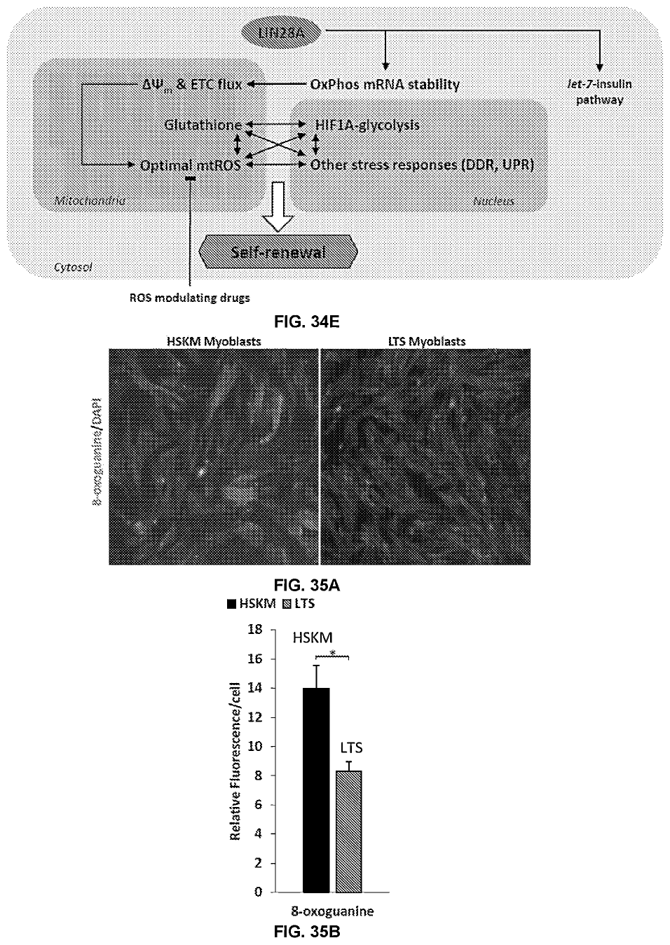

Using a HIF1A-response-element luciferase reporter (Emerling et al., 2008), we validated that LIN28A induced a HIF1A-mediated hypoxic response in LTS progenitors relative to TS and HSKM progenitors, and that this stress response was dependent on ROS levels (FIG. 34A). We also observed lower levels of the DNA oxidative damage biomarker 8-oxoguanine in LTS progenitors (FIGS. 35A-35B), as an effect of these stress responses.

Strikingly, when we examined their proliferative capacity, we found that H₂O₂ and DTT titrations both led to significant suppression of LTS progenitor proliferation in a dose-dependent fashion, with no significant effects on TS or HSKM progenitor proliferation (FIG. 34B). Consistent with the model above, we found that treatment with the HIF1A inhibitor KC7F2 significantly suppressed LTS progenitor proliferation, with no significant effects on TS or HSKM progenitor proliferation as well (FIG. 34C). In contrast, the glycolysis inhibitor 2-deoxyglucose led to mild but significant decreases in LTS, TS and HSKM progenitor proliferation (FIG. 34D), suggesting that the glycolysis-dependency is not as specific to LTS progenitors. Taken together, these results demonstrate that LIN28A specifically optimized mtROS production to promote the proliferative capacity of progenitors. Strikingly, the mtROS induced a hypoxic stress response, glycolytic response, and the DDR and UPR to rejuvenate muscle progenitors (FIG. 34E). In other contexts, such rejuvenative metabolic stress responses are collectively termed as mitohormesis (Yun and Finkel, 2014).

### DISCUSSION

We have shown that pre-senescent but old human muscle progenitors accumulate let-7 microRNAs, p53, p21^{WAF1}, p16^{INK4a}, and show telomere loss during ageing. A mini-screen with embryonic factors revealed that a minimal set of LIN28A, telomerase and shRNA knockdown of p53 (LTS) was sufficient to significantly extend the lifespan of adult human muscle progenitors. We demonstrate that these fetal-like muscle progenitors are endowed with extended self-renewal and myogenic differentiation capacities, without transformation into cancer cells. When aged and dysfunctional muscle progenitors from cancer cachexia patients were treated with LTS factors, their intrinsic self-renewal and myogenic differentiation capacities were restored, and they could now contribute to the regeneration of muscles in vivo. Mechanistically, we found that let-7 microRNAs alone and their effects on IMPS, HMGA2 and associated IGF-PI3K-mTOR signaling (Li et al., 2012; Zhu et al., 2011) could not fully explain LIN28A's effects on muscle progenitors. LIN28A prevented the senescence of myoblasts by enhancing mitochondrial OxPhos, thereby optimizing mitochondrial ROS and inducing a series of metabolic responses that promoted progenitor self-renewal—a process known as mitohormesis in other contexts. Thus, reactivation of an embryonic metabolic program with LIN28A, hTERT, and p53 inhibition could prevent the senescence phenotype of aged progenitors, without reprogramming their lineage specification, and restored their original functionalities both in vitro and in vivo.

Few studies have investigated the role of LIN28A in human progenitors. Our and others' studies have shown that mice with transgenic Lin28a show enhanced fetal-like morphogenesis and regeneration in multiple tissue compartments (Shyh-Chang et al., 2013a; Urbach et al., 2014; Yang et al., 2015; Takashima et al., 2016; Elsaeidi et al., 2018). But few studies have investigated its role in human progenitors, particularly in the skeletal muscles. Moreover, while previous studies have suggested Lin28a's role in regulating both glycolysis and OxPhos (Ma et al., 2014; Robinton et al., 2019; Shinoda et al., 2013; Zhang et al., 2016; Zhu et al., 2011), none have uncovered its importance in optimizing mtROS for mitohormesis to promote progenitor self-renewal.

Mitohormesis is defined as the coordinated compensatory response to mild mitochondrial stresses that rapidly activate nucleocytoplasmic signaling pathways, and which ultimately alter gene expression to protect the cell against stressful perturbations (Yun and Finkel, 2014). While the TS combo had little effect on mtROS and ultimately failed to enhance self-renewal, the LTS combo upregulated mtROS to an optimal level and successfully prevented adult skeletal muscle progenitor senescence. These results support the emerging notion that, while excessive mtROS can cause a variety of pro-ageing phenotypes in many contexts, mild mtROS can be beneficial at the right amounts in the right cell-types (Sena and Chandel, 2012). Interestingly recent studies have also shown that mouse embryonic myogenesis require low but not excessive amounts of mtROS, tightly regulated by Pitx2 and Pitx3 (L'honoré et al., 2014, 2018). Thus, our study validated the notion that mtROS and progenitor lifespan share a non-linear relationship, and provided a genetic combo that optimizes mtROS to extend primary human progenitors' self-renewal.

Our gene expression analyses showed that LIN28A, in the context of LTS, can robustly stimulate the HIF1A-mediated hypoxic stress response, glycolysis response, XBP1S-mediated UPR, and p53-mediated DDR. It is interesting but not surprising that, in the context of p53 knockdown, LIN28A-mediated mtROS would restore some p53 activity (Sena and Chandel, 2012). Our previous studies had shown that the p53 network is tightly-regulated and fine-tuned in progenitor cells (Le et al., 2011). While p53 protein levels still remained low overall compared to primary progenitors (FIG. 23), the restoration of some p53 activity might be critical for ensuring proper muscle progenitor self-renewal and differentiation, while preventing chromosomal instability and malignancy at the same time. Indeed, previous studies had shown that p53 is important in regulating myogenesis and many other lineages' proper differentiation (Coletti et al., 2002; Flamini et al., 2018; Schwarzkopf et al., 2006). It is also possible that the p53-mediated DDR is primarily responsible for the lower levels of 8-oxoguanine, despite higher levels of mtROS. Besides the p53-mediated DDR, the XBP1S-mediated UPR proteotoxic stress response was also activated by LIN28A-mediated mtROS. ROS-mediated protein oxidation have long been known to cause proteotoxicity, and thus activate the UPR proteotoxic stress response (Malhotra and Kaufman, 2007). In fact, recent studies have shown that the UPR is needed for proper functioning of DNA repair enzymes, including the base excision repair (BER) pathway, to remove 8-oxoguanine (Poletto et al., 2017; Xipell et al., 2016). Thus, the UPR also cross-talks with the DDR to pre-emptively protect progenitors against genotoxic stress. Another pathway stimulated by UPR is autophagy (Ron and Walter, 2007), which was previously shown to be important in rescuing geriatric mouse muscle stem cells from senescence (Garcia-Prat et al., 2016). Yet another mtROS-induced pathway that cross-talks with the p53-mediated DDR is the HIF1A-mediated hypoxic stress response (Majmundar et al., 2010). Previous studies had shown that mtROS activates HIF1A to drive the hypoxic stress response (Majmundar et al., 2010; Sena and Chandel, 2012). Moreover, it is well-known that HIF1A can transactivate glycolysis genes (Majmundar et al., 2010). Together with previous studies showing that the hypoxic stress response is important for mammalian heart regeneration and naked mole rats' resilience (Nakada et al., 2017; Park et al., 2017), our study suggests that the hypoxic stress response could be an important arm of mitohormesis in mammalian rejuvenation.

ROS can also directly stimulate glycolytic flux (Mullarky and Cantley, 2015). Glycolysis intermediates such as fructose-6-phosphate can be diverted into the PPP to fuel NADPH synthesis for reducing oxidative stress, and nucleotide synthesis for stem cell proliferation. Other glycolytic intermediates like 3-phosphoglycerate and pyruvate can be diverted into amino acid synthesis, while dihydroxyacetone-phosphate and acetyl-CoA can be diverted into lipid synthesis for anabolic growth (Shyh-Chang et al., 2013b). NADPH and amino acid synthesis are particularly important for synthesizing glutathione in the context of an oxidative stress response, which is also activated by the UPR (Ron and Walter, 2007). This is attested by the rise in the total pool of oxidized and reduced glutathione in LTS progenitors, compared to HSKM progenitors (FIG. 31H). Both NAD+ and NADH were also upregulated by LTS (FIG. 31H), but there were no significant changes in the NAD+/NADH ratio, suggesting that although changes in NAD+ and Sirt1 activity are both crucial for regulating muscle stem cell self-renewal and muscle regeneration (Ryall et al., 2015; Zhang et al., 2016), NAD+ and Sirt1 are less likely to be responsible for LTS' effects in this context. Nevertheless, the mutually reinforcing nature of these protective metabolic pathways suggests that mitohormesis is a conserved and well-coordinated program of stress responsive pathways to control cellular lifespan. LIN28A likely activates the mitohormesis program to protect progenitor cells and ensure their self-renewal during embryonic development. These mitohormetic stress response mechanisms might also shed more light on why LIN28A alone is insufficient to induce oncogenic transformation or induced pluripotent stem cell reprogramming, but facilitates cell proliferation and self-renewal.

Interestingly, LIN28A-mediated rejuvenation also induced some level of dedifferentiation to produce PAX3+ skeletal muscle progenitors (FIG. 30B). Genetic loss-of-function studies have demonstrated that Pax3+ progenitors are essential to generate all the myogenic cells in the limb, including all embryonic, fetal, and adult myoblasts and myofibers (Buckingham and Relaix, 2015; Engleka et al., 2005; Hutcheson et al., 2009; Schienda et al., 2006). Some adult satellite cells have also been reported to express Pax3 (Buckingham and Relaix, 2015; Conboy and Rando, 2002). Thus, in cooperation with hTERT and sh-p53, LIN28A could dedifferentiate PAX3− muscle progenitors into PAX3+ muscle progenitors with an increased ability to engraft and regenerate in injured skeletal muscles.

In summary, our studies provide a window on how aged progenitors can be metabolically prevented from entering senescence by inducing embryonic programs that optimize mitohormesis. Future work could focus on therapies that transiently reactivate such embryonic metabolic programs in tissue progenitors, instead of permanent transgenesis, given that senescence does play important roles in wound healing and tumor suppression (Demaria et al., 2014). Such therapies could complement current efforts to employ senolytic therapies to restore our innate regenerative capacities (Baker et al., 2011; Chang et al., 2016; Farr et al., 2017; Jeon et al., 2017; Xu et al., 2018), with implications for regenerative medicine in ageing-related diseases such as cachexia and sarcopenia.

### REFERENCES

Anchelin, M., et al. (2011). PLoS One 6, e16955.
Baker, D. J., et al. (2011). Nature 479, 232-236.
Balzer, E., et al. (2010). Development 137, 891-900.
Bar-Nur, O., et al. (2018). Stem Cell Reports 10(5), 1501-1521.
Beauséjour, C. M., et al. (2003). EMBO J. 22, 4212-4222.
Bernet, J. D., et al. (2014). Nat. Med. 20, 265-271.
Bigot, A., et al. (2008). Biol. Cell 100, 189-199.
Blau, H. M., et al. (1983). Proc. Natl. Acad. Sci. U.S.A 80, 4856-4860.
Blau, H. M., Cosgrove, B. D., and Ho, A. T. V (2015). Nat. Med. 21, 854-862.
Borchin, B., Chen, J., and Barberi, T. (2013) Stem Cell Rep. 1(6), 620-631.
Brack, A. S., and Rando, T. A. (2012). Cell Stem Cell 10, 504-514.
Briske-Anderson, et al. (1997). Proc. Soc. Exp. Biol. Med. 214, 248-257.
Buckingham, M., and Relaix, F. (2015). Cell Dev. Biol. 44, 115-125.
Capel, A. J., et al. (2019). Front. Bioeng. Biotechnol., 7, 20.
De Cecco, et al. (2019). Nature 566, 73-78.
Chandel, N. S., et al. (1998). Proc. Natl. Acad. Sci. U.S.A 95, 11715-11720.
Chandel, N. S., et al. (2000). J. Biol. Chem. 275, 25130-25138.
Chang, J., et al. (2016). Nat. Med. 22, 78-83.
Cheung, T. H., and Rando, T. A. (2013). Nat. Rev. Mol. Cell Biol. 14, 329-340.
Choi, Y., et al. (2019). Biomaterials 206: 160-169.
Christy, B., et al. (2015). p53 and rapamycin are additive. Oncotarget 6.
Chua, M.-W. J., et al. (2019). Assessment of different strategies for scalable production and proliferation of human myoblasts. Cell Prolif.doi: 10.1111/cpr.12602.
Chung, C. Y., et al. (1999). Biochem. Biophys. Res. Commun. 265, 246-251.
Coletti, D., et al. (2002). EMBO J. 21, 631-642.
Conboy, I. M., and Rando, T. A. (2002). Dev. Cell 3, 397-409.
Conboy, M. J., et al., (2010). Protocols for Adult Stem Cells, Methods in Molecular Biology™ (Methods and Protocols), vol 621. Humana Press.
Copley, M. R., et al. (2013). Nat. Cell Biol. 15, 916-925.
Cosgrove, B. D., et al. (2014). Nat. Med. 20, 255-264.
Darabi, R., et al. (2008) Nat. Med. 14, 134-143.
Darwin, C., Darwin, F., and Darwin, F. (1887). The life and letters of Charles Darwin, including an autobiographical chapter. Edited by his son, Francis Darwin. (London: John Murray).
Decary, S., Mouly, V., and Butler-Browne, G. S. (1996). Hum. Gene Ther. 7, 1347-1350.
Decary, S., et al. (1997). Hum. Gene Ther. 8, 1429-1438.
Demaria, M., et al. (2014). Dev. Cell 31, 722-733.
Deuchar, E. (1976). J. Embryol. Exp. Morphol. 35, 345-354.
Drummond, M. J., et al. (2011). Physiol. Genomics 43, 595-603.
Elsaeidi, F., et al. (2018). J. Neurosci. 38, 2246-2261.
Emerling, B. M., et al. (2005). Mol. Cell. Biol. 25, 4853-4862.
Emerling, B. M., et al. (2008). Proc. Natl. Acad. Sci. 105, 2622-2627.
Engleka, K. A., et al. (2005). Dev. Biol. 280, 396-406.
Esquenet, M., et al. (1997). J. Steroid Biochem. Mol. Biol. 62, 391-399.
Farr, J. N., et al. (2017). Nat. Med. 23, 1072-1079.
Flamini, V., et al. (2018). Stem Cell Reports 10, 970-983.
Fukawa, T., et al. (2016). Nat. Med. 22, 666-671.
Garcia-Prat, L., et al. (2016). Autophagy 12, 612-613.
Groskreutz, D. J., et al. (1994). J. Biol. Chem. 269(8), 6241-5.
Grounds, M. D. (2014). Bioarchitecture 4, 81-87.
Gussoni, E., Blau, H. M., and Kunkel, L. M. (1997). Nat. Med. 3, 970-977.
Gutscher, M., et al. (2008). Nat. Methods 5, 553-559.
Hanson, G. T., et al. (2004). J. Biol. Chem. 279, 13044-13053.
Hinds, S. et al. (2011). Biomaterials, 32(14), 3575-3583.
Huang, Y. (2012). Wiley Interdiscip. Rev. RNA 3, 483-494.
Hutcheson, D. A., et al. (2009). Genes Dev. 23, 997-1013.
Jeon, O. H., et al. (2017). Nat. Med. 23, 775-781.
Kastenhuber, E. R., and Lowe, S. W. (2017). Cell 170, 1062-1078.
Khodabukus, A. and Baar, K. (2009). Tissue Engineering Part C: Methods 15(3).
Kudlow, B. A., et al. (2008). Mol. Biol. Cell 19, 5238-5248.
L'honoré, A., et al. (2014). Dev. Cell 29, 392-405.
L'honoré, A., et al. (2018). Elife 7.
Lau, A., et al. (2019). J. Clin. Invest. 129, 4-11.
Le, M. T. N., et al. (2011). PLoS Genet. 7, e1002242.
Lee, S. J., et al. (2012). Proc. Natl. Acad. Sci. 109, E2353-E2360.
Li T. et al. (2018). Nano Research 11(10), 5240-5257.
Li, Z., et al. (2012). Dev. Cell 23, 1176-1188.
Liu, N., et al. (2017). Nat. Cell Biol. 19, 202-213.
Ma, X., et al. (2014). Nat. Commun. 5, 5212.
Majmundar, A. J., et al. (2010). Mol. Cell 40, 294-309.
Malhotra, J. D., and Kaufman, R. J. (2007). Antioxid. Redox Signal. 9, 2277-2294.
Mendell, J. R. (1995). Ann. Neurol. 37, 3-4.
Miwa, S., and Brand, M. D. (2003). Biochem. Soc. Trans. 31, 1300-1301.
Mullarky, E., and Cantley, L. C. (2015). Diverting Glycolysis to Combat Oxidative Stress.
Nakada, Y., et al. (2017). Nature 541, 222-227.
O'Driscoll, L., et al. (2006). J. Endocrinol. 191, 665-676.
Pajcini, K. V, et al. (2010). Stem Cell 7, 198-213.
Park, T. J., et al. (2017). Science. 356, 307-311.
Pearson, R. D. (1984). Acta Biotheor. 33, 51-59.
Poletto, M., et al. (2017). Nucleic Acids Res. 45, 10042-10055.
Porrello, E. R., et al. (2011). Science. 331, 1078-1080.
Price, F. D., v et al. (2014). Nat. Med. 20, 1174-1181.
Pronsato, L., et al. (2013a). Biocell 37, 1-9.
Pronsato, L., Boland, R., and Milanesi, L. (2013b). Arch. Biochem. Biophys. 530, 13-22.
Quintana, E., et al. (2008). Nature 456, 593-598.
Robinton, D. A., et al. (2019). Dev. Cell 48, 396-405.e3.
Rodier, F., Campisi, J., and Bhaumik, D. (2007). Nucleic Acids Res. 35, 7475-7484.
Ron, D., and Walter, P. (2007). Nat. Rev. Mol. Cell Biol. 8, 519-529.
Rowe, R. G., et al. (2016). J. Exp. Med. 213, 1497-1512.
Ryall, J. G., et al. (2015). Cell Stem Cell 16, 171-183.
Sadek, H. A., et al. (2014). Stem Cell Reports 3, 1.
Schienda, J., et al. (2006). Proc. Natl. Acad. Sci. 103, 945-950.
Schwarzkopf, M., et al. (2006). Genes Dev. 20, 3440-3452.
Semenza, G. L., et al. (1994). J. Biol. Chem. 269, 23757-23763.
Sena, L. A., and Chandel, N. S. (2012). Mol. Cell 48, 158-167.
Shah, M. V., Namigai, E. K. O., and Suzuki, Y. (2011). Mech. Dev. 128, 342-358.
Sharples, A. P., et al. (2011). J. Cell. Biochem. 112, 3773-3785.
Shelton, M., et al. (2016). Methods 101, 73-84.
Shinoda, G., et al. (2013). Stem Cells 31, 1563-1573.
Shyh-Chang, N., and Daley, G. Q. (2013). Cell Stem Cell 12, 395-406.
Shyh-Chang, N., et al. (2013a). Cell 155, 778-792.
Shyh-Chang, N., Daley, G. Q., and Cantley, L. C. (2013b). Development 140, 2535-2547.
Skuk, D., et al. (2010). Mol. Ther. 18, 1689-1697.
Smith-Bolton, R. K., et al. (2009). Dev. Cell 16, 797-809.
Sousa-Victor, P., et al. (2014). Nature 506, 316.
Stockley, T. L., et al. (2000). J. Lab. Clin. Med. 135(6): 484-92.
Takashima, Y., et al. (2016). Hepatology 64, 245-260.
Testa, S., et al. (2018). Materialstoday Communications 15: 120-123.
Tierney, M. T., et al. (2014). Nat. Med. 20, 1182-1186.
Tierney, M. T., et al. (2016). Cell Rep. 14, 1940-1952.
Urbach, A., et al. (2014). Genes Dev. 28, 971-982.
Van Raamsdonk, J. M., et al. (2002). J. Lab. Clin. Med. 139(1), 35-42.
Xipell, E., et al. (2016). Neuro. Oncol. 18, 1109-1119.
Xu, M., et al. (2018). Nat. Med. 24, 1246-1256.
Yang, M., et al. (2015). Development 142, 1616-1627.
Yao, K., et al. (2016). Cell Rep. 17, 165-178.
Yermalovich, A. V., et al. (2019). Nat. Commun. 10, 168.
Yin, H., Price, F., and Rudnicki, M. A. (2013). Physiol. Rev. 93, 23-67.
Yuan, J., et al. (2012). Science. 335, 1195-1200.
Yun, J., and Finkel, T. (2014). Cell Metab. 19, 757-766.
Zhang, J., et al. (2016). Cell Stem Cell 19, 66-80.
Zhu, H., et al. (2011). Cell 147, 81-94.

1. A method of delivering an agent to an individual in need thereof, comprising locally administering to the individual a composition comprising engineered myogenic wherein the engineered myogenic cells comprise muscle stem cells, myoblasts and/or differentiated cells thereof, wherein the engineered myogenic cells engraft at the site of administration in the individual, and wherein the engineered myogenic cells are genetically modified to allow delivery of the agent to the individual.

2. The method of claim 1, wherein the composition is administered intramuscularly or subcutaneously.

3. The method of claim 1, wherein the engineered myogenic cells comprise muscle stem cells, wherein the muscle stem cells are Pax7⁺ Pax3⁺ MyoD⁻ myogenin⁻, Pax7⁺ Pax3⁻ MyoD⁻ myogenin⁻, and/or Pax7⁻ Pax3⁺ MyoD myogenin⁻ cells.

4. The method of claim 1, wherein the engineered myogenic cells comprise myoblasts, wherein the myoblasts are Pax7⁻ Pax3⁺ MyoD⁺ myogenin− and/or Pax7⁺ Pax3⁻ MyoD⁺ myogenin− cells.

5. The method of claim 1, wherein the engineered myogenic cells comprise myocytes produced from muscle stem cells or myoblasts; wherein the myocytes are Pax3⁻ Pax7⁻ MyoD⁺ myogenin⁺ cells.

6. The method of claim 1, wherein the engineered myogenic cells are reprogrammed myogenic cells, wherein:
(i) the engineered myogenic cells are rejuvenated and/or de-differentiated myogenic cells produced from adult myogenic cells; or
(ii) the engineered myogenic cells are transdifferentiated myogenic cells produced from adult somatic cells.

7. The method of claim 1, wherein the composition is a suspension of engineered myogenic cells, wherein the composition is administered by injection.

8. The method of claim 1, wherein the composition is a muscle construct comprising engineered myogenic cells, wherein the composition is administered as a local implant.

9. The method of claim 8, wherein:
(i) the muscle construct comprise myotubes;
(ii) the muscle construct comprise myofibers; and/or
(iii) the muscle construct comprises PAX7⁺ myogenic cells.

10. The method of claim 1, wherein the composition further comprises a carrier.

11. The method of claim 1, wherein the engineered myogenic cells are not produced from an immortalized cell line.

12. The method of claim 1, wherein the engrafted engineered myogenic cells form a muscle tissue in the individual, wherein the muscle tissue allows delivery of the agent to the individual.

13. The method of claim 12, wherein the muscle tissue produces the agent.

14. The method of claim 12, wherein the muscle tissue allows local delivery of the agent to the individual.

15. The method of claim 12, wherein the muscle tissue allows systemic delivery of the agent to the individual.

16. The method of claim 12, wherein the muscle tissue recruits blood vessels from surrounding tissues in the individual, and/or wherein the muscle tissue is fused to surrounding tissues in the individual.

17. (canceled)

18. The method of claim 12, wherein the muscle tissue is removed from the individual after delivery of the agent to the individual for at least about 2 months.

19. The method of claim 1, wherein:
(i) the composition is administered once to the individual; and/or
(ii) the agent is delivered to the individual for at least about 2 months upon administration of the engineered myogenic cells.

20. The method of claim 1, wherein the engineered myogenic cells comprise a heterologous nucleic acid sequence encoding the agent.

21. The method of claim 1, wherein the agent is selected from the group consisting of a metabolite, a nutrient, a small molecule drug, a biologic, a virus, an extracellular vesicle, a vaccine and a reporter.

22. The method of claim 1, wherein:
(i) the agent is secreted by the engineered myogenic cells; or
(ii) the agent is expressed on the cell surface of the engineered myogenic cells.

23. The method of claim 1, wherein the engineered myogenic cells allow delivery of a plurality of agents.

24. The method of claim 1, wherein the composition comprises at least about 10⁵ of the engineered myogenic.

25. The method of claim 1, herein the engineered myogenic cells are autologous or allogeneic.

26. The method of claim 1, wherein the engineered myogenic cells are non-immunogenic to the individual and/or nontumorigenic.

27. (canceled)

28. The method of claim 1, wherein the individual is a human individual.

29. A method of treating or diagnosing a disease or condition in an individual, comprising delivering an agent to the individual using the method of claim 1, wherein the agent treats or diagnoses the disease or condition.

30. A method of preparing a non-human animal model of a disease or condition, comprising delivering an agent to a non-human animal using the method of claim 1, wherein the agent is associated with the disease or condition.

31. A composition comprising engineered myogenic cells, a hydrogel carrier comprising extracellular matrix molecules, and fibrin, wherein the engineered myogenic cells comprise muscle stem cells, myoblasts and/or differentiated cells thereof, and wherein the engineered myogenic cells are intermixed with the carrier.

32-33. (canceled)

34. A method of producing a reprogrammed myogenic cell from an adult myogenic cell, comprising:
(a) introducing into the adult myogenic cell an embryonic regulator, a telomere-associated regulator, and a cell cycle regulator to provide a transduced myogenic cell; and
(b) culturing the transduced myogenic cell under conditions to obtain a reprogrammed myogenic cell.

35-40. (canceled)
